(19) United States
(12) Patent Application Publication
(10) Pub. No.: US 2019/0170734 A1
(43) Pub. Date: Jun. 6, 2019

(54) Compact Illuminator, Imaging and Systems and the Use of the Same

(71) Applicant: Essenlix Corporation, Monmouth Junction, NJ (US)

(72) Inventors: Stephen Y. Chou, Princeton, NJ (US); Wei Ding, East Windsor, NJ (US); Ji Qi, Hillsborough, NJ (US)

(73) Assignee: Essenlix Corporation, Monmouth Junction, NJ (US)

(21) Appl. No.: 16/172,465

(22) Filed: Oct. 26, 2018

Related U.S. Application Data
(60) Provisional application No. 62/577,503, filed on Oct. 26, 2017.

(51) Int. Cl. G01N 33/52; G01N 33/53; G01N 15/14; G01N 21/64; B01L 3/00

(52) U.S. Cl. CPC G01N 33/52; G01N 33/5304; G01N 15/1463; G01N 2015/0065; G01N 21/6486; B01L 3/5085; B01L 3/50273; G01N 21/6428

(57) ABSTRACT

Among other things, the present invention is related to devices and methods for imaging a liquid sample between two plates.

### CROSS REFERENCING

This application claims the benefit of U.S. Provisional Application No. 62/577,503, filed on Oct. 26, 2017, the disclosure of which is incorporated herein in its entirety for all purposes.

### FIELD

Among other things, the present invention is related to devices and methods for imaging a liquid sample between two plates.

### BACKGROUND

In biological and chemical assays (e.g. diagnostic testing), a compressed open flow (COF) of a liquid sample can have many advantages over other methods in handing a flowable sample (i.e. liquid). In COF, two planar plates that are movable relative to each other are used, and a flowable sample is first deposited on one or both plates when the two plates are in an open configuration, followed by bring the two plates together to compress the sample between two plates; wherein the compression reduces a thickness of the sample and makes the sample flow into open spaces between the plates.

In order to capture a good image of the sample between the two plates, it is desirable to illuminate the sample with uniform illumination. It is desirable to generate such uniform illumination using a passive illuminator that receives light from the light source on a smartphone.

### SUMMARY

A device for illuminating and imaging an object, comprising an imager having a lens; and a passive illuminator; and an adaptor housing that has an exit aperture for positioning an imager wherein the passive illuminator is on the adaptor; and wherein the adaptor housing is configured to reduce ambient light outside the adaptor housing entering inside adaptor housing.

The device of any embodiment of the present disclosure, wherein the adaptor housing further comprises a slot for inserting a sample holder into the adaptor housing and the passive illuminator is position around and outside peripheral of the exit aperture.

An apparatus for illuminating and imaging an object, comprising a mobile phone that has a camera and a light source; and the device of claim 1.

A method for illuminating and imaging an object, the method comprising the steps of providing the device of claim 1; providing an adaptor housing; and providing a mobile phone that has a camera and a light source, wherein the adaptor housing has an exit aperture for positioning the imager, wherein the adaptor housing is configured to reduce ambient light outside the adaptor housing entering the adaptor housing, and wherein the adaptor housing is configured to attach to the mobile phone.

A method for illuminating and imaging a liquid sample between two parallel plates in an assay device, the method comprising the steps of impinging light into one or two light-guides each connecting to one end of the passive illuminator; causing the impinging light to travel through each light-guide to reach the corresponding end of the passive illuminator; causing light to be emitted from a side wall of the passive illuminator after the impinging light enters the corresponding end of the passive illuminator; generating illumination light from the light emitted from the side wall of the passive illuminator; illuminating the liquid sample through one of the parallel plates with the illumination light; and imaging the liquid sample with an imaging sensor through a lens.

An apparatus for using with a smartphone to read an assay device having two parallel plates, the smartphone having a camera and a light source, the apparatus comprising one or two light-guides each having an end thereof aligned with the entrance aperture of the optics chamber to cause light entering such end of the light-guide to travel through the light-guide to reach a corresponding end of the passive illuminator; and a passive illuminator for illuminating a liquid sample between the two parallel plates in the assay device by generating diffusive light sideways from areas surrounding an optical axis of a lens in the camera of the smartphone, wherein the passive illuminator has a first end optically coupled to a second end of the light-guide to cause light received at the first end of each light-guide to travel through the light-guide to enter the first end of the passive illuminator.

The apparatus of any embodiment of the present disclosure, further comprising a diffuser for generating diffusive light sideways from areas surrounding the optical axis of the lens in the camera of the smartphone to illuminate the liquid sample between the two parallel plates in the assay device.

The device, apparatus, or method of any embodiment of the present disclosure, wherein the passive illuminator is in the form of a ring configured to surround an optical axis of a lens in the camera of the smartphone when the apparatus is engaged with the smartphone.

The device, apparatus, or method of any embodiment of the present disclosure, further comprising an auxiliary lens having an optical axis thereof aligned with the optical axis of the lens in the camera of the smartphone when the apparatus is engaged with the smartphone, wherein the auxiliary lens has a diameter that is at least 2 mm, 3 mm, 4 mm, 5 mm, 10 mm, 15 mm, 20 mm, 25 mm, 30 mm, 40 mm, or 50 mm, or in a range between any of the two values.

The device, apparatus, or method of any embodiment of the present disclosure, further comprising an optical condenser configured to be placed in front of the light source of the smartphone when the apparatus is engaged with the smartphone, or an optical condenser aligned with the entrance aperture of the optics chamber.

The device, apparatus, or method of any embodiment of the present disclosure, wherein the diffuser comprises at least one of the following polished surfaces on both sides; a volume diffusive material which can be but not limited to opaque white glass and opaque white plastic, wherein the transmissivity of the volume diffusive material is at least 40%, 60%, 80%, 90% or in a range between any of the two values; and at least one textured surface, wherein the volume diffusive material can be but not limited to opaque white glass and opaque white plastic, wherein the transmissivity of the volume diffusive material is at least 40%, 60%, 80%, 90% or in a range between any of the two values, and wherein the grit of the textured surface is at least 100, 200, 400, 600, 800, 1,000, 2,000, or in a range between any of the two values.

The device, apparatus, or method of any embodiment of the present disclosure, further comprising a reflector configured to reflect light emitted from the passive illuminator towards the diffuser, or a reflector configured to reflect light emitted from the passive illuminator towards the exposure aperture of the optics chamber.

The device, apparatus, or method of any embodiment of the present disclosure, further comprising a receptacle slot operative to hold the assay device while exposing at least part of a first one of the two parallel plate in the assay device to a lens in the camera of the smartphone when the assay device is inserted into the receptacle slot and the apparatus is engaged with the smartphone; or a receptacle slot operative to hold the assay device while exposing at least part of a first one of the two parallel plate in the assay device to the exposure aperture of the optics chamber when the assay device is inserted into the receptacle slot; or a receptacle slot having two side walls forming a cavity for holding the assay device therein, wherein one of the two side walls has an opening for forming the exposure aperture of the optics chamber, wherein the light-guide has the first end configured to receive light from the light source of the smartphone when the apparatus is engaged with the smartphone.

The device, apparatus, or method of any embodiment of the present disclosure, further comprising an optics chamber having an entrance aperture; an exit aperture at a first side of the optics chamber; and an exposure aperture at a second side of the optics chamber, wherein the light-guide has the first end aligned with the entrance aperture of the optics chamber, wherein each of the entrance aperture, the exit aperture, and the exposure aperture is covered with a window.

The device, apparatus, or method of any embodiment of the present disclosure, wherein the exit aperture at the first side of the optics chamber is aligned with the exposure aperture at the second side of the optics chamber for exposing optically at least part of the first one of the two parallel plate in the assay device to the exit aperture of the optics chamber through the exposure aperture of the optics chamber when the assay device is inserted into the receptacle slot.

The device, apparatus, or method of any embodiment of the present disclosure, further comprising an auxiliary lens aligned with the exit aperture of the optics chamber, or an auxiliary lens located between the passive illuminator and the receptacle slot operative to hold the assay device, or an auxiliary having an optical axis thereof coaxially aligned with an optical axis of the lens in the camera of the smartphone when the apparatus is engaged with the smartphone.

The device, apparatus, or method of any embodiment of the present disclosure, further comprising a diffuser placed at a predetermined distance from the passive illuminator; and an opening on the diffuser configured to expose to the camera of the smartphone at least a part of the exposure aperture in the optics chamber when the apparatus is engaged with the smartphone, wherein the diffuser is configured to intercept all light path directly between the passive illuminator and the exposure aperture of the optics chamber.

The device, apparatus, or method of any embodiment of the present disclosure, wherein the distance between the passive illuminator and the outside peripheral of the imager is in a range of 2 mm to 50 mm.

The device, apparatus, or method of any embodiment of the present disclosure, wherein the passive illuminator is formed by a side illumination fiber, wherein the side illumination fiber comprises a core and a cladding layer, and wherein the ratio of transmissivity to reflectivity at the interface between the core and cladding layer is at least 1:100, 1:10, 1:1, or in a range between any of the two values, or wherein the passive illuminator is formed by a side illumination fiber, and wherein the side illumination fiber is made of but not limited to flexible polymers, plastic, glass and rigid dielectric materials.

The device, apparatus, or method of any embodiment of the present disclosure, wherein the passive illuminator is rotationally symmetric or rotationally non-symmetric, or the passive illuminator is in the form of a circle having a diameter thereof in a range between 5 mm and 100 mm, or the passive illuminator is in the form of a convex polygon, a star polygon, an ellipse, or a circle, or the passive illuminator is formed by a single piece of side illumination fiber or by at least two segments of side illumination fibers, or the passive illuminator has a substantially uniform cross-section.

### BRIEF DESCRIPTION OF THE DRAWINGS

The skilled artisan will understand that the drawings, described below, are for illustration purposes only. The drawings are not intended to limit the scope of the present teachings in any way. In some Figures, the drawings are in scale. In the figures that present experimental data points, the lines that connect the data points are for guiding a viewing of the data only and have no other means.

FIG. 1 shows an illustration of a CROF (Compressed Regulated Open Flow) embodiment. Panel (a) illustrates a first plate and a second plate wherein the first plate has spacers. Panel (b) illustrates depositing a sample on the first plate (shown), or the second plate (not shown), or both (not shown) at an open configuration. Panel (c) illustrates (i) using the two plates to spread the sample (the sample flow between the plates) and reduce the sample thickness, and (ii) using the spacers and the plate to regulate the sample thickness at the closed configuration. The inner surface of each plate may have one or a plurality of binding sites and or storage sites (not shown).

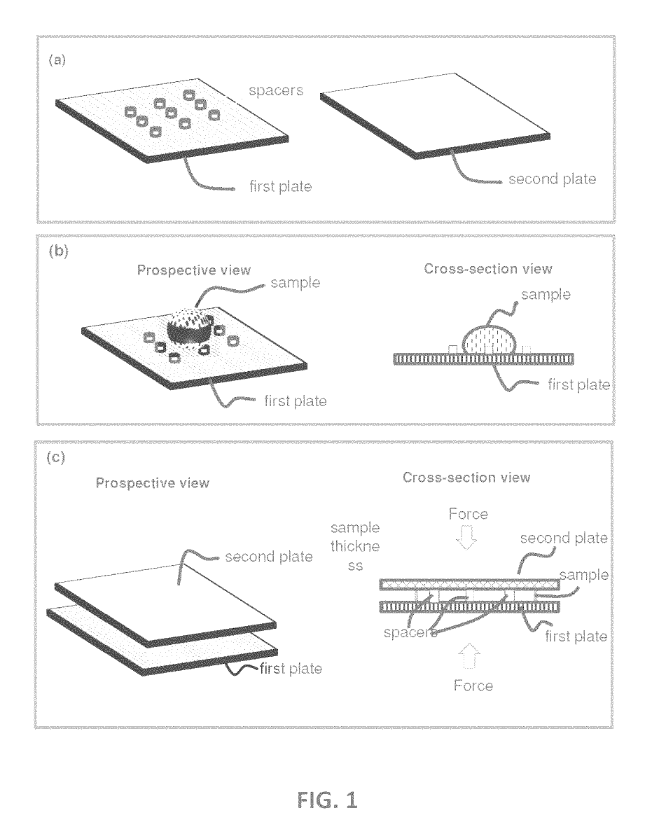

FIG. 2 shows a passive illuminator that is positioned around the outside peripheral of an imager lens in accordance with some embodiment.

FIG. 3 shows a passive illuminator and a diffuser that are positioned around the outside peripheral of an imager lens in accordance with some embodiment.

FIG. 4 shows a passive illuminator and a diffuser that are positioned around the outside peripheral of an imager lens in accordance with some embodiment.

FIG. 5A shows one implementation of a passive illuminator that are positioned around the outside peripheral of an imager lens in accordance with some embodiment.

FIG. 5B shows one implementation of a passive illuminator and a diffuser that are positioned around the outside peripheral of an imager lens in accordance with some embodiment.

FIG. 6 is a three-dimensional view of the diffusive plate in FIG. 5B.

FIG. 7 shows another implementation of a passive illuminator and a diffuser that are positioned around the outside peripheral of an imager lens in accordance with some embodiment.

FIG. 8 shows one implementation of a passive illuminator and a diffuser that are positioned around the outside peripheral of an imager lens in accordance with some embodiment.

FIG. 9 shows another implementation of a passive illuminator and a diffuser that are positioned around the outside peripheral of an imager lens in accordance with some embodiment.

FIG. 10A is a schematic of an adaptor for using with a smartphone to read an assaying device in accordance with some embodiment.

FIG. 10B is a schematic of a passive illuminator in the optical adaptor of FIG. 10A in accordance with some embodiments.

FIG. 11A shows a schematic view showing details of the system reading an assaying device in accordance with some embodiments.

FIG. 11B shows a schematic view showing details of the system reading an assaying device in accordance with some embodiments.

FIG. 11C shows a schematic view showing details of the system reading an assaying device in accordance with some embodiments.

FIGS. 12A-12B shows a passive illuminator that is positioned around the outside peripheral of an imager lens in accordance with some embodiment. FIGS. 12A-12B shows a diffuser that is configured to intercept all light path directly between the passive illuminator and the assaying device. In FIGS. 12A-12B, the upper surface of the passive illuminator can be separated from the face of the smartphone by a distance S. This distance S can be any value between 5 mm and 50 mm.

### DETAILED DESCRIPTION OF EXEMPLARY EMBODIMENTS

The following detailed description illustrates some embodiments of the invention by way of example and not by way of limitation. If any, the section headings and any subtitles used herein are for organizational purposes only and are not to be construed as limiting the subject matter described in any way. The contents under a section heading and/or subtitle are not limited to the section heading and/or subtitle, but apply to the entire description of the present invention.

The citation of any publication is for its disclosure prior to the filing date and should not be construed as an admission that the present claims are not entitled to antedate such publication by virtue of prior invention. Further, the dates of publication provided can be different from the actual publication dates which can need to be independently confirmed.

It should be noted that the Figures do not intend to show the elements in strict proportion. For clarity purposes, some elements are enlarged when illustrated in the Figures. The dimensions of the elements should be delineated from the descriptions herein provided and incorporated by reference.

### Definitions

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are now described.

The terms “labeled analyte” and “bound label” are interchangeable. The phrase “labeled analyte” refers to an analyte that is detectably labeled with a light emitting label such that the analyte can be detected by assessing the presence of the label. A labeled analyte may be labeled directly (i.e., the analyte itself may be directly conjugated to a label, e.g., via a strong bond, e.g., a covalent or non-covalent bond), or a labeled analyte may be labeled indirectly (i.e., the analyte is bound by a secondary capture agent that is directly labeled).

The terms “unbound label” and “background” are interchangeable, with understanding that the signal of “unbound label” includes signals from other background that are not “unbound label”.

The term “lateral area” refers to the area that is in parallel with the plate.

The term “analyte-concentration area” refers to an area of a surface where the area has a higher affinity to bind the labeled analyte/bound label (or to bind an analyte what later binds a label) than the rest area of the surface.

The term “lateral distance between two neighboring analyte concentration areas” or “IACD (inter analyte concentration-area distance)” refers to the distance between the average center of each analyte concentration area. For example, if each of the analyte concentration area has a circular shape in lateral shape, the IACD is the distance between the centers of the two circles. Another example, if each of the two analyte concentration areas is a vertical plane, then the IACD is the lateral distance between the two planes.

The term “diffusion parameter” or “DP” as used herein refers to a parameter that is equal to \sqrt{Dt}, wherein D is the diffusion constant of the analyte in the sample and the t is the intended assay time (i.e. the diffusion parameter is equal to the square-root of the diffusion constant of the analyte in the sample multiplying the intended assay time); wherein the intended assay time is a time parameter. For example, if the diffusion constant of the analyte in the sample is 1×10⁻⁷ cm²/s, the intended assay time is 60 sec, then the diffusion parameter is 24 μm (micron). Some of the common analyte diffusion constants are IgG in PBS: 3×10⁻⁷ cm²/s, IgG in blood: 1×10⁻⁷ cm²/s, and 20 bp DNA in blood: 4×10⁻⁷ cm²/s.

The term “bead” as used herein refers to a nano-scale or micro-scale three-dimensional object, regardless of its shape and material.

The term “specifically capture” means that a capture agent selectively bound an analyte that will be detected.

The terms “specific binding” and “selective binding” refer to the ability of a capture agent to preferentially bind to a particular target molecule that is present in a heterogeneous mixture of different target molecule. A specific or selective binding interaction will discriminate between desirable (e.g., active) and undesirable (e.g., inactive) target molecules in a sample, typically more than about 10 to 100-fold or more (e.g., more than about 1000- or 10,000-fold).

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

The terms “polynucleotide”, “nucleotide”, “nucleotide sequence”, “nucleic acid”, “nucleic acid molecule”, “nucleic acid sequence” and “oligonucleotide” are used interchangeably, and can also include plurals of each respectively depending on the context in which the terms are utilized. They refer to a polymeric form of nucleotides of any length, either deoxyribonucleotides (DNA) or ribonucleotides (RNA), or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA, ribozymes, small interfering RNA, (siRNA), microRNA (miRNA), small nuclear RNA (snRNA), cDNA, recombinant polynucleotides, branched polynucleotides, plasmids, vectors, isolated DNA (A, B and Z structures) of any sequence, PNA, locked nucleic acid (LNA), TNA (treose nucleic acid), isolated RNA of any sequence, nucleic acid probes, and primers. LNA, often referred to as inaccessible RNA, is a modified RNA nucleotide. The ribose moiety of an LNA nucleotide is modified with an extra bridge connecting the 2′ and 4′ carbons. The bridge “locks” the ribose in the 3′-endo structural conformation, which is often found in the A-form of DNA or RNA, which can significantly improve thermal stability.

The term “capture agent” as used herein, refers to a binding member, e.g. nucleic acid molecule, polypeptide molecule, or any other molecule or compound, that can specifically bind to its binding partner, e.g., a second nucleic acid molecule containing nucleotide sequences complementary to a first nucleic acid molecule, an antibody that specifically recognizes an antigen, an antigen specifically recognized by an antibody, a nucleic acid aptamer that can specifically bind to a target molecule, etc. A capture agent may concentrate the target molecule from a heterogeneous mixture of different molecules by specifically binding to the target molecule. Binding may be non-covalent or covalent. The affinity between a binding member and its binding partner to which it specifically binds when they are specifically bound to each other in a binding complex is characterized by a KD (dissociation constant) of 10⁻⁵ M or less, 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, including 10⁻¹⁶ M or less. “Affinity” refers to the strength of binding, increased binding affinity being correlated with a lower KD.

The term “a secondary capture agent” which can also be referred to as a “detection agent” refers a group of biomolecules or chemical compounds that have highly specific affinity to the antigen. The secondary capture agent can be strongly linked to an optical detectable label, e.g., enzyme, fluorescence label, or can itself be detected by another detection agent that is linked to an optical detectable label through bioconjugation (Hermanson, “Bioconjugate Techniques” Academic Press, 2nd Ed., 2008).

The term “capture agent-reactive group” refers to a moiety of chemical function in a molecule that is reactive with capture agents, i.e., can react with a moiety (e.g., a hydroxyl, sulfhydryl, carboxyl or amine group) in a capture agent to produce a stable strong, e.g., covalent bond.

The term “antibody,” as used herein, is meant a protein consisting of one or more polypeptides substantially encoded by all or part of the recognized immunoglobulin genes. The recognized immunoglobulin genes, for example in humans, include the kappa (κ), lambda (λ), and heavy chain genetic loci, which together comprise the myriad variable region genes, and the constant region genes mu (μ), delta (δ), gamma (γ), sigma (σ), and alpha (α) which encode the IgM, IgD, IgG, IgE, and IgA antibody “isotypes” or “classes” respectively. Antibody herein is meant to include full length antibodies and antibody fragments, and may refer to a natural antibody from any organism, an engineered antibody, or an antibody generated recombinantly for experimental, therapeutic, or other purposes. The term “antibody” includes full length antibodies, and antibody fragments, as are known in the art, such as Fab, Fab′, F(ab′)2, Fv, scFv, or other antigen-binding subsequences of antibodies, either produced by the modification of whole antibodies or those synthesized de novo using recombinant DNA technologies.

The terms “antibody epitope,” “epitope,” “antigen” are used interchangeably herein to refer to a biomolecule that is bound by an antibody. Antibody epitopes can include proteins, carbohydrates, nucleic acids, hormones, receptors, tumor markers, and the like, and mixtures thereof. An antibody epitope can also be a group of antibody epitopes, such as a particular fraction of proteins eluted from a size exclusion chromatography column. Still further, an antibody epitope can also be identified as a designated clone from an expression library or a random epitope library.

An “allergen,” as used herein is a substance that elicits an allergic, inflammatory reaction in an individual when the individual is exposed to the substance, e.g., by skin contact, ingestion, inhalation, eye contact, etc. An allergen may include a group of substances that together elicit the allergic reaction. Allergens may be found in sources classified by the following groups: natural and artificial fibers (cotton, linen, wool, silk, teak, etc., wood, straw, and other dust); tree pollens (alder, birch, hazel, oak, poplar, palm, and others); weeds and flowers (ambrosia, artemisia, and others); grasses and corns (fescue, timothy grass, rye, wheat, corn, bluegrass, and others); drugs (antibiotics, antimicrobial drugs, analgetics and non-steroid anti-inflammatory drugs, anesthetics and muscle relaxants, hormones, and others); epidermal and animal allergens (epithelium of animals, feathers of birds, sera, and others); molds and yeasts (Penicillium notation, Cladosporium spp., Aspergillus fumigatus, Mucor racemosus, and others); insect venoms; preservatives (butylparaben, sorbic acid, benzoate, and others); semen (ejaculate); parasitic and mite allergens (ascarids, Dermatophagoides pteronyssinus, Dermatophagoides farinae, Euroglyphus maynei, and others); occupational and hobby allergens (coffee beans, formaldehyde, latex, chloramine, dyes, and others); food allergens (egg products, dairy products and cheeses, meat products, fish and seafood, soy products, mushrooms, flours and cereals, vegetables, melons and gourds, beans, herbs and spices, nuts, citrus and other fruits, berries, teas and herbs, nutritional supplements, and other products), etc.

The term “Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these.

As is known to one skilled in the art, hybridization can be performed under conditions of various stringency. Suitable hybridization conditions are such that the recognition interaction between a capture sequence and a target nucleic acid is both sufficiently specific and sufficiently stable. Conditions that increase the stringency of a hybridization reaction are widely known and published in the art. See, for example, Green, et al., (2012), infra.

The term “protein” refers to a polymeric form of amino acids of any length, i.e. greater than 2 amino acids, greater than about 5 amino acids, greater than about 10 amino acids, greater than about 20 amino acids, greater than about 50 amino acids, greater than about 100 amino acids, greater than about 200 amino acids, greater than about 500 amino acids, greater than about 1000 amino acids, greater than about 2000 amino acids, usually not greater than about 10,000 amino acids, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like. Also included by these terms are polypeptides that are post-translationally modified in a cell, e.g., glycosylated, cleaved, secreted, prenylated, carboxylated, phosphorylated, etc., and polypeptides with secondary or tertiary structure, and polypeptides that are strongly bound, e.g., covalently or non-covalently, to other moieties, e.g., other polypeptides, atoms, cofactors, etc.

The term “complementary” as used herein refers to a nucleotide sequence that base-pairs by hydrogen bonds to a target nucleic acid of interest. In the canonical Watson-Crick base pairing, adenine (A) forms a base pair with thymine (T), as does guanine (G) with cytosine (C) in DNA. In RNA, thymine is replaced by uracil (U). As such, A is complementary to T and G is complementary to C. Typically, “complementary” refers to a nucleotide sequence that is fully complementary to a target of interest such that every nucleotide in the sequence is complementary to every nucleotide in the target nucleic acid in the corresponding positions. When a nucleotide sequence is not fully complementary (100% complementary) to a non-target sequence but still may base pair to the non-target sequence due to complementarity of certain stretches of nucleotide sequence to the non-target sequence, percent complementarily may be calculated to assess the possibility of a non-specific (off-target) binding. In general, a complementary of 50% or less does not lead to non-specific binding. In addition, a complementary of 70% or less may not lead to non-specific binding under stringent hybridization conditions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” as used herein denotes single stranded nucleotide multimers of from about 10 to 200 nucleotides and up to 300 nucleotides in length, or longer, e.g., up to 500 nucleotides in length or longer. Oligonucleotides may be synthetic and, in certain embodiments, are less than 300 nucleotides in length.

The term “attaching” as used herein refers to the strong, e.g., covalent or non-covalent, bond joining of one molecule to another.

The term “surface attached” as used herein refers to a molecule that is strongly attached to a surface.

The term “sample” as used herein relates to a material or mixture of materials containing one or more analytes or entity of interest. In particular embodiments, the sample may be obtained from a biological sample such as cells, tissues, bodily fluids, and stool. Bodily fluids of interest include but are not limited to, amniotic fluid, aqueous humour, vitreous humour, blood (e.g., whole blood, fractionated blood, plasma, serum, etc.), breast milk, cerebrospinal fluid (CSF), cerumen (earwax), chyle, chime, endolymph, perilymph, feces, gastric acid, gastric juice, lymph, mucus (including nasal drainage and phlegm), pericardial fluid, peritoneal fluid, pleural fluid, pus, rheum, saliva, sebum (skin oil), semen, sputum, sweat, synovial fluid, tears, vomit, urine and exhaled condensate. In particular embodiments, a sample may be obtained from a subject, e.g., a human, and it may be processed prior to use in the subject assay. For example, prior to analysis, the protein/nucleic acid may be extracted from a tissue sample prior to use, methods for which are known. In particular embodiments, the sample may be a clinical sample, e.g., a sample collected from a patient.

The term “analyte” refers to a molecule (e.g., a protein, peptides, DNA, RNA, nucleic acid, or other molecule), cells, tissues, viruses, and nanoparticles with different shapes.

The term “assaying” refers to testing a sample to detect the presence and/or abundance of an analyte.

As used herein, the terms “determining,” “measuring,” and “assessing,” and “assaying” are used interchangeably and include both quantitative and qualitative determinations.

As used herein, the term “light-emitting label” refers to a label that can emit light when under an external excitation. This can be luminescence. Fluorescent labels (which include dye molecules or quantum dots), and luminescent labels (e.g., electro- or chemi-luminescent labels) are types of light-emitting label. The external excitation is light (photons) for fluorescence, electrical current for electroluminescence and chemical reaction for chemi-luminescence. An external excitation can be a combination of the above.

The terms “hybridizing” and “binding”, with respect to nucleic acids, are used interchangeably.

The term “capture agent/analyte complex” is a complex that results from the specific binding of a capture agent with an analyte. A capture agent and an analyte for the capture agent will usually specifically bind to each other under “specific binding conditions” or “conditions suitable for specific binding”, where such conditions are those conditions (in terms of salt concentration, pH, detergent, protein concentration, temperature, etc.) which allow for binding to occur between capture agents and analytes to bind in solution. Such conditions, particularly with respect to antibodies and their antigens and nucleic acid hybridization are well known in the art (see, e.g., Harlow and Lane (Antibodies: A Laboratory Manual Cold Spring Harbor Laboratory, Cold Spring Harbor, N.Y. (1989) and Ausubel, et al, Short Protocols in Molecular Biology, 5th ed., Wiley & Sons, 2002).

The term “specific binding conditions” and “conditions suitable for binding,” as used herein with respect to binding of a capture agent to an analyte, e.g., a biomarker, a biomolecule, a synthetic organic compound, an inorganic compound, etc., refers to conditions that produce nucleic acid duplexes or, protein/protein (e.g., antibody/antigen) complexes, protein/compound complexes, aptamer/target complexes that contain pairs of molecules that specifically bind to one another, while, at the same time, disfavor to the formation of complexes between molecules that do not specifically bind to one another. Specific binding conditions are the summation or combination (totality) of both hybridization and wash conditions, and may include a wash and blocking steps, if necessary. For nucleic acid hybridization, specific binding conditions can be achieved by incubation at 42° C. in a solution: 50% formamide, 5×SSC (150 mM NaCl, 15 mM trisodium citrate), 50 mM sodium phosphate (pH7.6), 5×Denhardt's solution, 10% dextran sulfate, and 20 ug/ml denatured, sheared salmon sperm DNA, followed by washing the filters in 0.1×SSC at about 65° C.

For binding of an antibody to an antigen, specific binding conditions can be achieved by blocking a first plate containing antibodies in blocking solution (e.g., PBS with 3% BSA or non-fat milk), followed by incubation with a sample containing analytes in diluted blocking buffer. After this incubation, the first plate is washed in washing solution (e.g. PBS+TWEEN 20) and incubated with a secondary capture antibody (detection antibody, which recognizes a second site in the antigen). The secondary capture antibody may be conjugated with an optical detectable label, e.g., a fluorophore such as IRDye800CW, Alexa 790, Dylight 800. After another wash, the presence of the bound secondary capture antibody may be detected. One of skill in the art would be knowledgeable as to the parameters that can be modified to increase the signal detected and to reduce the background noise.

A subject may be any human or non-human animal. A subject may be a person performing the instant method, a patient, a customer in a testing center, etc.

An “analyte,” as used herein is any substance that is suitable for testing in the present invention.

As used herein, a “diagnostic sample” refers to any biological sample that is a bodily byproduct, such as bodily fluids, that has been derived from a subject. The diagnostic sample may be obtained directly from the subject in the form of liquid, or may be derived from the subject by first placing the bodily byproduct in a solution, such as a buffer. Exemplary diagnostic samples include, but are not limited to, saliva, serum, blood, sputum, urine, sweat, lacrima, semen, feces, breath, biopsies, mucus, etc.

As used herein, an “environmental sample” refers to any sample that is obtained from the environment. An environmental sample may include liquid samples from a river, lake, pond, ocean, glaciers, icebergs, rain, snow, sewage, reservoirs, tap water, drinking water, etc.; solid samples from soil, compost, sand, rocks, concrete, wood, brick, sewage, etc.; and gaseous samples from the air, underwater heat vents, industrial exhaust, vehicular exhaust, etc. Typically, samples that are not in liquid form are converted to liquid form before analyzing the sample with the present invention.

As used herein, a “foodstuff sample” refers to any sample that is suitable for animal consumption, e.g., human consumption. A foodstuff sample may include raw ingredients, cooked food, plant and animal sources of food, preprocessed food as well as partially or fully processed food, etc. Typically, samples that are not in liquid form are converted to liquid form before analyzing the sample with the present invention.

The term “diagnostic,” as used herein, refers to the use of a method or an analyte for identifying, predicting the outcome of and/or predicting treatment response of a disease or condition of interest. A diagnosis may include predicting the likelihood of or a predisposition to having a disease or condition, estimating the severity of a disease or condition, determining the risk of progression in a disease or condition, assessing the clinical response to a treatment, and/or predicting the response to treatment.

A “biomarker,” as used herein, is any molecule or compound that is found in a sample of interest and that is known to be diagnostic of or associated with the presence of or a predisposition to a disease or condition of interest in the subject from which the sample is derived. Biomarkers include, but are not limited to, polypeptides or a complex thereof (e.g., antigen, antibody), nucleic acids (e.g., DNA, miRNA, mRNA), drug metabolites, lipids, carbohydrates, hormones, vitamins, etc., that are known to be associated with a disease or condition of interest.

A “condition” as used herein with respect to diagnosing a health condition, refers to a physiological state of mind or body that is distinguishable from other physiological states. A health condition may not be diagnosed as a disease in some cases. Exemplary health conditions of interest include, but are not limited to, nutritional health; aging; exposure to environmental toxins, pesticides, herbicides, synthetic hormone analogs; pregnancy; menopause; andropause; sleep; stress; prediabetes; exercise; fatigue; chemical balance; etc. The term “biotin moiety” refers to an affinity agent that includes biotin or a biotin analogue such as desthiobiotin, oxybiotin, 2′-iminobiotin, diaminobiotin, biotin sulfoxide, biocytin, etc. Biotin moieties bind to streptavidin with an affinity of at least 10-8M. A biotin affinity agent may also include a linker, e.g., -LC-biotin, -LC-LC-Biotin, -SLC-Biotin or -PEGn-Biotin where n is 3-12.

The term “streptavidin” refers to both streptavidin and avidin, as well as any variants thereof that bind to biotin with high affinity.

The term “marker”, as used in describing a biological sample, refers to an analyte whose presence or abundance in a biological sample is correlated with a disease or condition.

The term “bond” includes covalent and non-covalent bonds, including hydrogen bonds, ionic bonds and bonds produced by van der Waal forces.

The term “amplify” refers to an increase in the magnitude of a signal, e.g., at least a 10-fold increase, at least a 100-fold increase at least a 1,000-fold increase, at least a 10,000-fold increase, or at least a 100,000-fold increase in a signal.

The term “entity” refers to, but not limited to proteins, peptides, DNA, RNA, nucleic acid, molecules (small or large), cells, tissues, viruses, nanoparticles with different shapes, that would bind to a “binding site”. The entity includes the capture agent, detection agent, and blocking agent. The “entity” includes the “analyte”, and the two terms are used interchangeably.

The term “binding site” refers to a location on a solid surface that can immobilize “entity” in a sample.

The term “entity partners” refers to, but not limited to proteins, peptides, DNA, RNA, nucleic acid, molecules (small or large), cells, tissues, viruses, nanoparticles with different shapes, that are on a “binding site” and would bind to the entity. The entity, include, but not limited to, capture agents, detection agents, secondary detection agents, or “capture agent/analyte complex”.

The term “target analytes” or “target entity” refers to a particular analyte that will be specifically analyzed (i.e. detected), or a particular entity that will be specifically bound to the binding site.

The term “smart phone” or “mobile phone”, which are used interchangeably, refers to the type of phones that has a camera and communication hardware and software that can take an image using the camera, manipulate the image taken by the camera, and communicate data to a remote place. In some embodiments, the Smart Phone has a flash light.

The term “light” refers to, unless specifically specified, an electromagnetic radiation with various wavelength.

The term “average linear dimension” of an area is defined as a length that equals to the area times 4 then divided by the perimeter of the area. For example, the area is a rectangle, that has width w, and length L, then the average of the linear dimension of the rectangle is 4*W*L/(2*(L+W)) (where “*” means multiply and “/” means divide). By this definition, the average line dimension is, respectively, W for a square of a width W, and d for a circle with a diameter d. The area include, but not limited to, the area of a binding site or a storage site.

The term “period” of periodic structure array refers to the distance from the center of a structure to the center of the nearest neighboring identical structure.

The term “storage site” refers to a site of an area on a plate, wherein the site contains reagents to be added into a sample, and the reagents are capable of being dissolving into the sample that is in contract with the reagents and diffusing in the sample.

The term “relevant” means that it is relevant to detection of analytes, quantification and/or control of analyte or entity in a sample or on a plate, or quantification or control of reagent to be added to a sample or a plate.

The term “hydrophilic”, “wetting”, or “wet” of a surface means that the contact angle of a sample on the surface is less than 90 degree.

The term “hydrophobic”, “non-wetting”, or “does not wet” of a surface means that the contact angle of a sample on the surface is equal to or larger than 90 degrees.

The term “variation” of a quantity refers to the difference between the actual value and the desired value or the average of the quantity. And the term “relative variation” of a quantity refers to the ratio of the variation to the desired value or the average of the quantity. For example, if the desired value of a quantity is Q and the actual value is (Q+□), then the □ is the variation and the □/(Q+□) is the relative variation. The term “relative sample thickness variation” refers to the ratio of the sample thickness variation to the average sample thickness.

The term “optical transparent” refers to a material that allows a transmission of an optical signal, wherein the term “optical signal” refers to, unless specified otherwise, the optical signal that is used to probe a property of the sample, the plate, the spacers, the scale-marks, any structures used, or any combinations of thereof.

The term “none-sample-volume” refers to, at a closed configuration of a CROF process, the volume between the plates that is occupied not by the sample but by other objects that are not the sample. The objects include, but not limited to, spacers, air bubbles, dusts, or any combinations of thereof. Often none-sample-volume(s) is mixed inside the sample.

The term “saturation incubation time” refers to the time needed for the binding between two types of molecules (e.g. capture agents and analytes) to reach an equilibrium. For a surface immobilization assay, the “saturation incubation time” refers the time needed for the binding between the target analyte (entity) in the sample and the binding site on plate surface reaches an equilibrium, namely, the time after which the average number of the target molecules (the entity) captured and immobilized by the binding site is statistically nearly constant.

In some cases, the “analyte” and “binding entity” and “entity” are interchangeable.

A “processor,” “communication device,” “mobile device,” refer to computer systems that contain basic electronic elements (including one or more of a memory, input-output interface, central processing unit, instructions, network interface, power source, etc.) to perform computational tasks. The computer system may be a general purpose computer that contains instructions to perform a specific task, or may be a special-purpose computer.

A “site” or “location” as used in describing signal or data communication refers to the local area in which a device or subject resides. A site may refer to a room within a building structure, such as a hospital, or a smaller geographically defined area within a larger geographically defined area. A remote site or remote location, with reference to a first site that is remote from a second site, is a first site that is physically separated from the second site by distance and/or by physical obstruction. The remote site may be a first site that is in a separate room from the second site in a building structure, a first site that is in a different building structure from the second site, a first site that is in a different city from the second site, etc.

As used herein, “raw data” includes signals and direct read-outs from sensors, cameras, and other components and instruments which detect or measure properties or characteristics of a sample. For example, raw data includes voltage or current output from a sensor, detector, counter, camera, or other component or device; raw data includes digital or analog numerical output from a sensor, detector, counter, camera, or other component or device; and raw data may include digitized or filtered output from a sensor, detector, counter, camera, or other component or device. For example, raw data includes the output of a luminometer, which may include output in “relative light units” which are related to the number of photons detected by the luminometer. Raw data may include a JPEG, bitmap, or other image file produced by a camera. Raw data may include cell counts; light intensity (at a particular wavelength, or at or within a range of wavelengths); a rate of change of the output of a detector; a difference between similar measurements made at two times; a number of events detected; the number of events detected within a pre-set range or that meet a pre-set criterion; the minimum value measured within a time period, or within a field of view; the maximum value measured within a time period, or within a field of view; and other data. Where sufficient, raw data may be used without further processing or analysis. In other cases, raw data may be further processed or used for further analysis related to the sample, the subject, or for other purposes.

“Representative of a sample” as used in reference to an output signal or raw data that are representative of the sample, refers to the output signal or raw data reflecting a measured property of the sample or a portion thereof, e.g., reflecting the amount of analyte of interest present in the sample. For instance, the intensity of a fluorescence signal representative of a sample may be more intense in a fluorescently labeled sample that contains more analyte of interest than the intensity of a fluorescence signal representative of a fluorescently labeled sample that contains less analyte.

The term “compressed open flow (COF)” refers to a method that changes the shape of a flowable sample deposited on a plate by (i) placing other plate on top of at least a part of the sample and (ii) then compressing the sample between two plates by pushing the two plates towards each other; wherein the compression reduces a thickness of at least a part of the sample and makes the sample flow into open spaces between the plates.

The term “compressed regulated open flow” or “CROF” (or “self-calibrated compressed open flow” or “SCOF” or “SCCOF”) refers to a particular type of COF, wherein the final thickness of a part or entire sample after the compression is “regulated” by spacers, wherein the spacers, that are placed between the two plates.

The term “the final thickness of a part or entire sample is regulated by spacers” in a CROF means that during a CROF, once a specific sample thickness is reached, the relative movement of the two plates and hence the change of sample thickness stop, wherein the specific thickness is determined by the spacer.

The practice of various embodiments of the present disclosure employs, unless otherwise indicated, conventional techniques of immunology, biochemistry, chemistry, molecular biology, microbiology, cell biology, genomics and recombinant DNA, which are within the skill of the art. See Green and Sambrook, MOLECULAR CLONING: A LABORATORY MANUAL, 4th edition (2012); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

One with skill in the art will appreciate that the present invention is not limited in its application to the details of construction, the arrangements of components, category selections, weightings, pre-determined signal limits, or the steps set forth in the description or drawings herein. The invention is capable of other embodiments and of being practiced or being carried out in many different ways.

### Principles and Certain Examples

One objective of the present invention is to design a passive illuminator for illuminating an assaying device. The term “assaying device” is defined as a device used for assaying. Another objective of the present invention is to design the passive illuminator in an adapter for using with a smartphone. Another objective of the present invention is to generate diffusive light for illuminating an assaying device.

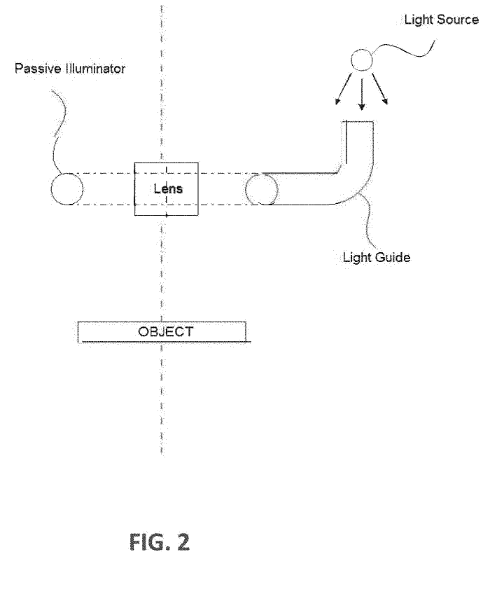

FIG. 2 shows a passive illuminator that is positioned around the outside peripheral of an imager lens in accordance with some embodiment. In FIG. 2, light from a light source impinges upon one or two light-guides each connecting to one end of the passive illuminator. The impinging light travels through each light-guide to reach the corresponding end of the passive illuminator. After the impinging light enters the corresponding end of the passive illuminator, it causes light to be emitted from a side wall of the passive illuminator. Illumination light is generated from the light emitted from the side wall of the passive illuminator for illuminating an object positioned in front of the imager lens. In some implementations, the light-guides and the passive illuminator are jointly formed by an optical fiber.

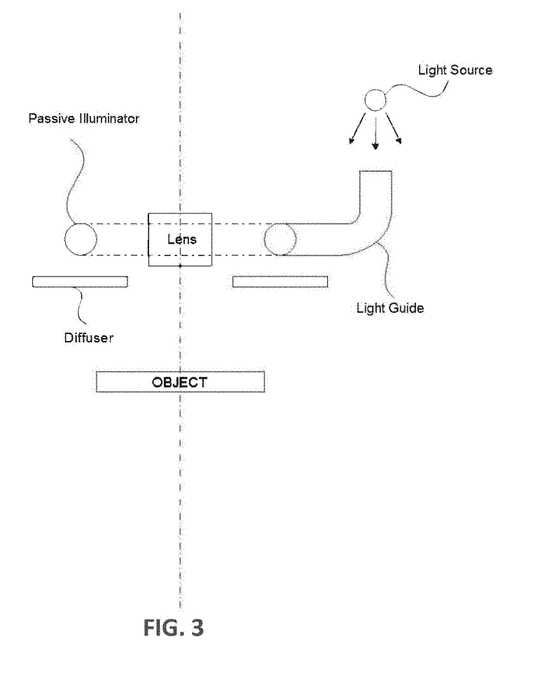

FIG. 3 shows a passive illuminator and a diffuser that are positioned around the outside peripheral of an imager lens in accordance with some embodiment. In FIG. 3, light from a light source impinges upon one or two light-guides each connecting to one end of the passive illuminator. The impinging light travels through each light-guide to reach the corresponding end of the passive illuminator. After the impinging light enters the corresponding end of the passive illuminator, it causes light to be emitted from a side wall of the passive illuminator. The light emitted from the side wall of the passive illuminator passes through a diffuser and generates Illumination light for illuminating an object positioned in front of the imager lens. In some implementations, the light-guides and the passive illuminator are jointly formed by an optical fiber.

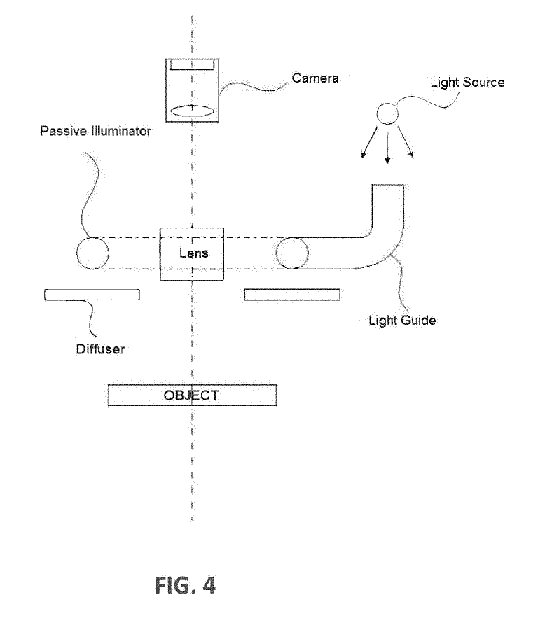

FIG. 4 shows a passive illuminator and a diffuser that are positioned around the outside peripheral of an imager lens in accordance with some embodiment. In FIG. 4, light from a light source impinges upon one or two light-guides each connecting to one end of the passive illuminator. The impinging light travels through each light-guide to reach the corresponding end of the passive illuminator. After the impinging light enters the corresponding end of the passive illuminator, it causes light to be emitted from a side wall of the passive illuminator. The light emitted from the side wall of the passive illuminator passes through a diffuser and generates Illumination light for illuminating an object positioned in front of the imager lens. The object illuminated by the Illumination light can be imaged by a camera though the imager lens. In some implementations, the light-guides and the passive illuminator are jointly formed by an optical fiber.

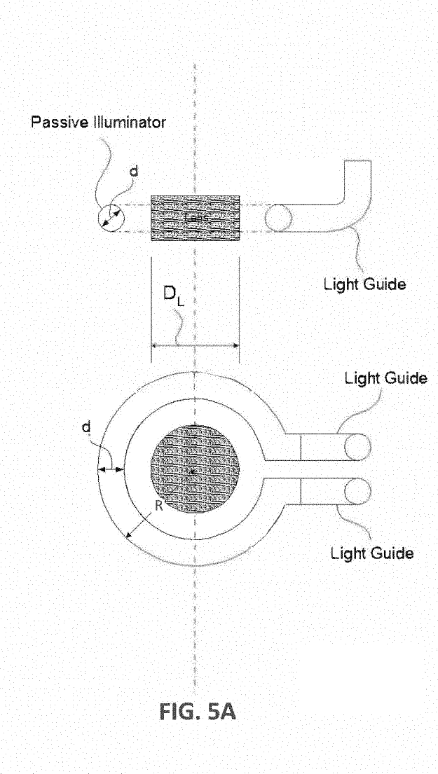

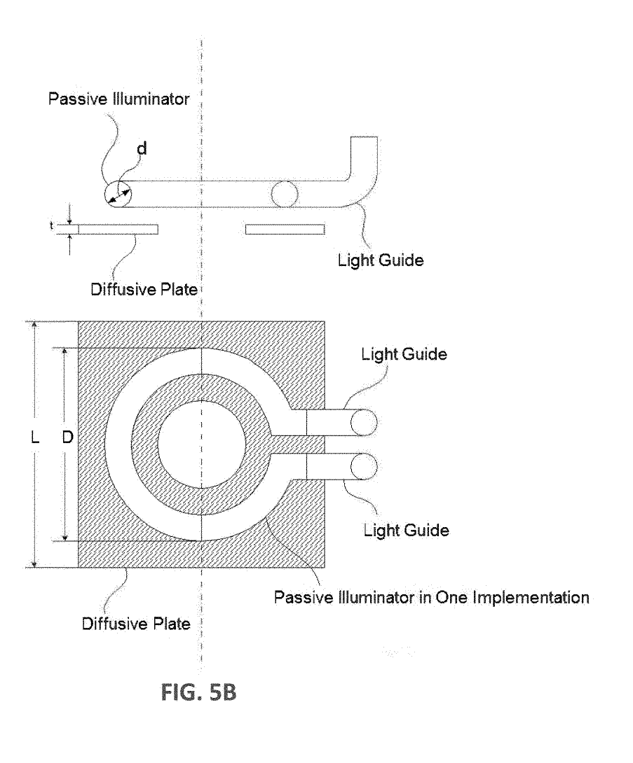

FIG. 5A shows one implementation of a passive illuminator that are positioned around the outside peripheral of an imager lens in accordance with some embodiment. In some embodiments, the passive illuminator can be formed by an optical fiber in the form of a ring that is in the shape of a circle with a diameter D. This diameter D can take any value between 5 mm and 100 mm. In some embodiments, the optical fiber for forming the passive illuminator can have a substantially identical cross-section. Such cross-section can be in the form of a circle with a diameter d. This diameter d can take any value between 0.5 mm and 10 mm. The imager lens has a diameter D_{L}, which can take any value between 2 mm and 50 mm. In some embodiments of FIG. 5A, light can enter the optical fiber ring from one end of the optical fiber. In some other embodiments of FIG. 5A, light can enter the optical fiber ring from both ends of the optical fiber.

Depending upon the implementations or designs, the diameter D of the circle formed by the optical fiber ring can be at least 5 mm, 10 mm, 15 mm, 20 mm, 25 mm, 30 mm, 40 mm, 50 mm, 60 mm, 80 mm, or 100 mm, or in a range between any of the two values. Depending upon the implementations or designs, the diameter of the circle formed by the cross-section of the optical fiber can be at least 0.5 mm, 1.0 mm, 1.5 mm, 2.0 mm, 2.5 mm, 3 mm, 4 mm, 5 mm, 6 mm, 8 mm, or 10 mm, or in a range between any of the two values. Depending upon the implementations or designs, the diameter D_{L} of the imager lens can be at least 2 mm, 3 mm, 4 mm, 5 mm, 10 mm, 15 mm, 20 mm, 25 mm, 30 mm, 40 mm, or 50 mm, or in a range between any of the two values.

FIG. 5B shows one implementation of a passive illuminator and a diffuser that are positioned around the outside peripheral of an imager lens in accordance with some embodiment. In FIG. 5B, the passive illuminator is implemented as a ring that has two ends each connecting to a corresponding end of a light guide. In FIG. 5B, the diffuser is implemented as a diffusive plate that has an opening in its inner area to allow an object be imaged by a camera through such opening and the imager lens. In some embodiments, the diffuser can be implemented as a diffusive plate that is in the shape of a rectangular or a square. One side of the rectangular or the square has a length L. This length L can be any value between 5 mm and 200 mm. The diffusive plate can have a thickness t that can take any value between in a range of 2 mm to 20 mm.

Depending upon the implementations or designs, the length L of one side of the diffusive plate can be at least 5 mm, 10 mm, 15 mm, 20 mm, 25 mm, 30 mm, 40 mm, 50 mm, 100 mm, 150 mm, or 200 mm, or in a range between any of the two values. Depending upon the implementations or designs, the thickness t of the diffusive plate can be at least 2 mm, 3 mm, 4 mm, 5 mm, 10 mm, 15 mm, or 20 mm, or in a range between any of the two values.

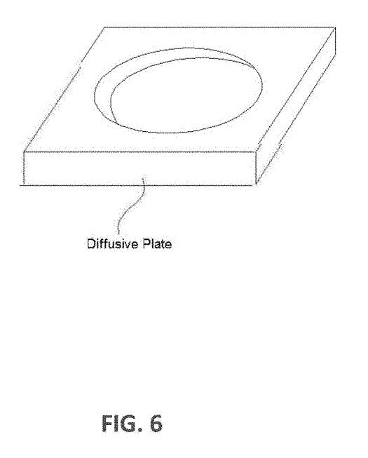

FIG. 6 is a three-dimensional view of the diffusive plate in FIG. 5B. The diffusive plate can be made of a volume diffusive material. In some implementations, either the upper surface or the lower surface of the diffusive plate can be in the form of diffusive textured surface. In some implementations, both the upper surface and the lower surface of the diffusive plate can be in the form of diffusive textured surface.

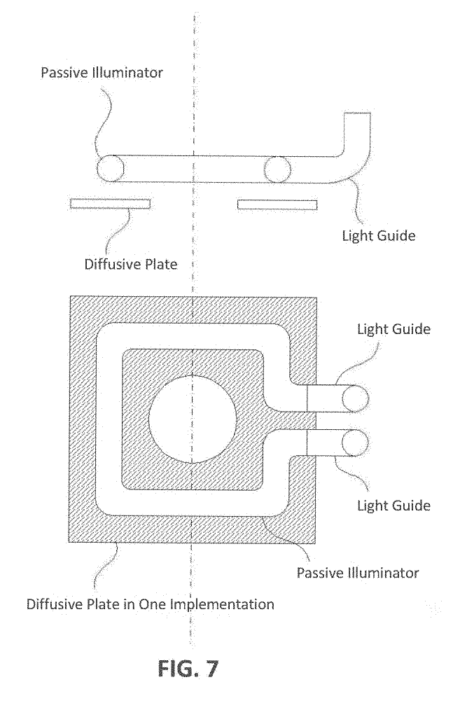

FIG. 7 shows another implementation of a passive illuminator and a diffuser that are positioned around the outside peripheral of an imager lens in accordance with some embodiment. In FIG. 7, the passive illuminator is implemented as a ring that has two ends each connecting to a corresponding end of a light guide. The passive illuminator is in a shape somewhat like a quadrilateral. The diffusive plate has an opening in its inner area to allow an object be imaged by a camera through such opening and the imager lens.

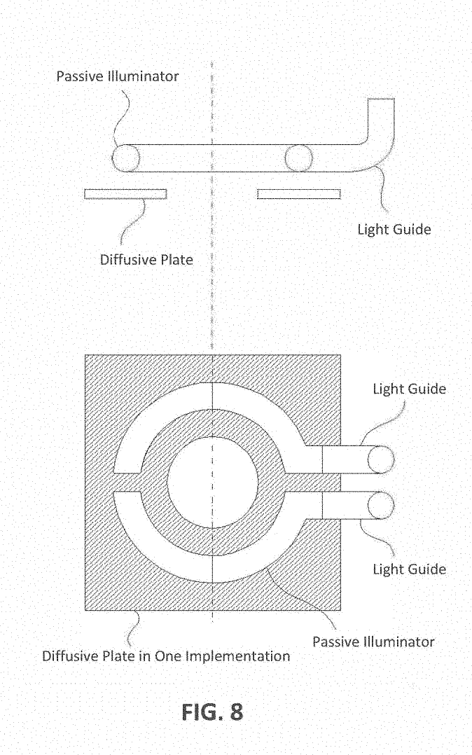

FIG. 8 shows one implementation of a passive illuminator and a diffuser that are positioned around the outside peripheral of an imager lens in accordance with some embodiment. In FIG. 8, the passive illuminator is implemented as a broken ring that has two ends each connecting to a corresponding end of a light guide. The passive illuminator is in a shape somewhat like a circle. The diffusive plate has an opening in its inner area to allow an object be imaged by a camera through such opening and the imager lens.

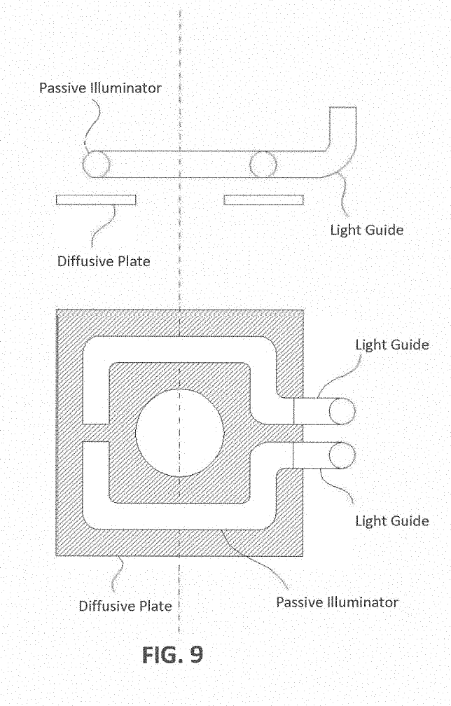

FIG. 9 shows another implementation of a passive illuminator and a diffuser that are positioned around the outside peripheral of an imager lens in accordance with some embodiment. In FIG. 9, the passive illuminator is implemented as a broken ring that has two ends each connecting to a corresponding end of a light guide. The passive illuminator is in a shape somewhat like a quadrilateral. The diffusive plate has an opening in its inner area to allow an object be imaged by a camera through such opening and the imager lens.

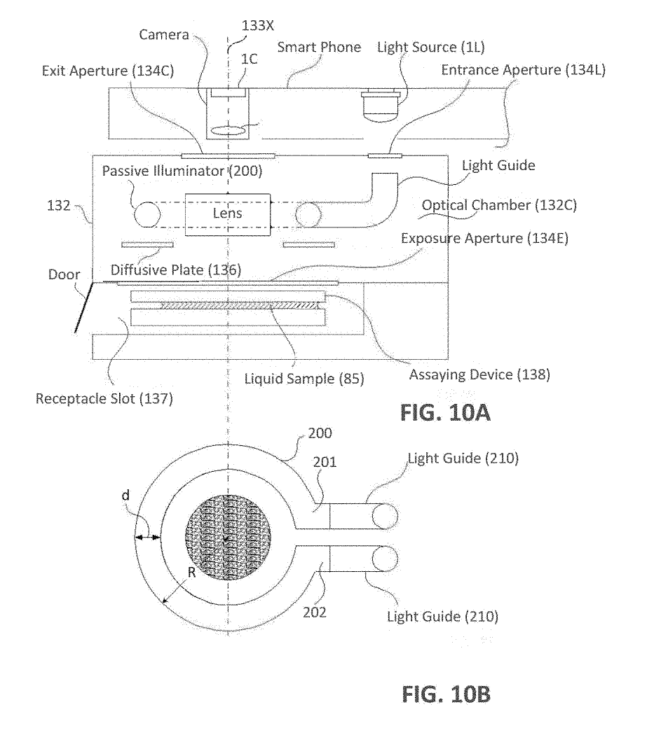

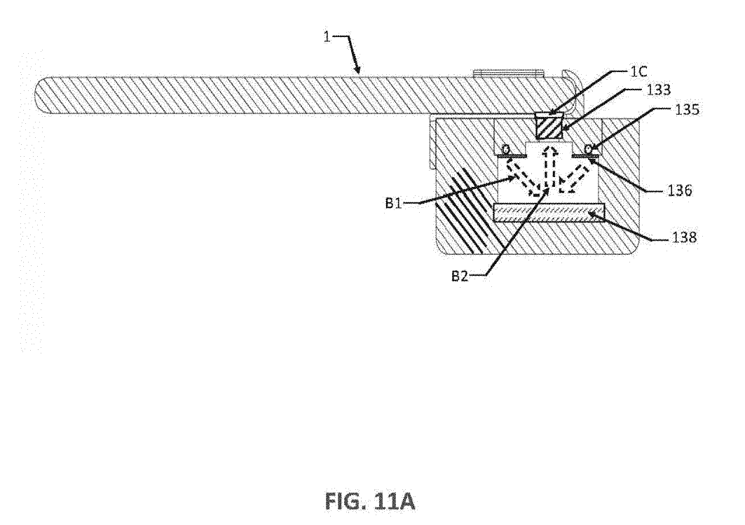

FIG. 10A is a schematic of an adaptor for using with a smartphone to read an assaying device in accordance with some embodiment. In FIG. 10A, the adaptor includes an optical chamber that has an entrance aperture, an exit aperture, and an exposure aperture. The light guide, the passive illuminator, the imaging lens, and the diffuser that are positioned inside the optical chamber. The adaptor includes a receptacle slot for holding the assaying device. The entrance aperture and the exit aperture are aligned respectively with the camera and the light source of the smartphone when the adaptor is engaged with the smartphone. The exposure aperture is aligned with the exit aperture for exposing optically at least part of a plate in the assaying device to the exit aperture of the optics chamber through the exposure aperture of the optics chamber when the assay device is inserted into the receptacle slot. Each of the entrance aperture, the exit aperture, and the exposure aperture can be covered with windows to prevent dirt or debris from damaging any optical components in the optical chamber.

FIG. 10B is a schematic of a passive illuminator 200 in the optical adaptor 13 of FIG. 10A in accordance with some embodiments. In some embodiments, as shown in FIG. 10A and FIG. 10B, the passive illuminator 200 can be in the form of a ring that is configured to surround an optical axis 133x of a lens 133 in the camera of the smartphone when the optical adaptor 13 is engaged with the smartphone. The optical adaptor 13 can include at least one light-guide 210 configured to receive light from the light source 1L of the smartphone to cause the light to travel through the light-guide to reach a first end 201 of the passive illuminator 200 when the optical adaptor 13 is engaged with the smartphone. The optical adaptor 13 can include a receptacle slot 137 to hold the assay device 138 for exposing at least part of a plate in the assay device 138 to the lens 133 in the camera 1C when the optical adaptor 13 is engaged with the smartphone. In some embodiments, the light-guide 210 and the passive illuminator 200 are jointly formed by an optical fiber 135.

In some embodiments, the optical adaptor 13 can include two light-guides 210 and 220 each receiving light from the light source 1L of the smartphone when the optical adaptor 13 is engaged with the smartphone. Light received from the light source 1L travels through the light-guide 210 to reach the corresponding end 201 of the passive illuminator 200. Light received from the light source 1L travels through the light-guide 220 to reach the corresponding end 202 of the passive illuminator 200. In some embodiments, the two light-guides 210 and 220 and the passive illuminator 200 are jointly formed by an optical fiber 135.

In some embodiments, the optical adaptor 13 includes an optics chamber 132C that as an entrance aperture 134L and an exit aperture 134C at a first side of the optics chamber 132C and having an exposure aperture 134E at a second side of the optics chamber 132C. The first side of the optics chamber 132C is the side near the smartphone, the second side of the optics chamber 132C is the side near the receptacle slot 137. The exposure aperture 134E allows part of a first plate in the assay device 138 be optically exposed to the camera 1C through the exposure aperture 134E and the exit aperture 134C of the optics chamber 132C.

In some embodiments, the passive illuminator 200 is formed by a side illumination optical fiber. An optical fiber has a high-refractive-index core and a low-refractive-index cladding layer. For a conventional end-emitting fiber, the light propagates in the core and is trapped by the total internal reflection at the core/cladding boundary. And the boundary is very efficient and total internal reflectivity is close to 100%. So, light can only come out of the fiber from the end surfaces. However, for a side-illumination fiber, the core/cladding boundary is inefficient and rough. At the boundary, a small percentage of light is scattered into the cladding layer and then into the air. In some embodiments, as shown in FIG. 10B, the passive illuminator 200 is rotationally symmetric. In some embodiments, as shown in FIG. 10B, the passive illuminator 200 is in the form of a circle. The circle having a diameter R that is in a range from 10 mm to 30 mm. In some embodiments, the passive illuminator 200 can be in the form of a convex polygon. In some embodiments, the passive illuminator 200 can be in the form of a star polygon. In some embodiments, as shown in FIGS. 10A-10B, the optical axis 133x of the lens 133 passes through a center 250 of the passive illuminator 200 when the passive illuminator 200 is in a form that is rotationally symmetric. In some embodiments, the passive illuminator 200 does not have to be rotationally symmetric. For example, the passive illuminator 200 can be in the form of an ellipse.

In some embodiments, the passive illuminator 200 is formed by a segment of optical fiber. The optical fiber generally has a substantially uniform cross-section. In other embodiments, even if the passive illuminator 200 is not formed by a segment of optical fiber, the passive illuminator 200 can still be manufactured to have a substantially uniform cross-section. In some embodiments, all of the cross-sections at locations on more than 50% length of the passive illuminator 200 are substantially identical in shape. Such uniform cross-section can be a circle or other shape. In some embodiments, the shapes of substantially all of the cross-sections can be in the form of a circle that has a diameter d in a range from 0.5 mm to 10.0 mm. Depending upon the implementations or designs, the diameter of the circle formed by the cross-section of the optical fiber can be at least 0.5 mm, 1.0 mm, 1.5 mm, 2.0 mm, 2.5 mm, 3 mm, 4 mm, 5 mm, 6 mm, 8 mm, or 10 mm, or in a range between any of the two values. In some embodiments, the shapes of substantially all the cross-sections can be in the form of an ellipse.

In some embodiments, at least a segment of the side wall of the passive illuminator 200 is formed by a diffusive surface. For example, the passive illuminator 200 can have the sidewall facing the exposure aperture 134E configured in the form of the diffusive surface. In some embodiments, the optical adaptor 13 can further include another diffuser 136, such as the diffuser 136 placed between the passive illuminator 200 and the receptacle slot 137.

In some embodiments, the optical adaptor 13 can include a diffuser 136 that is placed at a predetermined distance from the passive illuminator 200 in accordance with some embodiments. The diffuser 136 and its position is often configured to make the light illumination on the object more uniform than that without the diffuser 136 being in place. In some embodiments, when the passive illuminator 200 is substantially rotational-symmetric, the diffuser 136 can be substantially rotational-symmetric and be placed in a coaxial position with the passive illuminator 200. The diffuser 136 can have an opening 136C configured to expose to the camera of the smartphone at least a part of the exposure aperture 134E in the optics chamber 132C when the optical adaptor 13 is engaged with the smartphone. In some embodiments, the diffuser 136 can be configured to intercept all light path directly between the passive illuminator 200 and the exposure aperture 134E of the optics chamber 132C.

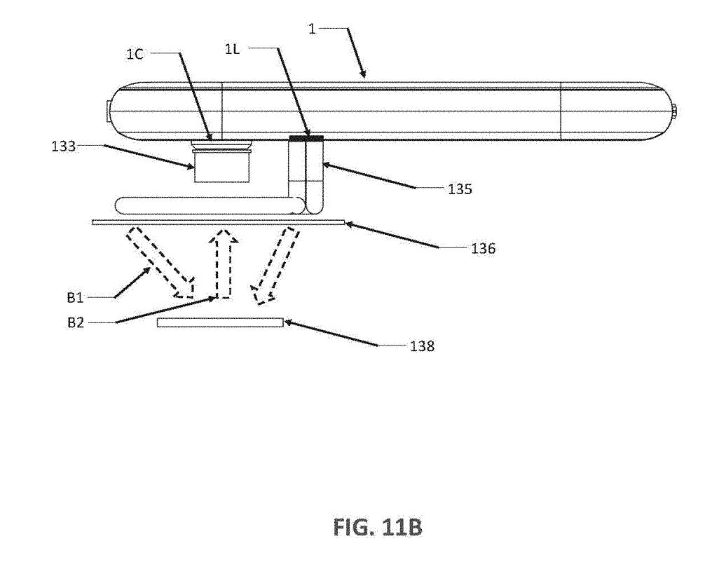

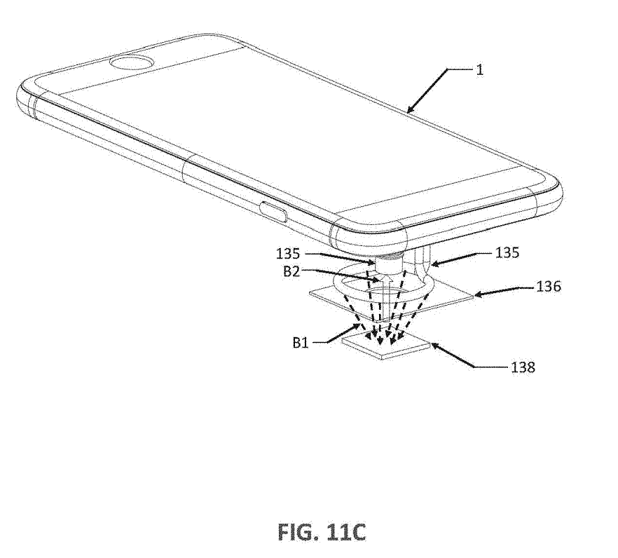

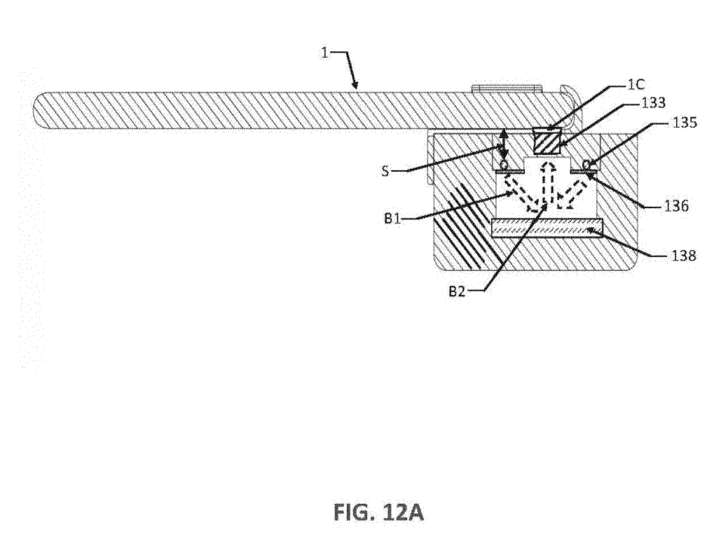

FIGS. 11A, 11B, and 11C are the schematic views showing details of system 10 reading an assaying device, and particularly of device 13. FIG. 11A is the sectional view showing details of device 13. And FIG. 11B and FIG. 11C are the schematic views only showing the configuration of the optics elements in device 13. The light emitted from light source 1L is coupled into side-emitting optical fiber ring 135 from the two end faces of fiber ring 135 and travels inside along the ring. Beam B1 is emitted out from the side wall of fiber ring and go through the diffuser film 136. Beam B1 illuminates the sample area of colorimetric sample card 138 right under the camera 1C from front side to create uniform illumination. The illuminated sample area absorbs part of beam B1 and reflects the beam B1 to beam B2. Beam B2 is collected by lens 133 and gets into camera 1C Lens 133 creates an image of the sample area on the image sensor plane of camera 1C. Smartphone 1 captures and processes the image to analyze the assay.

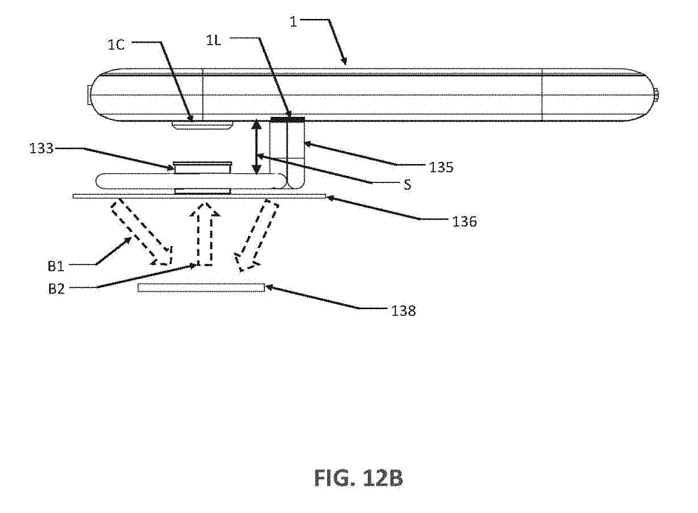

FIGS. 12A-12B shows a passive illuminator 135 that is positioned around the outside peripheral of an imager lens 133 in accordance with some embodiment. FIGS. 12A-12B shows a diffuser 136 that is configured to intercept all light path directly between the passive illuminator 135 and the assaying device 138. In FIGS. 12A-12B, the upper surface of the passive illuminator can be separated from the face of the smartphone by a distance S. This distance S can be any value between 5 mm and 50 mm. Depending upon the implementations or designs, the distance S that separates the passive illuminator from the face of the smartphone can be at least at least 5 mm, 10 mm, 15 mm, 20 mm, 25 mm, 30 mm, 40 mm, or 50 mm, or in a range between any of the two values.

### Assays, Capture Agent, and Detection Agent

In some embodiments, the assay is a sandwich assay, in which capture agent and detection agent are configured to bind to analyte at different locations thereof, forming capture agent-analyte-detection agent sandwich.

In some embodiments, the assay is a competitive assay, in which analyte and detection agent compete with each other to bind to the capture agent.

In some embodiments, the assay is an immunoassay, in which protein analyte is detected by antibody-antigen interaction. In some embodiments, the assay is a nucleic acid assay, in which nucleic acids (e.g. DNA or RNA) are detected by hybridization with complementary oligonucleotide probes.

In some embodiments, the assay utilizes light signals as readout. In some embodiments, the assay utilizes magnetic signals as readout. In some embodiments, the assay utilizes electric signals as readout. In some embodiments, the assay utilizes signals in any other form as readout.

In some embodiments, the light signal from the assay is luminescence selected from photoluminescence, electroluminescence, and electrochemiluminescence. In some embodiments, the light signal is light absorption, reflection, transmission, diffraction, scattering, or diffusion. In some embodiments, the light signal is surface Raman scattering. In some embodiments, the electrical signal is electrical impedance selected from resistance, capacitance, and inductance. In some embodiments, the magnetic signal is magnetic relaxivity. In some embodiments, the signal is any combination of the foregoing signal forms.

There are many examples of analyte concentration surfaces that capture analyte using a capture agent, and the captured analyte are further bound with a label. As a first example, a protein concentration surface can be coated with capture antibodies. The capture antibodies capture the protein analyte in a sample, which is further bound with labeled detection antibodies. In this case, the capture antibody and detection antibody are configured to bind to the protein analyte at its different locations, therefore forming a capture antibody-protein analyte-detection antibody sandwich. As a second example, a nucleic acid concentration surface-can be coated with oligonucleotide capture probes. The capture probes are complementary to one part of the nucleic acid analyte, therefore capturing the analyte to the surface. Further, the analyte is bound with a labeled detection probe that is complementary to another part of the analyte. As a third example, protein analyte can be directly labeled by an optical label and captured by the capture antibodies that are coated on the concentration surface. As a fourth example, protein analyte can be bound with a quencher, which quenches the signal emitted by the label that is associated with the capture antibodies on the concentration surface. In this case, the concentration of the protein analyte to the concentration surface reduces the signal emanating from the concentration surface.

In some embodiments, the capture agent and the detection agent are configured to bind to the analyte at different locations thereof and to form a capture agent-analyte-detection agent sandwich that is immobilized to the separated nano-/micro-islands on one or both of the plates; wherein the shape of nano- or micro-islands are selected from the group consisting of sphere, rectangle, hexagon, and/or any other polyhedron, with lattice of square, hexagon, and/or any other lattices.

In some embodiments, the material of protrusions that are nano or micro islands are selected from the group consisting of plastic as polystyrene, polypropylene, polycarbonate, PMMA, PET; metals as gold, aluminum, silver, copper, tin and/or their combinations; or any other material whose surface can be modified to be associated with the capture agent.

As discussed above, in some embodiments, the beads, the capture agent, and the detection agent are configured to render signal of the bead-captured analyte distinguishable from signal of free detection agent in the layer of uniform thickness. In some embodiments, it is critical to achieve the foregoing configuration, in that only if the signal from the sandwich structure is distinguishable from the “background” signal of the free detection agent in the layer of uniform thickness, one can use the detected signals as a readout of the presence and/or quantity of the analyte in the sample, thereby realizing the assay.

In some embodiments, the target analyte competes with the detection agent on the capture locations on beads. When more target analyte appears, beads become relative dark.

In some embodiments, the beads are associated with a label, and the detection agent is a quencher that is configured to quench signal of the beads-associated label when the detection agent is in proximity of the label. When beads capture the target analyte, the label on beads become quenched or dimed.

In some embodiments, the capture agent includes, but not limited to, protein, peptide, peptidomimetics, streptavidin, biotin, oligonucleotide, oligonucleotide mimetics, any other affinity ligand and any combination thereof. In some embodiments, the capture agent is an antibody. In some embodiments, the capture antibody is an anti-C Reactive Protein (CRP) antibody.

In some embodiments, the capture agent has a concentration that is sufficient to detect the presence and/or measure the amount of the analyte. In some embodiments, the capture agent has a concentration that is sufficient to immobilize the analyte.

In some embodiments, the detection agent includes, but not limited to, protein, peptide, peptidomimetics, streptavidin, biotin, oligonucleotide, oligonucleotide mimetics, any other affinity ligand and any combination thereof. In some embodiments, the detection agent is an antibody. In some embodiments, the detection antibody is an anti-CRP antibody.

In some embodiments, the detection antibody is configured to have a concentration in the layer of uniform thickness that is higher than analyte concentration in the sample. In some embodiments, the ratio of the detection antibody concentration over the analyte concentration is 1 or more, 2 or more, 5 or more, 10 or more, 20 or more, 30 or more, 50 or more, 100 or more, 200 or more, 300 or more, 500 or more, 1000 or more, or in a range between any two of these values.

In some embodiments, the detection antibody is labeled. In some embodiments, the label can be fluorescent, colorimetric or luminescent. In some embodiments, the detection antibody is labeled with a fluorophore. In some embodiments, the fluorophores include, but are not limited to, IRDye800CW, Alexa 790, Dylight 800, fluorescein, fluorescein isothiocyanate, succinimidyl esters of carboxyfluorescein, succinimidyl esters of fluorescein, 5-isomer of fluorescein dichlorotriazine, caged carboxyfluorescein-alanine-carboxamide, Oregon Green 488, Oregon Green 514; Lucifer Yellow, acridine Orange, rhodamine, tetramethylrhodamine, Texas Red, propidium iodide, JC-1 (5,5′,6,6′-tetrachloro-1,1′,3,3′-tetraethylbenzimidazoylcarbocyanine iodide), tetrabromorhodamine 123, rhodamine 6G, TMRM (tetramethyl rhodamine methyl ester), TMRE (tetramethyl rhodamine ethyl ester), tetramethylrosamine, rhodamine B and 4-dimethylaminotetramethylrosamine, green fluorescent protein, blue-shifted green fluorescent protein, cyan-shifted green fluorescent protein, red-shifted green fluorescent protein, yellow-shifted green fluorescent protein, 4-acetamido-4′-isothiocyanatostilbene-2,2′disulfonic acid; acridine and derivatives, such as acridine, acridine isothiocyanate; 5-(2′-aminoethyl)aminonaphthalene-1-sulfonic acid (EDANS); 4-amino-N-[3-vinylsulfonyl)phenyl]naphth-alimide-3,5 disulfonate; N-(4-anilino-1-naphthyl)maleimide; anthranilamide; 4,4-difluoro-5-(2-thienyl)-4-bora-3a,4a diaza-5-indacene-3-propioni-c acid BODIPY; cascade blue; Brilliant Yellow; coumarin and derivatives: coumarin, 7-amino-4-methylcoumarin (AMC, Coumarin 120), 7-amino-4-trifluoromethylcoumarin (Coumarin 151); cyanine dyes; cyanosine; 4′,6-diaminidino-2-phenylindole (DAPI); 5′,5″-dibromopyrogallol-sulfonaphthalein (Bromopyrogallol Red); 7-diethylamino-3-(4′-isothiocyanatophenyl)-4-methylcoumarin; diethylenetriaamine pentaacetate; 4,4′-diisothiocyanatodihydro-stilbene-2-,2′-disulfonic acid; 4,4′-diisothiocyanatostilbene-2,2′-disulfonic acid; 5-(dimethylamino]naphthalene-1-sulfonyl chloride (DNS, dansylchloride); 4-dimethylaminophenylazophenyl-4′-isothiocyanate (DABITC); eosin and derivatives: eosin, eosin isothiocyanate, erythrosin and derivatives: erythrosin B, erythrosin, isothiocyanate; ethidium; fluorescein and derivatives: 5-carboxyfluorescein (FAM), 5-(4,6-dichlorotriazin-2-yl)amino-fluorescein (DTAF), 2′,7′dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelli-feroneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′-tetramethyl-6-carboxyrhodamine (TAMRA); tetramethyl rhodamine; tetramethyl hodamine isothiocyanate (TRITC); riboflavin; 5-(2′-aminoethyl) aminonaphthalene-1-sulfonic acid (EDANS), 4-(4′-dimethylaminophenylazo)benzoic acid (DABCYL), rosolic acid; CAL Fluor Orange 560; terbium chelate derivatives; Cy 3; Cy 5; Cy 5.5; Cy 7; IRD 700; IRD 800; La Jolla Blue; phthalo cyanine; and naphthalo cyanine, coumarins and related dyes, xanthene dyes such as rhodols, resorufins, bimanes, acridines, isoindoles, dansyl dyes, aminophthalic hydrazides such as luminol, and isoluminol derivatives, aminophthalimides, aminonaphthalimides, aminobenzofurans, aminoquinolines, dicyanohydroquinones, fluorescent europium and terbium complexes; combinations thereof, and the like. Suitable fluorescent proteins and chromogenic proteins include, but are not limited to, a green fluorescent protein (GFP), including, but not limited to, a GFP derived from Aequoria victoria or a derivative thereof, e.g., a “humanized” derivative such as Enhanced GFP; a GFP from another species such as Renilla reniformis, Renilla mulleri, or Ptilosarcus guernyi; “humanized” recombinant GFP (hrGFP); any of a variety of fluorescent and colored proteins from Anthozoan species; combinations thereof; and the like.

In some embodiments, the beads are treated with a protein stabilizer. In some embodiments, the beads can be deposited on the plate and dried (e.g. air-dried), further simplifying the process. In some embodiments, the detection antibody is placed on one of the plates and dried. In some embodiments, the plate with the detection antibody is treated with protein stabilizer. In some embodiments, the detection antibody with protein stabilizer is pre-printed on one of the plates and air dried.

In some embodiments, wherein the beads are prepared by:

(a) activating with N-Hydroxysuccinimide (NHS);

(b) blocking with a BSA solution; and

(c) incubating with a capture agent solution.

### Detector, System and Smartphone-Based System

Another aspect of the present invention provides a system for homogeneous assay. In some embodiments, the system comprises the device as discussed above and a detector that detects the analyte in the layer of uniform thickness.

In some embodiments, detector detects a signal from the capture agent-analyte-detection agent sandwich indicative of the presence and/or quantity of the analyte.

In some embodiments, the signal is:
i. luminescence selected from photoluminescence, electroluminescence, and electrochemiluminescence;
ii. light absorption, reflection, transmission, diffraction, scattering, or diffusion;
iii. surface Raman scattering;
iv. electrical impedance selected from resistance, capacitance, and inductance;
v. magnetic relaxivity; or
vi. any combination of i-v.

Another aspect of the present invention provides a smartphone system for homogeneous assay. In some embodiments, the smartphone system comprises:
(a) a device of any aforementioned embodiment;
(b) a mobile communication device that comprises:
   i. one or a plurality of cameras for detecting and/or imaging the sample;
   ii. electronics, signal processors, hardware and software for receiving and/or processing the detected signal and/or the image of the sample and for remote communication; and
(c) an adaptor configured to hold the closed device and engageable to mobile communication device;

wherein when engaged with the mobile communication device, the adaptor is configured to facilitate the detection and/or imaging of the analyte in the sample at the closed configuration.

In some embodiments, the mobile communication device is configured to communicate test results to a medical professional, a medical facility or an insurance company.

In some embodiments, the mobile communication device is further configured to communicate information on the subject with the medical professional, medical facility or insurance company.

In some embodiments, the mobile communication device is configured to receive a prescription, diagnosis or a recommendation from a medical professional.

In some embodiments, the mobile communication device communicates with the remote location via a wifi or cellular network.

In some embodiments, the mobile communication device is a mobile phone.

In some embodiments, the images can be taken by a camera that is part of a mobile device. In some embodiments, the mobile device is a smart phone.

### Analyte, Sample and Application

In some embodiments, the analyte to be detected in the homogeneous assay includes, but not limited to, cells, viruses, proteins, peptides, DNAs, RNAs, oligonucleotides, and any combination thereof.

In some embodiments, the present invention finds use in detecting biomarkers for a disease or disease state. In certain instances, the present invention finds use in detecting biomarkers for the characterization of cell signaling pathways and intracellular communication for drug discovery and vaccine development. For example, the present invention may be used to detect and/or quantify the amount of biomarkers in diseased, healthy or benign samples. In certain embodiments, the present invention finds use in detecting biomarkers for an infectious disease or disease state. In some cases, the biomarkers can be molecular biomarkers, such as but not limited to proteins, nucleic acids, carbohydrates, small molecules, and the like. The present invention find use in diagnostic assays, such as, but not limited to, the following: detecting and/or quantifying biomarkers, as described above; screening assays, where samples are tested at regular intervals for asymptomatic subjects; prognostic assays, where the presence and or quantity of a biomarker is used to predict a likely disease course; stratification assays, where a subject's response to different drug treatments can be predicted; efficacy assays, where the efficacy of a drug treatment is monitored; and the like.

The present invention has applications in (a) the detection, purification and quantification of chemical compounds or biomolecules that correlates with the stage of certain diseases, e.g., infectious and parasitic disease, injuries, cardiovascular disease, cancer, mental disorders, neuropsychiatric disorders and organic diseases, e.g., pulmonary diseases, renal diseases, (b) the detection, purification and quantification of microorganism, e.g., virus, fungus and bacteria from environment, e.g., water, soil, or biological samples, e.g., tissues, bodily fluids, (c) the detection, quantification of chemical compounds or biological samples that pose hazard to food safety or national security, e.g. toxic waste, anthrax, (d) quantification of vital parameters in medical or physiological monitor, e.g., glucose, blood oxygen level, total blood count, (e) the detection and quantification of specific DNA or RNA from biosamples, e.g., cells, viruses, bodily fluids, (f) the sequencing and comparing of genetic sequences in DNA in the chromosomes and mitochondria for genome analysis or (g) to detect reaction products, e.g., during synthesis or purification of pharmaceuticals.

In some embodiments, the liquid sample is made from a biological sample selected from the group consisting of: amniotic fluid, aqueous humour, vitreous humour, blood (e.g., whole blood, fractionated blood, plasma or serum), breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensates, sebum, semen, sputum, sweat, synovial fluid, tears, vomit, urine, and any combination thereof.

In some embodiments, the sample is an environmental liquid sample from a source selected from the group consisting of: river, lake, pond, ocean, glaciers, icebergs, rain, snow, sewage, reservoirs, tap water, or drinking water, solid samples from soil, compost, sand, rocks, concrete, wood, brick, sewage, and any combination thereof.

In some embodiments, the sample is an environmental gaseous sample from a source selected from the group consisting of: the air, underwater heat vents, industrial exhaust, vehicular exhaust, and any combination thereof.

In some embodiments, the sample is a foodstuff sample selected from the group consisting of: raw ingredients, cooked food, plant and animal sources of food, preprocessed food, and partially or fully processed food, and any combination thereof.

### EXAMPLES OF PRESENT INVENTION

In certain embodiments of the present disclosure, a device for illuminating and imaging an object can comprise an imager having a lens and a passive illuminator. In certain embodiments of the present disclosure the passive illuminator is positioned around an outside peripheral of the imager.

In certain embodiment of the present disclosure, an adaptor for illuminating and imaging an object can comprise an adaptor housing that has an exit aperture for positioning an imager, and a passive illuminator. In certain embodiment of the present disclosure the passive illuminator is on the adaptor and is positioned around an outside peripheral of the exit aperture. In certain embodiments of the present disclosure, the adaptor housing is configured to reduce ambient light outside the adaptor housing entering into the adaptor housing.

In certain embodiments of the present disclosure, an adaptor for illuminating and imaging an object can comprise an adaptor housing that has an exit aperture for positioning an imager and a passive illuminator. In certain embodiments of the present disclosure, the passive illuminator is on the adaptor and is positioned around an outside peripheral of the exit aperture. In certain embodiments of the present disclosure, the adaptor housing (i) is configured to reduce ambient light outside the adaptor housing entering into the adaptor housing, and (ii) comprises a slot for inserting a sample holder into the adaptor housing.

In certain embodiments of the present disclosure, an adaptor for illuminating and imaging an object can comprise an adaptor housing that has an exit aperture for positioning an imager and a passive illuminator. In certain embodiments of the present disclosure, the passive illuminator is positioned around the outside peripheral of the exit aperture. In certain embodiments of the present disclosure the adaptor housing (i) is configured to reduce ambient light outside the adaptor housing entering into the adaptor housing, and (ii) comprises a slot for inserting a sample holder into the adaptor housing.

In certain embodiments of the present disclosure, an apparatus for illuminating and imaging an object can comprise a mobile phone that has a camera and a light source, an adaptor housing that has an exit aperture for positioning the imager of the phone, and a passive illuminator. In certain embodiments of the present disclosure, the passive illuminator is positioned around an outside peripheral of the exit aperture. In certain embodiments of the present disclosure, the adaptor housing is configured to reduce ambient light outside the adaptor housing entering into the adaptor housing.

In certain embodiments of the present disclosure, a method for illuminating and imaging an object can comprise the steps of providing an imager and providing a passive illuminator. In certain embodiments of the present disclosure, the passive illuminator is positioned around an outside peripheral of the imager.

In certain embodiments of the present disclosure, a method for illuminating and imaging an object, can comprise the steps of providing an imager, providing a passive illuminator, and providing an adaptor housing. In certain embodiments of the present disclosure, the adaptor has an exit aperture for positioning the imager. In certain embodiments of the present disclosure, the passive illuminator is positioned around the outside peripheral of the imager.

In certain embodiments of the present disclosure, a method for illuminating and imaging an object can comprise providing an imager, providing a passive light illuminator, providing an adaptor housing, and providing a phone that has a camera and a light source. In certain embodiments of the present disclosure, the adaptor has an exit aperture for positioning the imager. In certain embodiments of the present disclosure, the passive illuminator is positioned around the outside peripheral of the imager. In certain embodiments of the present disclosure, the adaptor housing is configured to reduce ambient light outside the adaptor housing entering into the adaptor housing. In certain embodiments of the present disclosure, the adaptor housing is configured to attach to the mobile phone.

In certain embodiments of the present disclosure, a method for illuminating and imaging a liquid sample between two parallel plates in an assay device can comprise the steps of, impinging light into one or two light-guides each connecting to one end of the passive illuminator, causing the impinging light to travel through each light-guide to reach the corresponding end of the passive illuminator, causing light to be emitted from a side wall of the passive illuminator after the impinging light enters the corresponding end of the passive illuminator, generating illumination light from the light emitted from the side wall of the passive illuminator, illuminating the liquid sample through one of the parallel plates with the illumination light, and imaging the liquid sample with an imaging sensor through a lens.

In certain embodiments of the present disclosure, a method for illuminating and imaging a liquid sample between two parallel plates in an assay device can comprise the steps of impinging light into one or two light-guides each connecting to one end of the passive illuminator, causing the impinging light to travel through each light-guide to reach the corresponding end of the passive illuminator, causing light to be emitted from a side wall of the passive illuminator after the impinging light enters the corresponding end of the passive illuminator, generating illumination light from the light emitted from the side wall of the passive illuminator and passing through a diffuser, illuminating the liquid sample through one of the parallel plates with the illumination light, and imaging the liquid sample with an imaging sensor through a lens.

In certain embodiments of the present disclosure, an apparatus for reading an assay device having two parallel plates can be for use with a smartphone including a camera and a light source. In certain embodiments of the present disclosure, the apparatus can comprise one or two light-guides each having an end thereof aligned with the entrance aperture of the optics chamber to cause light entering such end of the light-guide to travel through the light-guide to reach a corresponding end of the passive illuminator, and a passive illuminator for illuminating a liquid sample between the two parallel plates in the assay device by generating diffusive light sideways from areas surrounding an optical axis of a lens in the camera of the smartphone. In certain embodiments of the present disclosure, the passive illuminator has a first end optically coupled to a second end of the light-guide to cause light received at the first end of each light-guide to travel through the light-guide to enter the first end of the passive illuminator.

In certain embodiments of the present disclosure, an apparatus for reading an assay device having two parallel plates can be for use with a smartphone including a camera and a light source. In certain embodiments of the present disclosure, the apparatus can comprise one or two light-guides each having an end thereof aligned with the entrance aperture of the optics chamber to cause light entering such end of the light-guide to travel through the light-guide to reach a corresponding end of the passive illuminator, a passive illuminator for illuminating a liquid sample between the two parallel plates in the assay device by generating diffusive light sideways from areas surrounding an optical axis of a lens in the camera of the smartphone, and a diffuser for generating diffusive light sideways from areas surrounding the optical axis of the lens in the camera of the smartphone to illuminate the liquid sample between the two parallel plates in the assay device. In certain embodiments of the present disclosure, the passive illuminator has a first end optically coupled to a second end of the light-guide to cause light received at the first end of each light-guide to travel through the light-guide to enter the first end of the passive illuminator. In certain embodiments of the present disclosure, the

In any embodiment of the present disclosure, the one or more light-guides and the passive illuminator can be jointly formed by an optical fiber.

In any embodiment of the present disclosure, the passive illuminator can be in the form of a ring configured to surround an optical axis of a lens in the camera of the smartphone when the apparatus is engaged with the smartphone.

Any embodiment of the present disclosure can further comprise an auxiliary lens having an optical axis thereof aligned with the optical axis of the lens in the camera of the smartphone when the apparatus is engaged with the smartphone.

Any embodiment of the present disclosure can further comprise an auxiliary lens having a diameter that is at least 2 mm, 3 mm, 4 mm, 5 mm, 10 mm, 15 mm, 20 mm, 25 mm, 30 mm, 40 mm, or 50 mm, or in a range between any of the two values.

Any embodiment of the present disclosure can further comprise an optical condenser configured to be placed in front of the light source of the smartphone when the apparatus is engaged with the smartphone.

In any embodiment of the present disclosure the diffuser comprises polished surfaces on both sides, a volume diffusive material which can be but not limited to opaque white glass and opaque white plastic, wherein the transmissivity of the volume diffusive material is at least 40%, 60%, 80%, 90% or in a range between any of the two values

In any embodiment of the present disclosure, the diffuser can comprise a volume transparent material and at least one textured surface. In any embodiment of the present disclosure, the volume transparent material can be but not limited to be transparent glass, transparent plastic whose transmissivity is at least 90%. In any embodiment of the present disclosure, a grit of the textured surface can be at least 100, 200, 400, 600, 800, 1000, 2000, or in a range between any of the two values

In any embodiment of the present disclosure, the diffuser can comprise a volume diffusive material and at least one textured surface. In any embodiment of the present disclosure, the volume diffusive material can be but not limited to opaque white glass and opaque white plastic. In any embodiment of the present disclosure, the transmissivity of the volume diffusive material can be at least 40%, 60%, 80%, 90% or in a range between any of the two values. In any embodiment of the present disclosure, the grit of the textured surface can be at least 100, 200, 400, 600, 800, 1000, 2000, or in a range between any of the two values.

In any embodiment of the present disclosure, the diffuser can comprise a diffusive plate that is substantially uniform in thickness.

In any embodiment of the present disclosure, the diffuser can comprises a diffusive plate including an area that has thickness that is larger than an average thickness of the diffusive plate.

Any embodiment of the present disclosure can further a reflector configured to reflect light emitted from the passive illuminator towards the diffuser.

Any embodiment of the present disclosure can further comprises a receptacle slot operative to hold the assay device while exposing at least part of a first one of the two parallel plate in the assay device to a lens in the camera of the smartphone when the assay device is inserted into the receptacle slot and the apparatus is engaged with the smartphone. In any embodiment of the present disclosure, the light-guide has the first end configured to receive light from the light source of the smartphone when the apparatus is engaged with the smartphone.

Any embodiment of the present disclosure can comprise an optics chamber having an entrance aperture and an exit aperture at a first side of the optics chamber and having an exposure aperture at a second side of the optics chamber. In any embodiment of the present disclosure, the light-guide has the first end aligned with the entrance aperture of the optics chamber.

In any embodiment of the present disclosure, each of the entrance aperture, the exit aperture, and the exposure aperture can be covered with a window.

Any embodiment of the present disclosure can further comprise a receptacle slot operative to hold the assay device while exposing at least part of a first one of the two parallel plate in the assay device to the exposure aperture of the optics chamber when the assay device is inserted into the receptacle slot.

Any embodiment of the present disclosure can further comprise a receptacle slot has two side walls forming a cavity for holding the assay device therein. In any embodiment of the present disclosure, one of the two side walls can have an opening for forming the exposure aperture of the optics chamber.

In any embodiment of the present disclosure, the exit aperture at the first side of the optics chamber can be aligned with the exposure aperture at the second side of the optics chamber for exposing optically at least part of the first one of the two parallel plate in the assay device to the exit aperture of the optics chamber through the exposure aperture of the optics chamber when the assay device is inserted into the receptacle slot.

Any embodiment of the present disclosure can further comprise an auxiliary lens aligned with the exit aperture of the optics chamber.

In any embodiment of the present disclosure, the passive illuminator can be located between the auxiliary lens and the exit aperture of the optics chamber.

In any embodiment of the present disclosure, the auxiliary lens can be located between the passive illuminator and the receptacle slot operative to hold the assay device.

In any embodiment of the present disclosure, the auxiliary lens has an optical axis thereof coaxially aligned with an optical axis of the lens in the camera of the smartphone when the apparatus is engaged with the smartphone.

Any embodiment of the present disclosure can further comprise an optical condenser aligned with the entrance aperture of the optics chamber.

Any embodiment of the present disclosure can further comprise a reflector configured to reflect light emitted from the passive illuminator towards the exposure aperture of the optics chamber.

Any embodiment of the present disclosure, can further comprise a diffuser placed at a predetermined distance from the passive illuminator and having an opening thereof configured to expose to the camera of the smartphone at least a part of the exposure aperture in the optics chamber when the apparatus is engaged with the smartphone.

In any embodiment of the present disclosure, the diffuser can be configured to intercept all light path directly between the passive illuminator and the exposure aperture of the optics chamber.

In any embodiment of the present disclosure the imager can comprise a lens and an imaging sensor.

In any embodiment of the present disclosure, the one or two light-guides and the passive illuminator can be within an adaptor that is configured to be mounted or dismounted on the mobile phone by human hands.

In any embodiment of the present disclosure, the distance between the passive illuminator and the peripheral of the imager is in a range of 2 mm to 50 mm.

In any embodiment of the present disclosure, the distance between the passive illuminator and the peripheral of the imager can be at least 2 mm, 5 mm, 10 mm, 15 mm, 20 mm, 25 mm, 30 mm, 40 mm, or 50 mm, or in a range between any of the two values.

In any embodiment of the present disclosure, the passive illuminator can be formed by a side illumination fiber.

In any embodiment of the present disclosure, the passive illuminator can be formed by a side illumination fiber, wherein the side illumination fiber comprises a core and a cladding layer; wherein the ratio of transmissivity to reflectivity at the interface between the core and cladding layer is at least 1:100, 1:10, 1:1, or in a range between any of the two values.

In any embodiment of the present disclosure, the passive illuminator can be formed by a side illumination fiber, wherein the side illumination fiber is made of but not limited to flexible polymers, plastic, glass and rigid dielectric materials.

In any embodiment of the present disclosure, the passive illuminator can be rotationally symmetric.

In any embodiment of the present disclosure, the passive illuminator can be in the form of a circle.

In any embodiment of the present disclosure, the passive illuminator can be in the form of a circle having a diameter thereof in a range between 5 mm and 100 mm.

In any embodiment of the present disclosure, the passive illuminator can be in the form of a circle having a diameter that is at least 5 mm, 10 mm, 15 mm, 20 mm, 25 mm, 30 mm, 40 mm, 50 mm, 60 mm, 80 mm, or 100 mm, or in a range between any of the two values.

In any embodiment of the present disclosure, the passive illuminator can be in the form of a convex polygon.

In any embodiment of the present disclosure, the passive illuminator can be in the form of a star polygon.

In any embodiment of the present disclosure, the passive illuminator can be formed by a single piece of side illumination fiber.

In any embodiment of the present disclosure, the passive illuminator can a broken ring, formed by at least two segments of side illumination fibers.

In any embodiment of the present disclosure, the optical axis of the lens can pass through a center of the passive illuminator.

In any embodiment of the present disclosure, the passive illuminator can be rotationally non-symmetric.

In any embodiment of the present disclosure, the passive illuminator can be in the form of an ellipse.

In any embodiment of the present disclosure, the passive illuminator can have a substantially uniform cross-section.

In any embodiment of the present disclosure, all of the cross-sections at locations on more than 50% length of the passive illuminator can be substantially identical in shape.

In any embodiment of the present disclosure, the shapes of substantially all of the cross-sections can be in the form of a circle.

In any embodiment of the present disclosure, the shapes of substantially all the cross-sections are in the form of a circle having a diameter thereof in a range between 1.0 mm and 3.0 mm.

In any embodiment of the present disclosure, the shapes of substantially all of the cross-sections can be in the form of a circle having a diameter that is at least 0.5 mm, 1.0 mm, 1.5 mm, 2.0 mm, 2.5 mm, 3 mm, 4 mm, 5 mm, 6 mm, 8 mm, or 10 mm, or in a range between any of the two values.

In any embodiment of the present disclosure, the shapes of substantially all of the cross-sections can be in the form of an ellipse.

In any embodiment of the present disclosure, at least a segment of the side wall of the passive illuminator can be formed by a diffusive surface.

### Sample Types:

The apparatus, kit, or method of any prior embodiments, wherein the sample is original, diluted, or processed forms of: bodily fluids, stool, amniotic fluid, aqueous humour, vitreous humour, blood, whole blood, fractionated blood, plasma, serum, breast milk, cerebrospinal fluid, cerumen, chyle, chime, endolymph, perilymph, feces, gastric acid, gastric juice, lymph, mucus, nasal drainage, phlegm, pericardial fluid, peritoneal fluid, pleural fluid, pus, rheum, saliva, sebum, semen, sputum, sweat, synovial fluid, tears, vomit, urine, or exhaled breath condensate.

The apparatus, kit, or method of any prior embodiments, wherein the sample is original, diluted, or processed forms of blood.

The apparatus, kit, or method of any prior embodiments, wherein the sample comprises whole blood.

The device, method, or system of any prior embodiments, wherein the sample is a biological sample, a chemical sample, an environmental sample, or a foodstuff sample.

### Analytes:

The apparatus, kit, or method of any prior embodiments, wherein the analyte is a biomarker, an environmental marker, or a foodstuff marker.

The apparatus, kit, or method of any prior embodiments, wherein the analyte is a biomarker indicative of the presence or severity of a disease or condition.

The apparatus, kit, or method of any prior embodiments, wherein the analyte is a cell, a protein, or a nucleic acid.

The apparatus, kit, or method of any prior embodiments, wherein the analyte comprises proteins, peptides, nucleic acids, synthetic compounds, inorganic compounds, organic compounds, bacteria, virus, cells, tissues, nanoparticles, and other molecules, compounds, mixtures and substances thereof.

The apparatus, kit, or method of any prior embodiments, wherein the analyte is selected from Table B1, B2, B3 or B7 of PCT Application No. PCT/US2016/054025.

### Sample Holder:

The apparatus, kit, or method of any prior embodiments, wherein the sample holder comprises wells that configured to hold the sample.

The apparatus, kit, or method of any prior embodiments, wherein the sample holder comprises a first plate, and a second plate, and spacers.

The apparatus, kit, or method of any prior embodiments, wherein the sample holder comprises a first plate, a second plate, and spacers, wherein the spacers are configured to regulate a gap between the plates when the plates are pressed against each, compressing the sample into a thin layer.

The apparatus, kit, or method of any prior embodiments, wherein the sample holder comprises a first plate, a second plate, and spacers, and wherein:

i. the plates are moveable relative to each other into different configurations, including an open configuration and a closed configuration;

ii. in the open configuration: the two plates are separated apart, the spacing between the plates is not regulated by the spacers, and the sample is deposited on one or both of the plates; and

iii. in the closed configuration, which is configured after the sample deposition in the open configuration: at least part of the sample is compressed by the two plates into a layer of highly uniform thickness and is substantially stagnant relative to the plates, wherein the uniform thickness of the layer is regulated by the plates and the spacers.

The apparatus, kit, or method of any prior embodiments, wherein the sample holder comprises a Q-card, which comprises a first plate, a second plate, and spacers, wherein the spacers are configured to regulate a gap between the plates when the plates are pressed against each, compressing the sample into a thin layer.

The apparatus, kit, or method of any prior embodiments, wherein

i. the sample holder comprises a first plate, a second plate, and spacers, wherein the spacers have a uniform height and a constant inter-spacer distance; and

ii. the sample is compressed by the sample holder into a thin layer with a uniform thickness that is regulated by the height of the spacers.

The apparatus, kit, or method of any prior embodiments, wherein the sample is compressed into a layer of uniform thickness that substantially equals uniform height of spacers that are fixed to one or both of the plates.

The apparatus, kit or method of any prior embodiments, wherein the sample is compressed into a layer of uniform thickness that has a variation of less than 15%, 10%, 5%, 2%, 1%, or in a range between any of the two values.

The apparatus, kit, or method of any prior embodiments, wherein the sample, when compressed, has a thickness of 500 nm or less, 1000 nm or less, 2 μm (micron) or less, 5 μm or less, 10 μm or less, 20 μm or less, 50 μm or less, 100 μm or less, 150 μm or less, 200 μm or less, 300 μm or less, 500 μm or less, 800 μm or less, 1 mm (millimeter) or less, 2 mm or less, 3 mm or less, 5 mm or less, 10 mm or less, or in a range between any two of these values.

The apparatus, kit, or method of any prior embodiments, wherein the sample holder comprises a first plate and a second plate, wherein each of the plate has a thickness of 500 nm or less, 1000 nm or less, 2 μm (micron) or less, 5 μm or less, 10 μm or less, 20 μm or less, 50 μm or less, 100 μm or less, 150 μm or less, 200 μm or less, 300 μm or less, 500 μm or less, 800 μm or less, 1 mm (millimeter) or less, 2 mm or less, 3 mm or less, 5 mm or less, 10 mm or less, or in a range between any two of these values.

### Imager

The apparatus, kit, or method of any prior embodiments, wherein the imager comprises a camera.

The apparatus, kit, or method of any prior embodiments, wherein the imager is a part of the detector.

The apparatus, kit, or method of any prior embodiments, wherein the imager is the entirety of the detector.

The apparatus, kit, or method of any prior embodiments, wherein the imager is directed by the software to capture one or more images of the sample, identify the interference element regions and the interference element free regions, and digitally separate the interference element regions from the interference element free regions.

The apparatus, kit, or method of any prior embodiments, wherein the imager comprises a filter that is configured to filter signals from the sample.

The apparatus, kit, or method of any prior embodiments, wherein the imager comprises a light source that is configured to illuminate the sample.

### Detector:

The apparatus, kit, or method of any prior embodiments, wherein the detector is a mobile device.

The apparatus, kit, or method of any prior embodiments, wherein the detector is a smart phone.

The apparatus, kit, or method of any prior embodiments, wherein the detector is a smart phone and the imager is a camera as part of the smart phone.

The apparatus, kit, or method of any prior embodiments, wherein the detector comprises a display that is configured to show the presence and/or amount of the analyte.

The apparatus, kit, or method of any prior embodiments, wherein the detector is configured to transmit detection results to a third party.

### Software

The apparatus, kit, or method of any prior embodiments, wherein the software is stored in a storage unit, which is part of the detector.

The apparatus, kit, or method of any prior embodiments, wherein the software is configured to direct the detector to display the presence and/or amount of the analyte.

The apparatus, kit, or method of any prior embodiments, wherein the software is configured to direct the imager to calculate the combined signal of the analyte from the interference element free regions.

The apparatus, kit, or method of any prior embodiments, wherein the software is configured to direct the imager to disregard the signal of the analyte from the interference element regions.

The apparatus, kit, or method of any prior embodiments, wherein the software is configured to direct the imager to increase signal contrast of the signals from the interference element regions to the signals from the interference element free regions

The apparatus, kit, or method of any prior embodiments, wherein the software is configured to direct the detector to calculate a ratio of the signal from the interference element regions to the interference element free regions.

### Mobile Apparatus

The device, method, or system of any prior embodiments, wherein the mobile apparatus is a smart phone.

The device, method, or system of any prior embodiments, wherein the mobile apparatus comprises a set of instructions that, when executed, direct the apparatus to capture one or more images of the sample,

The device, method, or system of any prior embodiments, wherein the mobile apparatus comprises a light source that is configured to illuminate the sample.

The device, method, or system of any prior embodiments, wherein the mobile apparatus comprises a display that is configured to show the presence and/or amount of the analyte.

The device, method, or system of any prior embodiments, wherein the mobile apparatus comprises a set of instructions that, when executed, direct the detector to display the presence and/or amount of the analyte.

The device, method, or system of any prior embodiments, wherein the mobile apparatus is configured to transmit detection results to a third party.

### Adaptor

The device, method, or system of any prior embodiments, wherein the adaptor comprises a filter that is configured to filter signals from the sample.

The device, method, or system of any prior embodiments, wherein the adaptor comprises a card slot, into which the device can be inserted.

The device, method, or system of any prior embodiments, wherein the adaptor comprises a slider that facilitates the insertion of the device into the card slot.

The device, method, or system of any prior embodiments, wherein the adaptor comprises a holder frame that is configured to removably connect to the mobile apparatus.

The device, method, or system of any prior embodiments, wherein the adaptor comprises an optical box that includes one or more optical components that are configured to enhance the signal from the sample.

### Fields and Applications:

The apparatus, kit, or method of any prior embodiments, wherein the apparatus or method are used for detection of proteins, peptides, nucleic acids, synthetic compounds, inorganic compounds, organic compounds, bacteria, virus, cells, tissues, nanoparticles, and other molecules, compounds, mixtures and substances thereof.

The apparatus, kit, or method of any prior embodiments, wherein the apparatus or method are used for diagnostics, management, and/or prevention of human diseases and conditions.

The apparatus, kit, or method of any prior embodiments, wherein the apparatus or method are used for diagnostics, management, and/or prevention of veterinary diseases and conditions, or for diagnostics, management, and/or prevention of plant diseases and conditions.

The apparatus, kit, or method of any prior embodiments, wherein the apparatus or method are used for environments testing and decontamination.

The apparatus, kit, or method of any prior embodiments, wherein the apparatus or method are used for agricultural or veterinary applications.

The apparatus, kit, or method of any prior embodiments, wherein the apparatus or method are used for food testing.

The apparatus, kit, or method of any prior embodiments, wherein the apparatus or method are used for drug testing and prevention.

The apparatus, kit, or method of any prior embodiments, wherein the apparatus or method are used for detecting and/or measuring an analyte in blood.

The apparatus, kit, or method of any prior embodiments, wherein the apparatus or method are used for a colorimetric assay.

The apparatus, kit, or method of any prior embodiments, wherein the apparatus or method are used for a fluorescence assay.

### Signal Related to Analyte

The apparatus, kit, or method of any prior embodiments, wherein the signal related to the analyte is an electrical signal or an optical signal.

The apparatus, kit, or method of any prior embodiments, wherein the signal related to the analyte is an optical signal that allows the imager to capture images of the interference element rich region and the interference element poor region. The apparatus, kit, or method of any prior embodiments, wherein the signal related to the analyte is from a colorimetric reaction. The apparatus, kit, or method of any prior embodiments, wherein the signal related to the analyte is produced by illuminating the sample with an illumination source.

### Spacers and Plates

The apparatus, kit, or method of any prior embodiments, wherein the plates are movable relative to each.

The apparatus, kit, or method of any prior embodiments, wherein the spacers are fixed on one or both of the plates and have a uniform height.

The apparatus, kit, or method of any prior embodiments, wherein the first plate and second plate are configured to compress the sample into a layer of uniform thickness that substantially equals the height of the spacers.

The apparatus, kit, or method of any prior embodiments, wherein the spacers have a uniform height of 1 mm or less, 500 μm or less, 400 μm or less, 300 μm or less, 200 μm or less, 175 μm or less, 150 μm or less, 125 μm or less, 100 μm or less, 75 μm or less, 50 μm or less, 40 μm or less, 30 μm or less, 20 μm or less, 10 μm or less, 5 μm or less, 4 μm or less, 3 μm or less, 2 μm or less, 1.8 μm or less, 1.5 μm or less, 1 μm or less, 0.5 μm or less, 0.2 μm or less, 0.1 μm or less, 50 nm or less, 20 nm or less, 10 nm or less, or in a range between any of the two values.

The apparatus, kit, or method of any prior embodiments, wherein the spacers have a uniform height in the range of 0.5-2 μm, 0.5-3 μm, 0.5-5 μm, 0.5-10 μm, 0.5-20 μm, 0.5-30 μm, or 0.5-50 μm.

The apparatus, kit, or method of any prior embodiments, wherein at least one of the plates has a thickness of 100 mm or less, 50 mm or less, 25 mm or less, 10 mm or less, 5 mm or less, 1 mm or less, 500 μm or less, 400 μm or less, 300 μm or less, 200 μm or less, 175 μm or less, 150 μm or less, 125 μm or less, 100 μm or less, 75 μm or less, 50 μm or less, 40 μm or less, 30 μm or less, 20 μm or less, 10 μm or less, 5 μm or less, 4 μm or less, 3 μm or less, 2 μm or less, 1.8 μm or less, 1.5 μm or less, 1 μm or less, 0.5 μm or less, 0.2 μm or less, or 0.1 μm or less, or in a range between any of the two values.

The apparatus, kit, or method of any prior embodiments, wherein at least one of the plates has a thickness in the range of 0.5 to 1.5 mm; around 1 mm; in the range of 0.15 to 0.2 mm; or around 0.175 mm.

The apparatus, kit, or method of any prior embodiments, wherein at least one of the plates has a lateral area of 1 mm² or less, 10 mm² or less, 25 mm² or less, 50 mm² or less, 75 mm² or less, 1 cm² (square centimeter) or less, 2 cm² or less, 3 cm² or less, 4 cm² or less, 5 cm² or less, 10 cm² or less, 100 cm² or less, 500 cm² or less, 1,000 cm² or less, 5,000 cm² or less, 10,000 cm² or less, 10,000 cm² or less, or in a range between any two of these values

The apparatus, kit, or method of any prior embodiments, wherein at least one of the plates has a lateral area of in the range of 500 to 1000 mm²; or around 750 mm²

The apparatus, kit, or method of any prior embodiments, wherein the Young's modulus of the spacers times the filling factor of the spacers is equal or larger than 10 MPa, wherein the filling factor is the ratio of the spacer area in contact with the layer of uniform thickness to the total plate area in contact with the layer of uniform thickness.

The apparatus, kit, or method of any prior embodiments, wherein the thickness of the flexible plate times the Young's modulus of the flexible plate is in the range 60 to 750 GPa-μm.

The apparatus, kit, or method of any prior embodiments, wherein for a flexible plate, the fourth power of the inter-spacer-distance (ISD) divided by the thickness of the flexible plate (h) and the Young's modulus (E) of the flexible plate, ISD⁴/(hE), is equal to or less than 10⁶ μm³/GPa.

The apparatus, kit, or method of any prior embodiments, wherein one or both plates comprises a location marker, either on a surface of or inside the plate, that provide information of a location of the plate.

The apparatus, kit, or method of any prior embodiments, wherein one or both plates comprises a scale marker, either on a surface of or inside the plate, that provide information of a lateral dimension of a structure of the sample and/or the plate.

The apparatus, kit, or method of any prior embodiments, wherein one or both plates comprises an image marker, either on a surface of or inside the plate, that assists an imaging of the sample.

The apparatus, kit, or method of any prior embodiments, wherein the inter-spacer distance is in the range of 7 μm to 50 μm.

The apparatus, kit, or method of any prior embodiments, wherein the inter-spacer distance is in the range of 50 μm to 120 μm.

The apparatus, kit, or method of any prior embodiments, wherein the inter-spacer distance is in the range of 120 μm to 200 μm.

The apparatus, kit, or method of any prior embodiments, wherein the spacers are pillars with a cross-sectional shape selected from round, polygonal, circular, square, rectangular, oval, elliptical, or any combination of the same.

The apparatus, kit, or method of any prior embodiments, wherein the spacers have a pillar shape and have a substantially flat top surface, wherein, for each spacer, the ratio of the lateral dimension of the spacer to its height is at least 1.

The apparatus, kit, or method of any prior embodiments, wherein each spacer has the ratio of the lateral dimension of the spacer to its height is at least 1.

The apparatus, kit, or method of any prior embodiments, wherein the minimum lateral dimension of spacer is less than or substantially equal to the minimum dimension of an analyte in the sample.

The apparatus, kit, or method of any prior embodiments, wherein the minimum lateral dimension of spacer is in the range of 0.5 μm to 100 μm.

The apparatus, kit, or method of any prior embodiments, wherein the minimum lateral dimension of spacer is in the range of 0.5 μm to 10 μm.

The apparatus, kit, or method of any prior embodiments, wherein the spacers have a pillar shape, and the sidewall corners of the spacers have a round shape with a radius of curvature at least 1 μm.

The apparatus, kit, or method of any prior embodiments, wherein the spacers have a density of at least 100/mm².

The apparatus, kit, or method of any prior embodiments, wherein the spacers have a density of at least 1,000/mm².

The apparatus, kit, or method of any prior embodiments, wherein at least one of the plates is transparent.

The apparatus, kit, or method of any prior embodiments, wherein at least one of the plates is made from a flexible polymer.

The apparatus, kit, or method of any prior embodiments, wherein, for a pressure that compresses the plates, the spacers are not compressible and/or, independently, only one of the plates is flexible.

The apparatus, kit, or method of any prior embodiments, wherein the flexible plate has a thickness in the range of 10 μm to 200 μm.

The apparatus, kit, or method of any prior embodiments, wherein the variation of sample thickness is less than 30%.

The apparatus, kit, or method of any prior embodiments, wherein the variation of sample thickness is less than 10%.

The apparatus, kit, or method of any prior embodiments, wherein the variation of sample thickness is less than 5%.

The apparatus, kit, or method of any prior embodiments, wherein the first and second plates are connected and are configured to be changed from the open configuration to the closed configuration by folding the plates.

The apparatus, kit, or method of any prior embodiments, wherein the first and second plates are connected by a hinge and are configured to be changed from the open configuration to the closed configuration by folding the plates along the hinge.

The apparatus, kit, or method of any prior embodiments, wherein the first and second plates are connected by a hinge that is a separate material to the plates, and are configured to be changed from the open configuration to the closed configuration by folding the plates along the hinge.

The apparatus, kit, or method of any prior embodiments, wherein the first and second plates are made in a single piece of material and are configured to be changed from the open configuration to the closed configuration by folding the plates.

The apparatus, kit, or method of any prior embodiments, wherein the layer of uniform thickness sample is uniform over a lateral area that is at least 1 mm².

The apparatus, kit, or method of any prior embodiments, wherein the spacers are fixed on a plate by directly embossing the plate or injection molding of the plate.

The apparatus, kit, or method of any prior embodiments, wherein the materials of the plate and the spacers are selected from polystyrene, PMMA, PC, COC, COP, or another plastic.

Device and Assay with High Uniformity

### Flat Top of Pillar Spacers

In certain embodiments of the present invention, the spacers are pillars that have a flat top and a foot fixed on one plate, wherein the flat top has a smoothness with a small surface variation, and the variation is less than 5, 10 nm, 20 nm, 30 nm, 50 nm, 100 nm, 200 nm, 300 nm, 400 nm, 500 nm, 600 nm, 700 nm, 800 nm, 1000 nm, or in a range between any two of the values. A preferred flat pillar top smoothness is that surface variation of 50 nm or less.

Furthermore, the surface variation is relative to the spacer height and the ratio of the pillar flat top surface variation to the spacer height is less than 0.5%, 1%, 3%, 5%, 7%, 10%, 15%, 20%, 30%, 40%, or in a range between any two of the values. A preferred flat pillar top smoothness has a ratio of the pillar flat top surface variation to the spacer height is less than 2%, 5%, or 10%.

### Sidewall Angle of Pillar Spacers

In certain embodiments of the present invention, the spacers are pillars that have a sidewall angle. In some embodiments, the sidewall angle is less than 5 degree (measured from the normal of a surface), 10 degree, 20 degree, 30 degree, 40 degree, 50 degree, 70 degree, or in a range between any two of the values. In a preferred embodiment, the sidewall angle is less 5 degree, 10 degree, or 20 degree.

### Formation of Uniform Thin Fluidic Layer by an Imprecise Force Pressing

In certain embodiment of the present invention, a uniform thin fluidic sample layer is formed by using a pressing with an imprecise force. The term “imprecise pressing force” without adding the details and then adding a definition for imprecise pressing force. As used herein, the term “imprecise” in the context of a force (e.g. “imprecise pressing force”) refers to a force that

(a) has a magnitude that is not precisely known or precisely predictable at the time the force is applied; (b) has a pressure in the range of 0.01 kg/cm² (centimeter square) to 100 kg/cm², (c) varies in magnitude from one application of the force to the next; and (d) the imprecision (i.e. the variation) of the force in (a) and (c) is at least 20% of the total force that actually is applied.

An imprecise force can be applied by human hand, for example, e.g., by pinching an object together between a thumb and index finger, or by pinching and rubbing an object together between a thumb and index finger.

In some embodiments, the imprecise force by the hand pressing has a pressure of 0.01 kg/cm2, 0.1 kg/cm2, 0.5 kg/cm2, 1 kg/cm2, 2 kg/cm2, kg/cm2, 5 kg/cm2, 10 kg/cm2, 20 kg/cm2, 30 kg/cm2, 40 kg/cm2, 50 kg/cm2, 60 kg/cm2, 100 kg/cm2, 150 kg/cm2, 200 kg/cm2, or a range between any two of the values; and a preferred range of 0.1 kg/cm2 to 0.5 kg/cm2, 0.5 kg/cm2 to 1 kg/cm2, 1 kg/cm2 to 5 kg/cm2, 5 kg/cm2 to 10 kg/cm2 (Pressure).

### Spacer Filling Factor.

The term “spacer filling factor” or “filling factor” refers to the ratio of the spacer contact area to the total plate area”, wherein the spacer contact area refers, at a closed configuration, the contact area that the spacer's top surface contacts to the inner surface of a plate, and the total plate area refers the total area of the inner surface of the plate that the flat top of the spacers contact. Since there are two plates and each spacer has two contact surfaces each contacting one plate, the filling fact is the filling factor of the smallest.

For example, if the spacers are pillars with a flat top of a square shape (10 μm×10 μm), a nearly uniform cross-section and 2 μm tall, and the spacers are periodic with a period of 100 μm, then the filing factor of the spacer is 1%. If in the above example, the foot of the pillar spacer is a square shape of 15 μm×15 μm, then the filling factor is still 1% by the definition.

### EXAMPLES OF PRESENT INVENTION

In certain embodiments, a device for forming a thin fluidic sample layer with a uniform predetermined thickness by pressing, comprising:

a first plate, a second plate, and spacers, wherein:
i. the plates are movable relative to each other into different configurations;
ii. one or both plates are flexible;
iii. each of the plates comprises an inner surface that has a sample contact area for contacting a fluidic sample;
iv. each of the plates comprises, on its respective outer surface, a force area for applying an pressing force that forces the plates together;
v. one or both of the plates comprise the spacers that are permanently fixed on the inner surface of a respective plate;
vi. the spacers have a predetermined substantially uniform height that is equal to or less than 200 microns, and a predetermined fixed inter-spacer-distance;
vii. the fourth power of the inter-spacer-distance (ISD) divided by the thickness (h) and the Young's modulus (E) of the flexible plate (ISD⁴/(hE)) is 5×10⁶ μm³/GPa or less; and
viii. at least one of the spacers is inside the sample contact area;

wherein one of the configurations is an open configuration, in which: the two plates are partially or completely separated apart, the spacing between the plates is not regulated by the spacers, and the sample is deposited on one or both of the plates;

wherein another of the configurations is a closed configuration which is configured after the sample is deposited in the open configuration and the plates are forced to the closed configuration by applying the pressing force on the force area; and in the closed configuration: at least part of the sample is compressed by the two plates into a layer of highly uniform thickness and is substantially stagnant relative to the plates, wherein the uniform thickness of the layer is confined by the sample contact areas of the two plates and is regulated by the plates and the spacers.

In certain embodiments, a method of forming a thin fluidic sample layer with a uniform predetermined thickness by pressing, comprising the steps of:
(a) obtaining a device of embodiment AA1;
(b) depositing a fluidic sample on one or both of the plates; when the plates are configured in an open configuration, wherein the open configuration is a configuration in which the two plates are partially or completely separated apart and the spacing between the plates is not regulated by the spacers;
(c) after (b), forcing the two plates into a closed configuration, in which: at least part of the sample is compressed by the two plates into a layer of substantially uniform thickness, wherein the uniform thickness of the layer is confined by the sample contact surfaces of the plates and is regulated by the plates and the spacers.

In certain embodiments, a device for analyzing a fluidic sample, comprising:

a first plate, a second plate, and spacers, wherein:
i. the plates are movable relative to each other into different configurations;
ii. one or both plates are flexible;
iii. each of the plates has, on its respective inner surface, a sample contact area for contacting a fluidic sample,
iv. one or both of the plates comprise the spacers and the spacers are fixed on the inner surface of a respective plate;
v. the spacers have a predetermined substantially uniform height that is equal to or less than 200 microns, and the inter-spacer-distance is predetermined;
vi. the Young's modulus of the spacers multiplied by the filling factor of the spacers is at least 2 MPa; and
vii. at least one of the spacers is inside the sample contact area; and

wherein one of the configurations is an open configuration, in which: the two plates are partially or completely separated apart, the spacing between the plates is not regulated by the spacers, and the sample is deposited on one or both of the plates; and

wherein another of the configurations is a closed configuration which is configured after the sample is deposited in the open configuration; and in the closed configuration: at least part of the sample is compressed by the two plates into a layer of highly uniform thickness, wherein the uniform thickness of the layer is confined by the sample contact surfaces of the plates and is regulated by the plates and the spacers.

In certain embodiments, a method of forming a thin fluidic sample layer with a uniform predetermined thickness by pressing, comprising the steps of:
(a) obtaining a device of embodiment AA3;
(b) depositing a fluidic sample on one or both of the plates; when the plates are configured in an open configuration, wherein the open configuration is a configuration in which the two plates are partially or completely separated apart and the spacing between the plates is not regulated by the spacers;
(c) after (b), forcing the two plates into a closed configuration, in which: at least part of the sample is compressed by the two plates into a layer of substantially uniform thickness, wherein the uniform thickness of the layer is confined by the sample contact surfaces of the plates and is regulated by the plates and the spacers.

In certain embodiments, a device for analyzing a fluidic sample, comprising:

a first plate and a second plate, wherein:
i. the plates are movable relative to each other into different configurations;
ii. one or both plates are flexible;
iii. each of the plates has, on its respective surface, a sample contact area for contacting a sample that contains an analyte,
iv. one or both of the plates comprise spacers that are permanently fixed to a plate within a sample contact area, wherein the spacers have a predetermined substantially uniform height and a predetermined fixed inter-spacer distance that is at least about 2 times larger than the size of the analyte, up to 200 μm, and wherein at least one of the spacers is inside the sample contact area;

wherein one of the configurations is an open configuration, in which: the two plates are separated apart, the spacing between the plates is not regulated by the spacers, and the sample is deposited on one or both of the plates; and

wherein another of the configurations is a closed configuration which is configured after the sample deposition in the open configuration; and in the closed configuration: at least part of the sample is compressed by the two plates into a layer of highly uniform thickness, wherein the uniform thickness of the layer is confined by the sample contact surfaces of the plates and is regulated by the plates and the spacers.

In certain embodiments, a method of forming a thin fluidic sample layer with a uniform predetermined thickness by pressing, comprising the steps of:
(a) obtaining a device of embodiment AA5;
(b) depositing a fluidic sample on one or both of the plates; when the plates are configured in an open configuration, wherein the open configuration is a configuration in which the two plates are partially or completely separated apart and the spacing between the plates is not regulated by the spacers;
(c) after (b), forcing the two plates into a closed configuration, in which: at least part of the sample is compressed by the two plates into a layer of substantially uniform thickness, wherein the uniform thickness of the layer is confined by the sample contact surfaces of the plates and is regulated by the plates and the spacers.

In certain embodiments, a device for forming a thin fluidic sample layer with a uniform predetermined thickness by pressing, comprising:

a first plate, a second plate, and spacers, wherein:
i. the plates are movable relative to each other into different configurations;
ii. one or both plates are flexible;
iii. each of the plates comprises, on its respective inner surface, a sample contact area for contacting and/or compressing a fluidic sample;
iv. each of the plates comprises, on its respective outer surface, an area for applying a force that forces the plates together;
v. one or both of the plates comprise the spacers that are permanently fixed on the inner surface of a respective plate;
vi. the spacers have a predetermined substantially uniform height that is equal to or less than 200 microns, a predetermined width, and a predetermined fixed inter-spacer-distance;
vii. a ratio of the inter-spacer-distance to the spacer width is 1.5 or larger; and
viii. at least one of the spacers is inside the sample contact area;

wherein one of the configurations is an open configuration, in which: the two plates are partially or completely separated apart, the spacing between the plates is not regulated by the spacers, and the sample is deposited on one or both of the plates;

wherein another of the configurations is a closed configuration which is configured after the sample deposition in the open configuration; and in the closed configuration: at least part of the sample is compressed by the two plates into a layer of highly uniform thickness and is substantially stagnant relative to the plates, wherein the uniform thickness of the layer is confined by the sample contact areas of the two plates and is regulated by the plates and the spacers.

In certain embodiments, a method of forming a thin fluidic sample layer with a uniform predetermined thickness by pressing with an imprecise pressing force, comprising the steps of:
(a) obtaining a device of embodiment AA7;
(b) obtaining a fluidic sample;
(c) depositing the sample on one or both of the plates; when the plates are configured in an open configuration, wherein the open configuration is a configuration in which the two plates are partially or completely separated apart and the spacing between the plates is not regulated by the spacers;
(d) after (c), forcing the two plates into a closed configuration, in which: at least part of the sample is compressed by the two plates into a layer of substantially uniform thickness, wherein the uniform thickness of the layer is confined by the sample contact surfaces of the plates and is regulated by the plates and the spacers.

The devices or methods of any prior embodiment, wherein the spacers have a shape of pillar with a foot fixed on one of the plates and a flat top surface for contacting the other plate.

The devices or methods of any prior embodiment, wherein the spacers have a shape of pillar with a foot fixed on one of the plates, a flat top surface for contacting the other plate, substantially uniform cross-section.

The devices or methods of any prior embodiment, wherein the spacers have a shape of pillar with a foot fixed on one of the plates and a flat top surface for contacting the other plate, wherein the flat top surface of the pillars has a variation in less than 10 nm.

The devices or methods of any prior embodiment, wherein the spacers have a shape of pillar with a foot fixed on one of the plates and a flat top surface for contacting the other plate, wherein the flat top surface of the pillars has a variation in less than 50 nm.

The devices or methods of any prior embodiment, wherein the spacers have a shape of pillar with a foot fixed on one of the plates and a flat top surface for contacting the other plate, wherein the flat top surface of the pillars has a variation in less than 50 nm.

The devices or methods of any prior embodiment, wherein the spacers have a shape of pillar with a foot fixed on one of the plates and a flat top surface for contacting the other plate, wherein the flat top surface of the pillars has a variation in less than 10 nm, 20 nm, 30 nm, 100 nm, 200 nm, or in a range of any two of the values.

The devices or methods of any prior embodiment, wherein the Young's modulus of the spacers multiplied by the filling factor of the spacers is at least 2 MPa.

The devices or methods of any prior embodiment, wherein the sample comprises an analyte and the predetermined constant inter-spacer distance is at least about 2 times larger than the size of the analyte, up to 200 μm.

The devices or methods of any prior embodiment, wherein the sample comprise an analyte, the predetermined constant inter-spacer distance is at least about 2 times larger than the size of the analyte, up to 200 μm, and the Young's modulus of the spacers multiplied by the filling factor of the spacers is at least 2 MPa.

The devices or methods of any prior embodiment, wherein a fourth power of the inter-spacer-distance (IDS) divided by the thickness (h) and the Young's modulus (E) of the flexible plate (ISD⁴/(hE)) is 5×10⁶ μm³/GPa or less.

The devices or methods of any prior embodiment, wherein a fourth power of the inter-spacer-distance (IDS) divided by the thickness (h) and the Young's modulus (E) of the flexible plate (ISD⁴/(hE)) is 1×10⁶ μm³/GPa or less.

The devices or methods of any prior embodiment, wherein a fourth power of the inter-spacer-distance (IDS) divided by the thickness (h) and the Young's modulus (E) of the flexible plate (ISD⁴/(hE)) is 5×10⁵ μm³/GPa or less.

The devices or methods of any prior embodiment, wherein the Young's modulus of the spacers multiplied by the filling factor of the spacers is at least 2 MPa, and a fourth power of the inter-spacer-distance (IDS) divided by the thickness (h) and the Young's modulus (E) of the flexible plate (ISD⁴/(hE)) is 1×10⁵ μm³/GPa or less.

The devices or methods of any prior embodiment, wherein the Young's modulus of the spacers multiplied by the filling factor of the spacers is at least 2 MPa, and a fourth power of the inter-spacer-distance (IDS) divided by the thickness (h) and the Young's modulus (E) of the flexible plate (ISD⁴/(hE)) is 1×10⁴ um³/GPa or less.

The devices or methods of any prior embodiment, wherein the Young's modulus of the spacers multiplied by the filling factor of the spacers is at least 20 MPa.

The devices or methods of any prior embodiment, wherein the ratio of the inter-spacing distance of the spacers to the average width of the spacer is 2 or larger.

The devices or methods of any prior embodiment, wherein the ratio of the inter-spacing distance of the spacers to the average width of the spacer is 2 or larger, and the Young's modulus of the spacers multiplied by the filling factor of the spacers is at least 2 MPa.

The devices or methods of any prior embodiment, wherein inter-spacer distance that is at least about 2 times larger than the size of the analyte, up to 200 μm.

The devices or methods of any prior embodiment, wherein a ratio of the inter-spacer-distance to the spacer width is 1.5 or larger.

The devices or methods of any prior embodiment, wherein a ratio of the width to the height of the spacer is 1 or larger.

The devices or methods of any prior embodiment, wherein a ratio of the width to the height of the spacer is 1.5 or larger.

The devices or methods of any prior embodiment, wherein a ratio of the width to the height of the spacer is 2 or larger.

The devices or methods of any prior embodiment, wherein a ratio of the width to the height of the spacer is larger than 2, 3, 5, 10, 20, 30, 50, or in a range of any two the value.

The methods of any prior embodiment, wherein the force that presses the two plates into the closed configuration is an imprecise pressing force.

The methods of any prior embodiment, wherein the force that presses the two plates into the closed configuration is an imprecise pressing force provided by human hand.

The methods of any prior embodiment, wherein the forcing of the two plates to compress at least part of the sample into a layer of substantially uniform thickness comprises a use of a conformable pressing, either in parallel or sequentially, an area of at least one of the plates to press the plates together to a closed configuration, wherein the conformable pressing generates a substantially uniform pressure on the plates over the at least part of the sample, and the pressing spreads the at least part of the sample laterally between the sample contact surfaces of the plates, and wherein the closed configuration is a configuration in which the spacing between the plates in the layer of uniform thickness region is regulated by the spacers; and wherein the reduced thickness of the sample reduces the time for mixing the reagents on the storage site with the sample.

The methods of any prior embodiment, wherein the pressing force is an imprecise force that has a magnitude which is, at the time that the force is applied, either (a) unknown and unpredictable, or (b) cannot be known and cannot be predicted within an accuracy equal or better than 20% of the average pressing force applied.

The methods of any prior embodiment, wherein the pressing force is an imprecise force that has a magnitude which is, at the time that the force is applied, either (a) unknown and unpredictable, or (b) cannot be known and cannot be predicted within an accuracy equal or better than 30% of the average pressing force applied.

The methods of any prior embodiment, wherein the pressing force is an imprecise force that has a magnitude which is, at the time that the force is applied, either (a) unknown and unpredictable, or (b) cannot be known and cannot be predicted within an accuracy equal or better than 30% of the average pressing force applied; and wherein the layer of highly uniform thickness has a variation in thickness uniform of 20% or less.

The methods of any prior embodiment, wherein the pressing force is an imprecise force that has a magnitude which cannot, at the time that the force is applied, be determined within an accuracy equal or better than 30%, 40%, 50%, 70%, 100%, 200%, 300%, 500%, 1,000%, 2,000%, or in a range between any of the two values.

The devices or methods of any prior embodiment, wherein the flexible plate has a thickness of in the range of 10 μm to 200 μm.

The devices or methods of any prior embodiment, wherein the flexible plate has a thickness of in the range of 20 μm to 100 μm.

The devices or methods of any prior embodiment, wherein the flexible plate has a thickness of in the range of 25 μm to 180 μm.

The devices or methods of any prior embodiment, wherein the flexible plate has a thickness of in the range of 200 μm to 260 μm.

The devices or methods of any prior embodiment, wherein the flexible plate has a thickness of equal to or less than 250 μm, 225 μm, 200 μm, 175 μm, 150 μm, 125 μm, 100 μm, 75 μm, 50 μm, 25 μm, 10 μm, 5 μm, 1 μm, or in a range between the two of the values.

The devices or methods of any prior method, wherein the sample has a viscosity in the range of 0.1 to 4 (mPa s).

The devices or methods of any prior embodiment, wherein the flexible plate has a thickness of in the range of 200 μm to 260 μm.

The devices or methods of any prior embodiment, wherein the flexible plate has a thickness in the range of 20 μm to 200 μm and Young's modulus in the range 0.1 to 5 GPa.

The method of any prior claim, wherein the sample deposition of step (b) is a deposition directly from a subject to the plate without using any transferring devices.

The method any prior claim, wherein during the deposition of step (b), the amount of the sample deposited on the plate is unknown.

The method of any prior claim, wherein the method further comprises a analyzing step (e) that analyze the sample.

The method of any prior claim, wherein the analyzing step (e) comprises calculating the volume of a relevant sample volume by measuring the lateral area of the relevant sample volume and calculating the volume from the lateral area and the predetermined spacer height.

The method of any prior claim, wherein the analyzing step (e) comprises measuring:
i. imaging, luminescence selected from photoluminescence, electroluminescence, and electrochemiluminescence,
iii. surface Raman scattering,
iv. electrical impedance selected from resistance, capacitance, and inductance, or
v. any combination of i-iv.

The method of any prior claim, wherein the analyzing step (e) comprises reading, image analysis, or counting of the analyte, or a combination of thereof.

The method of any prior claim, wherein the sample contains one or plurality of analytes, and one or both plate sample contact surfaces comprise one or a plurality of binding sites that each binds and immobilize a respective analyte.

The method of any prior claim, wherein one or both plate sample contact surfaces comprise one or a plurality of storage sites that each stores a reagent or reagents, wherein the reagent(s) dissolve and diffuse in the sample during or after step (c).

The method of any prior claim, wherein one or both plate sample contact surfaces comprises one or a plurality of amplification sites that are each capable of amplifying a signal from the analyte or a label of the analyte when the analyte or label is within 500 nm from an amplification site.

The method of any prior claim, wherein:

i. one or both plate sample contact surfaces comprise one or a plurality of binding sites that each binds and immobilize a respective analyte; or

ii. one or both plate sample contact surfaces comprise, one or a plurality of storage sites that each stores a reagent or reagents; wherein the reagent(s) dissolve and diffuse in the sample during or after step (c), and wherein the sample contains one or plurality of analytes; or

iii. one or a plurality of amplification sites that are each capable of amplifying a signal from the analyte or a label of the analyte when the analyte or label is 500 nm from the amplification site; or

iv. any combination of i to iii.

The devices or methods of any prior embodiment, wherein the liquid sample is a biological sample selected from amniotic fluid, aqueous humour, vitreous humour, blood (e.g., whole blood, fractionated blood, plasma or serum), breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensates, sebum, semen, sputum, sweat, synovial fluid, tears, vomit, and urine.

The devices or methods of any prior embodiment, wherein the layer of uniform thickness in the closed configuration is less than 150 μm.

The method of any prior embodiment, wherein the pressing is provided by a pressured liquid, a pressed gas, or a conformal material.

The method of any prior claim, wherein the analyzing comprises counting cells in the layer of uniform thickness.

The method of any prior embodiment, wherein the analyzing comprises performing an assay in the layer of uniform thickness.

The devices or methods of any prior embodiment, wherein the assay is a binding assay or biochemical assay.

The method of any prior claim, wherein the sample deposited has a total volume less 0.5 μL.

The method of any prior claim, wherein multiple drops of sample are deposited onto one or both of the plates.

The devices or methods of any prior embodiment, wherein the inter-spacer distance is in the range of 1 μm to 120 μm.

The devices or methods of any prior embodiment, wherein the inter-spacer distance is in the range of 120 μm to 50 μm.

The devices or methods of any prior embodiment, wherein the inter-spacer distance is in the range of 120 μm to 200 μm.

The device of any prior device claim, wherein the flexible plates have a thickness in the range of 20 μm to 250 μm and Young's modulus in the range 0.1 to 5 GPa.

The device of any prior device claim, wherein for a flexible plate, the thickness of the flexible plate times the Young's modulus of the flexible plate is in the range 60 to 750 GPa-μm.

The device of any prior device claim, wherein the layer of uniform thickness sample is uniform over a lateral area that is at least 1 mm².

The device of any prior device claim, wherein the layer of uniform thickness sample is uniform over a lateral area that is at least 3 mm².

The device of any prior device claim, wherein the layer of uniform thickness sample is uniform over a lateral area that is at least 5 mm².

The device of any prior device claim, wherein the layer of uniform thickness sample is uniform over a lateral area that is at least 10 mm².

The device of any prior device claim, wherein the layer of uniform thickness sample is uniform over a lateral area that is at least 20 mm².

The device of any prior device claim, wherein the layer of uniform thickness sample is uniform over a lateral area that is in a range of 20 mm² to 100 mm².

The device of any prior device claim, wherein the layer of uniform thickness sample has a thickness uniformity of up to +/−5% or better.

The device of any prior device claim, wherein the layer of uniform thickness sample has a thickness uniformity of up to +/−10% or better.

The device of any prior device claim, wherein the layer of uniform thickness sample has a thickness uniformity of up to +/−20% or better.

The device of any prior device claim, wherein the layer of uniform thickness sample has a thickness uniformity of up to +/−30% or better.

The device of any prior device claim, wherein the layer of uniform thickness sample has a thickness uniformity of up to +/−40% or better.

The device of any prior device claim, wherein the layer of uniform thickness sample has a thickness uniformity of up to +/−50% or better.

The device of any prior device claim, wherein the spacers are pillars with a cross-sectional shape selected from round, polygonal, circular, square, rectangular, oval, elliptical, or any combination of the same.

The device of any prior device claim, wherein the spacers have pillar shape, have a substantially flat top surface, and have substantially uniform cross-section, wherein, for each spacer, the ratio of the lateral dimension of the spacer to its height is at least 1.

The device of any prior device claim, wherein the inter spacer distance is periodic.

The device of any prior device claim, wherein the spacers have a filling factor of 1% or higher, wherein the filling factor is the ratio of the spacer contact area to the total plate area.

The device of any prior device claim, wherein the Young's modulus of the spacers times the filling factor of the spacers is equal or larger than 20 MPa, wherein the filling factor is the ratio of the spacer contact area to the total plate area.

The device of any prior device claim, wherein the spacing between the two plates at the closed configuration is in less 200 μm.

The device of any prior device claim, wherein the spacing between the two plates at the closed configuration is a value selected from between 1.8 μm and 3.5 μm.

The device of any prior device claim, wherein the spacing are fixed on a plate by directly embossing the plate or injection molding of the plate.

The device of any prior device claim, wherein the materials of the plate and the spacers are selected from polystyrene, PMMA, PC, COC, COP, or another plastic.

The device of any prior device claim, wherein the spacers have a pillar shape, and the sidewall corners of the spacers have a round shape with a radius of curvature at least 1 μm.

The device of any prior device claim, wherein the spacers have a density of at least 1,000/mm².

The device of any prior device claim, wherein at least one of the plates is transparent.

The device of any prior device claim, wherein the mold used to make the spacers is fabricated by a mold containing features that are fabricated by either (a) directly reactive ion etching or ion beam etched or (b) by a duplication or multiple duplication of the features that are reactive ion etched or ion beam etched.

The devices or methods of any prior embodiment, wherein the spacers are configured, such that the filling factor is in the range of 1% to 5%.

The devices or methods of any prior embodiment, wherein the surface variation is relative to the spacer height and the ratio of the pillar flat top surface variation to the spacer height is less than 0.5%, 1%, 3%, 5%, 7%, 10%, 15%, 20%, 30%, 40%, or in a range between any two of the values. A preferred flat pillar top smoothness has a ratio of the pillar flat top surface variation to the spacer height is less than 2%, 5%, or 10%.

The devices or methods of any prior embodiment, wherein the spacers are configured, such that the filling factor is in the range of 1% to 5%.

The devices or methods of any prior embodiment, wherein the spacers are configured, such that the filling factor is in the range of 5% to 10%.

The devices or methods of any prior embodiment, wherein the spacers are configured, such that the filling factor is in the range of 10% to 20%.

The devices or methods of any prior embodiment, wherein the spacers are configured, such that the filling factor is in the range of 20% to 30%.

The devices or methods of any prior embodiment, wherein the spacers are configured, such that the filling factor is 5%, 10%, 20%, 30%, 40%, 50%, or in a range of any two of the values.

The devices or methods of any prior embodiment, wherein the spacers are configured, such that the filling factor is 50%, 60%, 70%, 80%, or in a range of any two of the values.

The devices or methods of any prior embodiment, wherein the spacers are configured, such that the filling factor multiplies the Young's modulus of the spacer is in the range of 2 MPa and 10 MPa.

The devices or methods of any prior embodiment, wherein the spacers are configured, such that the filling factor multiplies the Young's modulus of the spacer is in the range of 10 MPa and 20 MPa.

The devices or methods of any prior embodiment, wherein the spacers are configured, such that the filling factor multiplies the Young's modulus of the spacer is in the range of 20 MPa and 40 MPa.

The devices or methods of any prior embodiment, wherein the spacers are configured, such that the filling factor multiplies the Young's modulus of the spacer is in the range of 40 MPa and 80 MPa.

The devices or methods of any prior embodiment, wherein the spacers are configured, such that the filling factor multiplies the Young's modulus of the spacer is in the range of 80 MPa and 120 MPa.

The devices or methods of any prior embodiment, wherein the spacers are configured, such that the filling factor multiplies the Young's modulus of the spacer is in the range of 120 MPa to 150 MPa.

The devices or methods of any prior embodiment, wherein the device further comprises a dry reagent coated on one or both plates.

The devices or methods of any prior embodiment, wherein the device further comprises, on one or both plates, a dry binding site that has a predetermined area, wherein the dry binding site binds to and immobilizes an analyte in the sample.

The devices or methods of any prior embodiment, wherein the device further comprises, on one or both plates, a releasable dry reagent and a release time control material that delays the time that the releasable dry regent is released into the sample.

The device of any prior embodiment, wherein the release time control material delays the time that the dry regent starts is released into the sample by at least 3 seconds.

The device of any prior embodiment, wherein the regent comprises anticoagulant and/or staining reagent(s)

The device of any prior embodiment, wherein the reagent comprises cell lysing reagent(s)

The devices or methods of any prior embodiment, wherein the device further comprises, on one or both plates, one or a plurality of dry binding sites and/or one or a plurality of reagent sites.

The device of any prior device embodiment, wherein the analyte comprises a molecule (e.g., a protein, peptides, DNA, RNA, nucleic acid, or other molecule), cells, tissues, viruses, and nanoparticles with different shapes.

The device of any prior device embodiment, wherein the analyte comprises white blood cells, red blood cells and platelets.

The device of any prior device embodiment, wherein the analyte is stained.

The devices or methods of any prior embodiment, wherein the spacers regulating the layer of uniform thickness have a filling factor of at least 1%, wherein the filling factor is the ratio of the spacer area in contact with the layer of uniform thickness to the total plate area in contact with the layer of uniform thickness.

The devices or methods of any prior embodiment, wherein for spacers regulating the layer of uniform thickness, the Young's modulus of the spacers times the filling factor of the spacers is equal or larger than 10 MPa, wherein the filling factor is the ratio of the spacer area in contact with the layer of uniform thickness to the total plate area in contact with the layer of uniform thickness.

The devices or methods of any prior embodiment, wherein for a flexible plate, the thickness of the flexible plate times the Young's modulus of the flexible plate is in the range 60 to 750 GPa-μm.

The devices or methods of any prior embodiment, wherein for a flexible plate, the fourth power of the inter-spacer-distance (ISD) divided by the thickness of the flexible plate (h) and the Young's modulus (E) of the flexible plate, ISD⁴/(hE), is equal to or less than 10⁶ μm³/GPa,

The devices or methods of any prior embodiment, wherein one or both plates comprises a location marker, either on a surface of or inside the plate, that provide information of a location of the plate.

The devices or methods of any prior embodiment, wherein one or both plates comprises a scale marker, either on a surface of or inside the plate, that provide information of a lateral dimension of a structure of the sample and/or the plate.

The devices or methods of any prior embodiment, wherein one or both plates comprises an imaging marker, either on surface of or inside the plate, that assists an imaging of the sample.

The devices or methods of any prior embodiment, wherein the spacers functions as a location marker, a scale marker, an imaging marker, or any combination of thereof.

The devices or methods of any prior embodiment, wherein the average thickness of the layer of uniform thickness is about equal to a minimum dimension of an analyte in the sample.

The devices or methods of any prior embodiment, wherein the inter-spacer distance is in the range of 7 μm to 50 μm.

The devices or methods of any prior embodiment, wherein the inter-spacer distance is in the range of 50 μm to 120 μm.

The devices or methods of any prior embodiment, wherein the inter-spacer distance is in the range of 120 μm to 200 μm (micron).

The devices or methods of any prior embodiment, wherein the inter-spacer distance is substantially periodic.

The devices or methods of any prior embodiment, wherein the spacers are pillars with a cross-sectional shape selected from round, polygonal, circular, square, rectangular, oval, elliptical, or any combination of the same.

The devices or methods of any prior embodiment, wherein the spacers have a pillar shape and have a substantially flat top surface, wherein, for each spacer, the ratio of the lateral dimension of the spacer to its height is at least 1.

The devices or methods of any prior embodiment, wherein each spacer has the ratio of the lateral dimension of the spacer to its height is at least 1.

The devices or methods of any prior embodiment, wherein the minimum lateral dimension of spacer is less than or substantially equal to the minimum dimension of an analyte in the sample.

The devices or methods of any prior embodiment, wherein the minimum lateral dimension of spacer is in the range of 0.5 μm to 100 μm.

The devices or methods of any prior embodiment, wherein the minimum lateral dimension of spacer is in the range of 0.5 μm to 10 μm.

The devices or methods of any prior embodiment, wherein the sample is blood.

The devices or methods of any prior embodiment, wherein the sample is whole blood without dilution by liquid.

The devices or methods of any prior embodiment, wherein the sample is a biological sample selected from amniotic fluid, aqueous humour, vitreous humour, blood (e.g., whole blood, fractionated blood, plasma or serum), breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensates, sebum, semen, sputum, sweat, synovial fluid, tears, vomit, and urine.

The devices or methods of any prior embodiment, wherein the sample is a biological sample, an environmental sample, a chemical sample, or clinical sample.

The devices or methods of any prior embodiment, wherein the spacers have a pillar shape, and the sidewall corners of the spacers have a round shape with a radius of curvature at least 1 μm.

The devices or methods of any prior embodiment, wherein the spacers have a density of at least 100/mm².

The devices or methods of any prior embodiment, wherein the spacers have a density of at least 1,000/mm².

The devices or methods of any prior embodiment, wherein at least one of the plates is transparent.

The devices or methods of any prior embodiment, wherein at least one of the plates is made from a flexible polymer.

The devices or methods of any prior embodiment, wherein, for a pressure that compresses the plates, the spacers are not compressible and/or, independently, only one of the plates is flexible.

The device of any of any prior embodiment, wherein the flexible plate has a thickness in the range of 10 μm to 200 μm.

The devices or methods of any prior embodiment, wherein the variation is less than 30%.

The devices or methods of any prior embodiment, wherein the variation is less than 10%.

The devices or methods of any prior embodiment, wherein the variation is less than 5%.

The devices or methods of any prior embodiment, wherein the first and second plates are connected and are configured to be changed from the open configuration to the closed configuration by folding the plates.

The devices or methods of any prior embodiment, wherein the first and second plates are connected by a hinge and are configured to be changed from the open configuration to the closed configuration by folding the plates along the hinge.

The devices or methods of any prior embodiment, wherein the first and second plates are connected by a hinge that is a separate material to the plates, and are configured to be changed from the open configuration to the closed configuration by folding the plates along the hinge

The devices or methods of any prior embodiment, wherein the first and second plates are made in a single piece of material and are configured to be changed from the open configuration to the closed configuration by folding the plates.

The devices or methods of any prior embodiment, wherein the layer of uniform thickness sample is uniform over a lateral area that is at least 1 mm².

The devices or methods of any prior embodiment, wherein the device is configured to analyze the sample in 60 seconds or less.

The devices or methods of any prior embodiment, wherein at the closed configuration, the final sample thickness device is configured to analyze the sample in 60 seconds or less.

The devices or methods of any prior embodiment, wherein at the closed configuration, the final sample thickness device is configured to analyze the sample in 10 seconds or less.

The devices or methods of any prior embodiment, wherein the dry binding site comprises a capture agent.

The devices or methods of any prior embodiment, wherein the dry binding site comprises an antibody or nucleic acid.

The devices or methods of any prior embodiment, wherein the releasable dry reagent is a labeled reagent.

The devices or methods of any prior embodiment, wherein the releasable dry reagent is a fluorescently-labeled reagent.

The devices or methods of any prior embodiment, wherein the releasable dry reagent is a fluorescently-labeled antibody.

The devices or methods of any prior embodiment, wherein the releasable dry reagent is a cell stain.

The devices or methods of any prior embodiment, wherein the releasable dry reagent is a cell lysing.

The devices or methods of any prior embodiment, wherein the detector is an optical detector that detects an optical signal.

The devices or methods of any prior embodiment, wherein the detector is an electric detector that detect electrical signal.

The device of any prior device embodiment, wherein the spacing are fixed on a plate by directly embossing the plate or injection molding of the plate.

The device of any prior device embodiment, wherein the materials of the plate and the spacers are selected from polystyrene, PMMA, PC, COC, COP, or another plastic.

A system for rapidly analyzing a sample using a mobile phone comprising:

(a) a device of any prior embodiment;

(b) a mobile communication device comprising:
i. one or a plurality of cameras for the detecting and/or imaging the sample;
ii. electronics, signal processors, hardware and software for receiving and/or processing the detected signal and/or the image of the sample and for remote communication; and

(c) a light source from either the mobile communication device or an external source;

wherein the detector in The devices or methods of any prior embodiment is provided by the mobile communication device, and detects an analyte in the sample at the closed configuration.

The system of any prior system embodiment, wherein one of the plates has a binding site that binds an analyte, wherein at least part of the uniform sample thickness layer is over the binding site, and is substantially less than the average lateral linear dimension of the binding site.

The system of any prior system embodiment, further comprising a housing configured to hold the sample and to be mounted to the mobile communication device.

The system of any prior system embodiment, wherein the housing comprises optics for facilitating the imaging and/or signal processing of the sample by the mobile communication device, and a mount configured to hold the optics on the mobile communication device.

The system of any prior system embodiment, wherein an element of the optics in the housing is movable relative to the housing.

The system of any prior system embodiment, wherein the mobile communication device is configured to communicate test results to a medical professional, a medical facility or an insurance company.

The system of any prior system embodiment, wherein the mobile communication device is further configured to communicate information on the test and the subject with the medical professional, medical facility or insurance company.

The system of any prior system embodiment, wherein the mobile communication device is further configured to communicate information of the test to a cloud network, and the cloud network process the information to refine the test results.

The system of any prior system embodiment, wherein the mobile communication device is further configured to communicate information of the test and the subject to a cloud network, the cloud network process the information to refine the test results, and the refined test results will send back the subject.

The system of any prior system embodiment, wherein the mobile communication device is configured to receive a prescription, diagnosis or a recommendation from a medical professional.

The system of any prior system embodiment, wherein the mobile communication device is configured with hardware and software to:

(a) capture an image of the sample;
(b) analyze a test location and a control location in in image; and
(c) compare a value obtained from analysis of the test location to a threshold value that characterizes the rapid diagnostic test.

The system of any prior system embodiment, wherein at least one of the plates comprises a storage site in which assay reagents are stored.

The system of any prior system embodiment, at least one of the cameras reads a signal from the device.

The system of any prior system embodiment, wherein the mobile communication device communicates with the remote location via a wifi or cellular network.

The system of any prior system embodiment, wherein the mobile communication device is a mobile phone.

A method for rapidly analyzing an analyte in a sample using a mobile phone, comprising:

(a) depositing a sample on the device of any prior system embodiment;

(b) assaying an analyte in the sample deposited on the device to generate a result; and

(c) communicating the result from the mobile communication device to a location remote from the mobile communication device.

The method of any prior embodiments, wherein the analyte comprises a molecule (e.g., a protein, peptides, DNA, RNA, nucleic acid, or other molecule), cells, tissues, viruses, and nanoparticles with different shapes.

The method of any prior embodiment, wherein the analyte comprises white blood cell, red blood cell and platelets.

The method of any prior embodiment, wherein the assaying comprises performing a white blood cells differential assay.

The method of any prior embodiments, wherein the method comprises:

analyzing the results at the remote location to provide an analyzed result; and

communicating the analyzed result from the remote location to the mobile communication device.

The method of any prior embodiment, wherein the analysis is done by a medical professional at a remote location.

The method of any prior embodiment, wherein the mobile communication device receives a prescription, diagnosis or a recommendation from a medical professional at a remote location.

The method of any prior embodiment, wherein the sample is a bodily fluid.

The method of any prior embodiment, wherein the bodily fluid is blood, saliva or urine.

The method of any prior embodiment, wherein the sample is whole blood without dilution by a liquid.

The method of any prior embodiment, wherein the assaying step comprises detecting an analyte in the sample.

The method of any prior embodiment, wherein the analyte is a biomarker.

The method of any prior embodiment, wherein the analyte is a protein, nucleic acid, cell, or metabolite.

The method of any prior embodiment, wherein the method comprises counting the number of red blood cells.

The method of any of any prior embodiment, wherein the method comprises counting the number of white blood cells.

The method of any prior embodiment, wherein method comprises staining the cells in the sample and counting the number of neutrophils, lymphocytes, monocytes, eosinophils and basophils.

The method of any prior embodiments embodiment, wherein the assay done in step (b) is a binding assay or a biochemical assay.

A method for analyzing a sample comprising:

obtaining a device of any prior device embodiment;
depositing the sample onto one or both pates of the device;
placing the plates in a closed configuration and applying an external force over at least part of the plates; and analyzing the layer of uniform thickness while the plates are the closed configuration.

The devices or methods of any prior embodiment, wherein the first plate further comprises, on its surface, a first predetermined assay site and a second predetermined assay site, wherein the distance between the edges of the assay site is substantially larger than the thickness of the uniform thickness layer when the plates are in the closed position, wherein at least a part of the uniform thickness layer is over the predetermined assay sites, and wherein the sample has one or a plurality of analytes that are capable of diffusing in the sample.

The devices or methods of any prior embodiment, wherein the first plate has, on its surface, at least three analyte assay sites, and the distance between the edges of any two neighboring assay sites is substantially larger than the thickness of the uniform thickness layer when the plates are in the closed position, wherein at least a part of the uniform thickness layer is over the assay sites, and wherein the sample has one or a plurality of analytes that are capable of diffusing in the sample.

The devices or methods of any prior embodiment, wherein the first plate has, on its surface, at least two neighboring analyte assay sites that are not separated by a distance that is substantially larger than the thickness of the uniform thickness layer when the plates are in the closed position, wherein at least a part of the uniform thickness layer is over the assay sites, and wherein the sample has one or a plurality of analytes that are capable of diffusing in the sample.

The devices or methods of any prior embodiment, wherein the analyte assay area is between a pair of electrodes.

The devices or methods of any prior embodiment, wherein the assay area is defined by a patch of dried reagent.

The devices or methods of any prior embodiment, wherein the assay area binds to and immobilizes the analyte

The devices or methods of any prior embodiment, wherein the assay area is defined by a patch of binding reagent that, upon contacting the sample, dissolves into the sample, diffuses in the sample, and binds to the analyte.

The devices or methods of any prior embodiment, wherein the inter-spacer distance is in the range of 14 μm to 200 μm.

The devices or methods of any prior embodiment, wherein the inter-spacer distance is in the range of 7 μm to 20 μm.

The devices or methods of any prior embodiment, wherein the spacers are pillars with a cross-sectional shape selected from round, polygonal, circular, square, rectangular, oval, elliptical, or any combination of the same.

The devices or methods of any prior embodiment, wherein the spacers have are pillar shape and have a substantially flat top surface, wherein, for each spacer, the ratio of the lateral dimension of the spacer to its height is at least 1.

The devices or methods of any prior embodiment, wherein the spacers have a pillar shape, and the sidewall corners of the spacers have a round shape with a radius of curvature at least 1 μm.

The devices or methods of any prior embodiment, wherein the spacers have a density of at least 1,000/mm².

The devices or methods of any prior embodiment, wherein at least one of the plates is transparent.

The devices or methods of any prior embodiment, wherein at least one of the plates is made from a flexible polymer.

The devices or methods of any prior embodiment, wherein only one of the plates is flexible.

The device of any prior embodiment, wherein the area-determination device is a camera.

The device of any prior embodiment, wherein the area-determination device comprises an area in the sample contact area of a plate, wherein the area is less than 1/100, 1/20, 1/10, ⅙, ⅕, ¼, ⅓, ½, ⅔ of the sample contact area, or in a range between any of the two values.

The device of any prior embodiment, wherein the area-determination device comprises a camera and an area in the sample contact area of a plate, wherein the area is in contact with the sample.

The devices or methods of any prior embodiment, wherein the deformable sample comprises a liquid sample.

The devices or methods of any prior embodiment, wherein the imprecision force has a variation at least 30% of the total force that actually is applied.

The devices or methods of any prior embodiment, wherein the imprecision force has a variation at least 20%, 30%, 40%, 50%, 60, 70%, 80%, 90%, 100%, 150%, 200%, 300%, 500%, or in a range of any two values, of the total force that actually is applied.

The device of any prior embodiment, wherein spacers have a flat top.

The device of any prior embodiment, wherein the device is further configured to have, after the pressing force is removed, a sample thickness that is substantially the same in thickness and uniformity as that when the force is applied.

The device of any prior embodiment, wherein the imprecise force is provided by human hand.

The device of any prior embodiment, wherein the inter spacer distance is substantially constant.

The device of any prior embodiment, wherein the inter spacer distance is substantially periodic in the area of the uniform sample thickness area.

The device of any prior embodiment, wherein the multiplication product of the filling factor and the Young's modulus of the spacer is 2 MPa or larger.

The device of any prior embodiment, wherein the force is applied by hand directly or indirectly.

The device of any prior embodiment, wherein the force applied is in the range of 1 N to 20 N.

The device of any prior embodiment, wherein the force applied is in the range of 20 N to 200 N.

The device of any prior embodiment wherein the highly uniform layer has a thickness that varies by less than 15%, 10%, or 5% of an average thickness.

The device of any prior embodiment, wherein the imprecise force is applied by pinching the device between a thumb and forefinger.

The device of any prior embodiment, wherein the predetermined sample thickness is larger than the spacer height.

The device of any prior embodiment, wherein the device holds itself in the closed configuration after the pressing force has been removed.

The device of any prior embodiment, wherein the uniform thickness sample layer area is larger than that area upon which the pressing force is applied.

The device of any prior embodiment, wherein the spacers do not significantly deform during application of the pressing force.

The device of any prior embodiment, wherein the pressing force is not predetermined beforehand and is not measured.

In some embodiments, the fluidic sample is replaced by a deformable sample and the embodiments for making at least a part of the fluidic sample into a uniform thickness layer can make at least a part of the deformable sample into a uniform thickness layer.

The devices and methods of any prior device claim, wherein the inter spacer distance is periodic.

The devices and methods of any prior device claim, wherein the spacers have a flat top.

The devices and methods of any prior device claim, wherein the inter spacer distance is at least two times large than the size of the targeted analyte in the sample.

### Manufacturing of Q-Card

In certain embodiments, an embodiment of the Q-Card comprising: a first plate, a second plate, and a hinge, wherein
i. the first plate, that is about 200 nm to 1500 nm thick, comprises, on its inner surface, (a) a sample contact area for contacting a sample, and (b) a sample overflow dam that surrounds the sample contact area is configured to present a sample flow outside of the dam;
ii. the second plate is 10 μm to 250 μm thick and comprises, on its inner surface, (a) a sample contact area for contacting a sample, and (b) spacers on the sample contact area;
iii. the hinge that connect the first and the second plates; and
   wherein the first and second plate are movable relative to each other around the axis of the hinge.

In certain embodiments, an embodiment of the Q-Card comprising: a first plate, a second plate, and a hinge, wherein
i. the first plate, that is about 200 nm to 1500 nm thick, comprises, on its inner surface, (a) a sample contact area for contacting a sample, (b) a sample overflow dam that surrounds the sample contact area is configured to present a sample flow outside of the dam, and (c) spacers on the sample contact area;
ii. the second plate, that is 10 μm to 250 μm thick, comprises, on its inner surface, a sample contact area for contacting a sample;
iii. the hinge that connect the first and the second plates; and
   wherein the first and second plate are movable relative to each other around the axis of the hinge.

In certain embodiments, an embodiment of the Q-Card comprising: a first plate, a second plate, and a hinge, wherein
i. the first plate, that is about 200 nm to 1500 nm thick, comprises, on its inner surface, (a) a sample contact area for contacting a sample, and (b) spacers on the sample contact area;
ii. the second plate, that is 10 μm to 250 μm thick, comprises, on its inner surface, (a) a sample contact area for contacting a sample, and (b) a sample overflow dam that surrounds the sample contact area is configured to present a sample flow outside of the dam, and;
iii. the hinge that connect the first and the second plates; and
   wherein the first and second plate are movable relative to each other around the axis of the hinge.

In certain embodiments, an embodiment of the Q-Card comprising: a first plate, a second plate, and a hinge, wherein
i. the first plate, that is about 200 nm to 1500 nm thick, comprises, on its inner surface, a sample contact area for contacting a sample;
ii. the second plate, that is 10 μm to 250 μm thick, comprises, on its inner surface, (a) a sample contact area for contacting a sample, (b) a sample overflow dam that surrounds the sample contact area is configured to present a sample flow outside of the dam, and (c) spacers on the sample contact area; and
iii. the hinge that connect the first and the second plates; and
   wherein the first and second plate are movable relative to each other around the axis of the hinge.

In certain embodiments, an embodiment of a method for fabricating the Q-Card of any embodiments of any prior method, comprising:

(a) injection molding of the first plate,

(b) nanoimprinting or extrusion printing of the second plate.

In certain embodiments, an embodiment of a method for fabricating the Q-Card of any embodiments of any prior method, comprising:

(a) Laser cutting the first plate,

(b) nanoimprinting or extrusion printing of the second plate.

In certain embodiments, an embodiment of a method for fabricating the Q-Card of any embodiments of any prior method, comprising:

(a) Injection molding and laser cutting the first plate,

(b) nanoimprinting or extrusion printing of the second plate.

In certain embodiments, an embodiment of a method for fabricating the Q-Card of any embodiments of any prior method, comprising: nanoimprinting or extrusion printing to fabricated both the first and the second plate.

In certain embodiments, an embodiment of a method for fabricating the Q-Card of any embodiments of any prior method, comprising: fabricating the first plate or the second plate, using injection molding, laser cutting the first plate, nanoimprinting, extrusion printing, or a combination of thereof.

The method of any embodiments of any prior method, wherein the method further comprises a step of attach the hinge on the first and the second plates after the fabrication of the first and second plates.

### Compressed Regulated Open Flow” (CROF)

In assaying, a manipulation of a sample or a reagent can lead to improvements in the assaying. The manipulation includes, but not limited to, manipulating the geometric shape and location of a sample and/or a reagent, a mixing or a binding of a sample and a reagent, and a contact area of a sample of reagent to a plate.

Many embodiments of the present invention manipulate the geometric size, location, contact areas, and mixing of a sample and/or a reagent using a method, termed “compressed regulated open flow (CROF)”, and a device that performs CROF.

The term “compressed open flow (COF)” refers to a method that changes the shape of a flowable sample deposited on a plate by (i) placing other plate on top of at least a part of the sample and (ii) then compressing the sample between two plates by pushing the two plates towards each other; wherein the compression reduces a thickness of at least a part of the sample and makes the sample flow into open spaces between the plates.

The term “compressed regulated open flow” or “CROF” (or “self-calibrated compressed open flow” or “SCOF” or “SCCOF”) refers to a particular type of COF, wherein the final thickness of a part or entire sample after the compression is “regulated” by spacers, wherein the spacers, that are placed between the two plates.

The term “the final thickness of a part or entire sample is regulated by spacers” in a CROF means that during a CROF, once a specific sample thickness is reached, the relative movement of the two plates and hence the change of sample thickness stop, wherein the specific thickness is determined by the spacer.

One embodiment of the method of CROF, as illustrated in FIG. A1, comprises:

(a) obtaining a sample, that is flowable;

(b) obtaining a first plate and a second plate that are movable relative to each other into different configurations, wherein each plate has a sample contact surface that is substantially planar, wherein one or both of the plates comprise spacers and the spacers have a predetermined height, and the spacers are on a respective sample contacting surface;

(c) depositing, when the plates are configured in an open configuration, the sample on one or both of the plates; wherein the open configuration is a configuration in which the two plates are either partially or completely separated apart and the spacing between the plates is not regulated by the spacers; and

(d) after (c), spreading the sample by bringing the plates into a closed configuration, wherein, in the closed configuration: the plates are facing each other, the spacers and a relevant volume of the sample are between the plates, the thickness of the relevant volume of the sample is regulated by the plates and the spacers, wherein the relevant volume is at least a portion of an entire volume of the sample, and wherein during the sample spreading, the sample flows laterally between the two plates.

### Related Documents and

The present invention includes a variety of embodiments, which can be combined in multiple ways as long as the various components do not contradict one another. The embodiments should be regarded as a single invention file: each filing has other filing as the references and is also referenced in its entirety and for all purpose, rather than as a discrete independent. These embodiments include not only the disclosures in the current file, but also the documents that are herein referenced, incorporated, or to which priority is claimed.

### (1) Definitions

The terms used in describing the devices/apparatus, systems, and methods herein disclosed are defined in the current application, or in PCT Application (designating U.S.) Nos. PCT/US2016/046437 and PCT/US2016/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

The terms “CROF Card (or card)”, “COF Card”, “QMAX-Card”, “Q-Card”, “CROF device”, “COF device”, “QMAX-device”, “CROF plates”, “COF plates”, and “QMAX-plates” are interchangeable, except that in some embodiments, the COF card does not comprise spacers; and the terms refer to a device that comprises a first plate and a second plate that are movable relative to each other into different configurations (including an open configuration and a closed configuration), and that comprises spacers (except some embodiments of the COF card) that regulate the spacing between the plates. The term “X-plate” refers to one of the two plates in a CROF card, wherein the spacers are fixed to this plate. More descriptions of the COF Card, CROF Card, and X-plate are given in the provisional application Ser. No. 62/456,065, filed on Feb. 7, 2017, which is incorporated herein in its entirety for all purposes.

### (2) Sample

The devices/apparatus, systems, and methods herein disclosed can be applied to manipulation and detection of various types of samples. The samples are herein disclosed, listed, described, and/or summarized in PCT Application (designating U.S.) Nos. PCT/US2016/046437 and PCT/US2016/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

The devices, apparatus, systems, and methods herein disclosed can be used for samples such as but not limited to diagnostic samples, clinical samples, environmental samples and foodstuff samples. The types of sample include but are not limited to the samples listed, described and/or summarized in PCT Application (designating U.S.) Nos. PCT/US2016/046437 and PCT/US2016/051775, which were respectively filed on Aug. 10, 2016 and Sep. 14, 2016, and are hereby incorporated by reference by their entireties.

For example, in some embodiments, the devices, apparatus, systems, and methods herein disclosed are used for a sample that includes cells, tissues, bodily fluids and/or a mixture thereof. In some embodiments, the sample comprises a human body fluid. In some embodiments, the sample comprises at least one of cells, tissues, bodily fluids, stool, amniotic fluid, aqueous humour, vitreous humour, blood, whole blood, fractionated blood, plasma, serum, breast milk, cerebrospinal fluid, cerumen, chyle, chime, endolymph, perilymph, feces, gastric acid, gastric juice, lymph, mucus, nasal drainage, phlegm, pericardial fluid, peritoneal fluid, pleural fluid, pus, rheum, saliva, sebum, semen, sputum, sweat, synovial fluid, tears, vomit, urine, and exhaled breath condensate.

In some embodiments, the devices, apparatus, systems, and methods herein disclosed are used for an environmental sample that is obtained from any suitable source, such as but not limited to: river, lake, pond, ocean, glaciers, icebergs, rain, snow, sewage, reservoirs, tap water, drinking water, etc.; solid samples from soil, compost, sand, rocks, concrete, wood, brick, sewage, etc.; and gaseous samples from the air, underwater heat vents, industrial exhaust, vehicular exhaust, etc. In certain embodiments, the environmental sample is fresh from the source; in certain embodiments, the environmental sample is processed. For example, samples that are not in liquid form are converted to liquid form before the subject devices, apparatus, systems, and methods are applied.

In some embodiments, the devices, apparatus, systems, and methods herein disclosed are used for a foodstuff sample, which is suitable or has the potential to become suitable for animal consumption, e.g., human consumption. In some embodiments, a foodstuff sample includes raw ingredients, cooked or processed food, plant and animal sources of food, preprocessed food as well as partially or fully processed food, etc. In certain embodiments, samples that are not in liquid form are converted to liquid form before the subject devices, apparatus, systems, and methods are applied.

In some embodiments, the volume of the sample includes, but is not limited to, about 100 μL or less, 75 μL or less, 50 μL or less, 25 μL or less, 20 μL or less, 15 μL or less, 10 μL or less, 5 μL or less, 3 μL or less, 1 μL or less, 0.5 μL or less, 0.1 μL or less, 0.05 μL or less, 0.001 μL or less, 0.0005 μL or less, 0.0001 μL or less, 10 μL or less, 1 μL or less, or a range between any two of the values. In some embodiments, the volume of the sample includes, but is not limited to, about 10 μL or less, 5 μL or less, 3 μL or less, 1 μL or less, 0.5 μL or less, 0.1 μL or less, 0.05 μL or less, 0.001 μL or less, 0.0005 μL or less, 0.0001 μL or less, 10 μL or less, 1 μL or less, or a range between any two of the values.

In some embodiments, the amount of the sample is about a drop of liquid. In certain embodiments, the amount of sample is the amount collected from a pricked finger or fingerstick.

In certain embodiments, the amount of sample is the amount collected from a microneedle, micropipette or a venous draw.

In certain embodiments, the sample holder is configured to hold a fluidic sample. In certain embodiments, the sample holder is configured to compress at least part of the fluidic sample into a thin layer. In certain embodiments, the sample holder comprises structures that are configured to heat and/or cool the sample. In certain embodiments, the heating source provides electromagnetic waves that can be absorbed by certain structures in the sample holder to change the temperature of the sample. In certain embodiments, the signal sensor is configured to detect and/or measure a signal from the sample. In certain embodiments, the signal sensor is configured to detect and/or measure an analyte in the sample. In certain embodiments, the heat sink is configured to absorb heat from the sample holder and/or the heating source. In certain embodiments, the heat sink comprises a chamber that at least partly enclose the sample holder.

### (3) Q-Card, Spacers and Uniform Sample Thickness

The devices/apparatus, systems, and methods herein disclosed can include or use Q-cards, spacers, and uniform sample thickness embodiments for sample detection, analysis, and quantification. In some embodiments, the Q-card comprises spacers, which help to render at least part of the sample into a layer of high uniformity. The structure, material, function, variation and dimension of the spacers, as well as the uniformity of the spacers and the sample layer, are herein disclosed, listed, described, and/or summarized in PCT Application (designating U.S.) Nos. PCT/US2016/046437 and PCT/US2016/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

The term “open configuration” of the two plates in a QMAX process means a configuration in which the two plates are either partially or completely separated apart and the spacing between the plates is not regulated by the spacers

The term “closed configuration” of the two plates in a QMAX process means a configuration in which the plates are facing each other, the spacers and a relevant volume of the sample are between the plates, the relevant spacing between the plates, and thus the thickness of the relevant volume of the sample, is regulated by the plates and the spacers, wherein the relevant volume is at least a portion of an entire volume of the sample.

The term “a sample thickness is regulated by the plate and the spacers” in a QMAX process means that for a give condition of the plates, the sample, the spacer, and the plate compressing method, the thickness of at least a port of the sample at the closed configuration of the plates can be predetermined from the properties of the spacers and the plate.

The term “inner surface” or “sample surface” of a plate in a QMAX card refers to the surface of the plate that touches the sample, while the other surface (that does not touch the sample) of the plate is termed “outer surface”.

The term “height” or “thickness” of an object in a QMAX process refers to, unless specifically stated, the dimension of the object that is in the direction normal to a surface of the plate. For example, spacer height is the dimension of the spacer in the direction normal to a surface of the plate, and the spacer height and the spacer thickness means the same thing.

The term “area” of an object in a QMAX process refers to, unless specifically stated, the area of the object that is parallel to a surface of the plate. For example, spacer area is the area of the spacer that is parallel to a surface of the plate.

The term of QMAX card refers the device that perform a QMAX (e.g. CROF) process on a sample, and have or not have a hinge that connect the two plates.

The term “QMAX card with a hinge and “QMAX card” are interchangeable.

The term “angle self-maintain”, “angle self-maintaining”, or “rotation angle self-maintaining” refers to the property of the hinge, which substantially maintains an angle between the two plates, after an external force that moves the plates from an initial angle into the angle is removed from the plates.

In using QMAX card, the two plates need to be open first for sample deposition. However, in some embodiments, the QMAX card from a package has the two plates are in contact each other (e.g. a close position), and to separate them is challenges, since one or both plates are very thing. To facilitate an opening of the QMAX card, opening notch or notches are created at the edges or corners of the first plate or both places, and, at the close position of the plates, a part of the second plate placed over the opening notch, hence in the notch of the first plate, the second plate can be lifted open without a blocking of the first plate.

In the QMAX assay platform, a QMAX card uses two plates to manipulate the shape of a sample into a thin layer (e.g. by compressing). In certain embodiments, the plate manipulation needs to change the relative position (termed: plate configuration) of the two plates several times by human hands or other external forces. There is a need to design the QMAX card to make the hand operation easy and fast.

In QMAX assays, one of the plate configurations is an open configuration, wherein the two plates are completely or partially separated (the spacing between the plates is not controlled by spacers) and a sample can be deposited. Another configuration is a closed configuration, wherein at least part of the sample deposited in the open configuration is compressed by the two plates into a layer of highly uniform thickness, the uniform thickness of the layer is confined by the inner surfaces of the plates and is regulated by the plates and the spacers. In some embodiments, the average spacing between the two plates is more than 300 μm.

In a QMAX assay operation, an operator needs to first make the two plates to be in an open configuration ready for sample deposition, then deposit a sample on one or both of the plates, and finally close the plates into a close position. In certain embodiments, the two plates of a QMAX card are initially on top of each other and need to be separated to get into an open configuration for sample deposition. When one of the plate is a thin plastic film (175 μm thick PMA), such separation can be difficult to perform by hand. The present invention intends to provide the devices and methods that make the operation of certain assays, such as the QMAX card assay, easy and fast.

In some embodiments, the QMAX device comprises a hinge that connect two or more plates together, so that the plates can open and close in a similar fashion as a book. In some embodiments, the material of the hinge is such that the hinge can self-maintain the angle between the plates after adjustment. In some embodiments, the hinge is configured to maintain the QMAX card in the closed configuration, such that the entire QMAX card can be slide in and slide out a card slot without causing accidental separation of the two plates. In some embodiments, the QMAX device comprises one or more hinges that can control the rotation of more than two plates.

In some embodiments, the hinge is made from a metallic material that is selected from a group consisting of gold, silver, copper, aluminum, iron, tin, platinum, nickel, cobalt, alloys, or any combination of thereof. In some embodiments, the hinge comprises a single layer, which is made from a polymer material, such as but not limited to plastics. The polymer material is selected from the group consisting of acrylate polymers, vinyl polymers, olefin polymers, cellulosic polymers, noncellulosic polymers, polyester polymers, Nylon, cyclic olefin copolymer (COC), poly(methyl methacrylate) (PMMB), polycarbonate (PC), cyclic olefin polymer (COP), liquid crystalline polymer (LCP), polyamide (PB), polyethylene (PE), polyimide (PI), polypropylene (PP), poly(phenylene ether) (PPE), polystyrene (PS), polyoxymethylene (POM), polyether ether ketone (PEEK), polyether sulfone (PES), poly(ethylene phthalate) (PET), polytetrafluoroethylene (PTFE), polyvinyl chloride (PVC), polyvinylidene fluoride (PVDF), polybutylene terephthalate (PBT), fluorinated ethylene propylene (FEP), perfluoroalkoxyalkane (PFB), polydimethylsiloxane (PDMS), rubbers, or any combinations of thereof. In some embodiments, the polymer material is selected from polystyrene, PMMB, PC, COC, COP, other plastic, or any combination of thereof.

In essence, the term “spacers” or “stoppers” refers to, unless stated otherwise, the mechanical objects that set, when being placed between two plates, a limit on the minimum spacing between the two plates that can be reached when compressing the two plates together. Namely, in the compressing, the spacers will stop the relative movement of the two plates to prevent the plate spacing becoming less than a preset (i.e. predetermined) value.

The term “a spacer has a predetermined height” and “spacers have a predetermined inter-spacer distance” means, respectively, that the value of the spacer height and the inter spacer distance is known prior to a QMAX process. It is not predetermined, if the value of the spacer height and the inter-spacer distance is not known prior to a QMAX process. For example, in the case that beads are sprayed on a plate as spacers, where beads are landed at random locations of the plate, the inter-spacer distance is not predetermined. Another example of not predetermined inter spacer distance is that the spacers moves during a QMAX processes.

The term “a spacer is fixed on its respective plate” in a QMAX process means that the spacer is attached to a location of a plate and the attachment to that location is maintained during a QMAX (i.e. the location of the spacer on respective plate does not change) process. An example of “a spacer is fixed with its respective plate” is that a spacer is monolithically made of one piece of material of the plate, and the location of the spacer relative to the plate surface does not change during the QMAX process. An example of “a spacer is not fixed with its respective plate” is that a spacer is glued to a plate by an adhesive, but during a use of the plate, during the QMAX process, the adhesive cannot hold the spacer at its original location on the plate surface and the spacer moves away from its original location on the plate surface.

In some embodiments, human hands can be used to press the plates into a closed configuration; In some embodiments, human hands can be used to press the sample into a thin layer. The manners in which hand pressing is employed are described and/or summarized in PCT Application (designating U.S.) Nos. PCT/US2016/046437 filed on Aug. 10, 2016 and PCT/US2016/051775 filed on Sep. 14, 2016, and in US Provisional Application Nos. 62/431,639 filed on Dec. 9, 2016, 62/456,287 filed on Feb. 8, 2017, 62/456,065 filed on Feb. 7, 2017, 62/456,504 filed on Feb. 8, 2017, and 62/460,062 filed on Feb. 16, 2017, which are all hereby incorporated by reference by their entireties.

In some embodiments, human hand can be used to manipulate or handle the plates of the QMAX device. In certain embodiments, the human hand can be used to apply an imprecise force to compress the plates from an open configuration to a closed configuration. In certain embodiments, the human hand can be used to apply an imprecise force to achieve high level of uniformity in the thickness of the sample (e.g. less than 5%, 10%, 15%, or 20% variability).

### (4) Hinges, Opening Notches, Recessed Edge and Sliders

The devices/apparatus, systems, and methods herein disclosed can include or use Q-cards for sample detection, analysis, and quantification. In some embodiments, the Q-card comprises hinges, notches, recesses, and sliders, which help to facilitate the manipulation of the Q card and the measurement of the samples. The structure, material, function, variation and dimension of the hinges, notches, recesses, and sliders are herein disclosed, listed, described, and/or summarized in PCT Application (designating U.S.) Nos. PCT/US2016/046437 and PCT/US2016/051775, which were respectively filed on Aug. 10, 2016 and Sep. 14, 2016, U.S. Provisional Application No. 62/431,639, which was filed on Dec. 9, 2016, U.S. Provisional Application No. 62/456,065, which was filed on Feb. 7, 2017, U.S. Provisional Application Nos. 62/456,287 and 62/456,504, which was filed on Feb. 8, 2017, and U.S. Provisional Application No. 62/539,660, which was filed on Aug. 1, 2017, all of which applications are incorporated herein in their entireties for all purposes.

In some embodiments, the QMAX device comprises opening mechanisms such as but not limited to notches on plate edges or strips attached to the plates, making is easier for a user to manipulate the positioning of the plates, such as but not limited to separating the plates of by hand.

In some embodiments, the QMAX device comprises trenches on one or both of the plates. In certain embodiments, the trenches limit the flow of the sample on the plate.

### (5) Q-Card and Adaptor

The devices/apparatus, systems, and methods herein disclosed can include or use Q-cards for sample detection, analysis, and quantification. In some embodiments, the Q-card is used together with an adaptor that is configured to accommodate the Q-card and connect to a mobile device so that the sample in the Q-card can be imaged, analyzed, and/or measured by the mobile device. The structure, material, function, variation, dimension and connection of the Q-card, the adaptor, and the mobile are herein disclosed, listed, described, and/or summarized in PCT Application (designating U.S.) Nos. PCT/US2016/046437 and PCT/US2016/051775, which were respectively filed on Aug. 10, 2016 and Sep. 14, 2016, U.S. Provisional Application No. 62/456,065, which was filed on Feb. 7, 2017, U.S. Provisional Application Nos. 62/456,287 and 62/456,590, which were filed on Feb. 8, 2017, U.S. Provisional Application No. 62/456,504, which was filed on Feb. 8, 2017, U.S. Provisional Application No. 62/459,544, which was filed on Feb. 15, 2017, and U.S. Provisional Application Nos. 62/460,075 and 62/459,920, which were filed on Feb. 16, 2017, all of which applications are incorporated herein in their entireties for all purposes.

In some embodiments, the adaptor comprises a receptacle slot, which is configured to accommodate the QMAX device when the device is in a closed configuration. In certain embodiments, the QMAX device has a sample deposited therein and the adaptor can be connected to a mobile device (e.g. a smartphone) so that the sample can be read by the mobile device. In certain embodiments, the mobile device can detect and/or analyze a signal from the sample. In certain embodiments, the mobile device can capture images of the sample when the sample is in the QMAX device and positioned in the field of view (FOV) of a camera, which in certain embodiments, is part of the mobile device.

In some embodiments, the adaptor comprises optical components, which are configured to enhance, magnify, and/or optimize the production of the signal from the sample. In some embodiments, the optical components include parts that are configured to enhance, magnify, and/or optimize illumination provided to the sample. In certain embodiments, the illumination is provided by a light source that is part of the mobile device. In some embodiments, the optical components include parts that are configured to enhance, magnify, and/or optimize a signal from the sample.

### (6) Smartphone Detection System

The devices/apparatus, systems, and methods herein disclosed can include or use Q-cards for sample detection, analysis, and quantification. In some embodiments, the Q-card is used together with an adaptor that can connect the Q-card with a smartphone detection system. In some embodiments, the smartphone comprises a camera and/or an illumination source The smartphone detection system, as well the associated hardware and software are herein disclosed, listed, described, and/or summarized in PCT Application (designating U.S.) Nos. PCT/US2016/046437 and PCT/US2016/051775, which were respectively filed on Aug. 10, 2016 and Sep. 14, 2016, U.S. Provisional Application No. 62/456,065, which was filed on Feb. 7, 2017, U.S. Provisional Application Nos. 62/456,287 and 62/456,590, which were filed on Feb. 8, 2017, U.S. Provisional Application No. 62/456,504, which was filed on Feb. 8, 2017, U.S. Provisional Application No. 62/459,544, which was filed on Feb. 15, 2017, and U.S. Provisional Application Nos. 62/460,075 and 62/459,920, which were filed on Feb. 16, 2017, all of which applications are incorporated herein in their entireties for all purposes.

In some embodiments, the smartphone comprises a camera, which can be used to capture images or the sample when the sample is positioned in the field of view of the camera (e.g. by an adaptor). In certain embodiments, the camera includes one set of lenses (e.g. as in iPhone™ 6). In certain embodiments, the camera includes at least two sets of lenses (e.g. as in iPhone™ 7). In some embodiments, the smartphone comprises a camera, but the camera is not used for image capturing.

In some embodiments, the smartphone comprises a light source such as but not limited to LED (light emitting diode). In certain embodiments, the light source is used to provide illumination to the sample when the sample is positioned in the field of view of the camera (e.g. by an adaptor). In some embodiments, the light from the light source is enhanced, magnified, altered, and/or optimized by optical components of the adaptor.

In some embodiments, the smartphone comprises a processor that is configured to process the information from the sample. The smartphone includes software instructions that, when executed by the processor, can enhance, magnify, and/or optimize the signals (e.g. images) from the sample. The processor can include one or more hardware components, such as a central processing unit (CPU), an application-specific integrated circuit (ASIC), an application-specific instruction-set processor (ASIP), a graphics processing unit (GPU), a physics processing unit (PPU), a digital signal processor (DSP), a field-programmable gate array (FPGA), a programmable logic device (PLD), a controller, a microcontroller unit, a reduced instruction-set computer (RISC), a microprocessor, or the like, or any combination thereof.

In some embodiments, the smartphone comprises a communication unit, which is configured and/or used to transmit data and/or images related to the sample to another device. Merely by way of example, the communication unit can use a cable network, a wireline network, an optical fiber network, a telecommunications network, an intranet, the Internet, a local area network (LAN), a wide area network (WAN), a wireless local area network (WLAN), a metropolitan area network (MAN), a wide area network (WAN), a public telephone switched network (PSTN), a Bluetooth network, a ZigBee network, a near field communication (NFC) network, or the like, or any combination thereof.

In some embodiments, the smartphone is an iPhone™, an Android™ phone, or a Windows™ phone.

### (7) Detection Methods

The devices/apparatus, systems, and methods herein disclosed can include or be used in various types of detection methods. The detection methods are herein disclosed, listed, described, and/or summarized in PCT Application (designating U.S.) Nos. PCT/US2016/046437 and PCT/US2016/051775, which were respectively filed on Aug. 10, 2016 and Sep. 14, 2016, U.S. Provisional Application No. 62/456,065, which was filed on Feb. 7, 2017, U.S. Provisional Application Nos. 62/456,287, 62/456,528, 62/456,631, 62/456,522, 62/456,598, 62/456,603, and 62/456,628, which were filed on Feb. 8, 2017, U.S. Provisional Application Nos. 62/459,276, 62/456,904, 62/457,075, and 62/457,009, which were filed on Feb. 9, 2017, and U.S. Provisional Application Nos. 62/459,303, 62/459,337, and 62/459,598, which were filed on Feb. 15, 2017, and U.S. Provisional Application Nos. 62/460,083, 62/460,076, which were filed on Feb. 16, 2017, all of which applications are incorporated herein in their entireties for all purposes.

### (8) Labels, Capture Agent and Detection Agent

The devices/apparatus, systems, and methods herein disclosed can employ various types of labels, capture agents, and detection agents that are used for analytes detection. The labels are herein disclosed, listed, described, and/or summarized in PCT Application (designating U.S.) Nos. PCT/US2016/046437 and PCT/US2016/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

I

In any embodiment, the QMAX device can contain a plurality of capture agents and/or detection agents that each bind to a biomarker selected from Tables B1, B2, B3 and/or B7 in U.S. Provisional Application No. 62/234,538 and/or PCT Application No. PCT/US2016/054025, wherein the reading step d) includes obtaining a measure of the amount of the plurality of biomarkers in the sample, and wherein the amount of the plurality of biomarkers in the sample is diagnostic of a disease or condition.

In any embodiment, the capture agent and/or detection agents can be an antibody epitope and the biomarker can be an antibody that binds to the antibody epitope. In some embodiments, the antibody epitope includes a biomolecule, or a fragment thereof, selected from Tables B4, B5 or B6 in U.S. Provisional Application No. 62/234,538 and/or PCT Application No. PCT/US2016/054025. In some embodiments, the antibody epitope includes an allergen, or a fragment thereof, selected from Table B5. In some embodiments, the antibody epitope includes an infectious agent-derived biomolecule, or a fragment thereof, selected from Table B6 in U.S. Provisional Application No. 62/234,538 and/or PCT Application No. PCT/US2016/054025.

In any embodiment, the QMAX device can contain a plurality of antibody epitopes selected from Tables B4, B5 and/or B6 in U.S. Provisional Application No. 62/234,538 and/or PCT Application No. PCT/US2016/054025, wherein the reading step d) includes obtaining a measure of the amount of a plurality of epitope-binding antibodies in the sample, and wherein the amount of the plurality of epitope-binding antibodies in the sample is diagnostic of a disease or condition.

### (9) Analytes

The devices/apparatus, systems, and methods herein disclosed can be applied to manipulation and detection of various types of analytes (including biomarkers). The analytes are herein disclosed, listed, described, and/or summarized in PCT Application (designating U.S.) Nos. PCT/US2016/046437 and PCT/US2016/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

The devices, apparatus, systems, and methods herein disclosed can be used for the detection, purification and/or quantification of various analytes. In some embodiments, the analytes are biomarkers that associated with various diseases. In some embodiments, the analytes and/or biomarkers are indicative of the presence, severity, and/or stage of the diseases. The analytes, biomarkers, and/or diseases that can be detected and/or measured with the devices, apparatus, systems, and/or method of the present invention include the analytes, biomarkers, and/or diseases listed, described and/or summarized in PCT Application (designating U.S.) Nos. PCT/US2016/046437 filed on Aug. 10, 2016, and PCT Application No. PCT/US2016/054025 filed on Sep. 27, 2016, and U.S. Provisional Application Nos. 62/234,538 filed on Sep. 29, 2015, 62/233,885 filed on Sep. 28, 2015, 62/293,188 filed on Feb. 9, 2016, and 62/305,123 filed on Mar. 8, 2016, which are all hereby incorporated by reference by their entireties. For example, the devices, apparatus, systems, and methods herein disclosed can be used in (a) the detection, purification and quantification of chemical compounds or biomolecules that correlates with the stage of certain diseases, e.g., infectious and parasitic disease, injuries, cardiovascular disease, cancer, mental disorders, neuropsychiatric disorders and organic diseases, e.g., pulmonary diseases, renal diseases, (b) the detection, purification and quantification of microorganism, e.g., virus, fungus and bacteria from environment, e.g., water, soil, or biological samples, e.g., tissues, bodily fluids, (c) the detection, quantification of chemical compounds or biological samples that pose hazard to food safety or national security, e.g. toxic waste, anthrax, (d) quantification of vital parameters in medical or physiological monitor, e.g., glucose, blood oxygen level, total blood count, (e) the detection and quantification of specific DNA or RNA from biosamples, e.g., cells, viruses, bodily fluids, (f) the sequencing and comparing of genetic sequences in DNA in the chromosomes and mitochondria for genome analysis or (g) to detect reaction products, e.g., during synthesis or purification of pharmaceuticals.

In some embodiments, the analyte can be a biomarker, an environmental marker, or a foodstuff marker. The sample in some instances is a liquid sample, and can be a diagnostic sample (such as saliva, serum, blood, sputum, urine, sweat, lacrima, semen, or mucus); an environmental sample obtained from a river, ocean, lake, rain, snow, sewage, sewage processing runoff, agricultural runoff, industrial runoff, tap water or drinking water; or a foodstuff sample obtained from tap water, drinking water, prepared food, processed food or raw food.

In any embodiment, the sample can be a diagnostic sample obtained from a subject, the analyte can be a biomarker, and the measured the amount of the analyte in the sample can be diagnostic of a disease or a condition.

In any embodiment, the devices, apparatus, systems, and methods in the present invention can further include diagnosing the subject based on information including the measured amount of the biomarker in the sample. In some cases, the diagnosing step includes sending data containing the measured amount of the biomarker to a remote location and receiving a diagnosis based on information including the measurement from the remote location.

In any embodiment, the biomarker can be selected from Tables B1, 2, 3 or 7 as disclosed in U.S. Provisional Application Nos. 62/234,538, 62/293,188, and/or 62/305,123, and/or PCT Application No. PCT/US2016/054025, which are all incorporated in their entireties for all purposes. In some instances, the biomarker is a protein selected from Tables B1, 2, or 3. In some instances, the biomarker is a nucleic acid selected from Tables B2, 3 or 7. In some instances, the biomarker is an infectious agent-derived biomarker selected from Table B2. In some instances, the biomarker is a microRNA (miRNA) selected from Table B7.

In any embodiment, the applying step b) can include isolating miRNA from the sample to generate an isolated miRNA sample, and applying the isolated miRNA sample to the disk-coupled dots-on-pillar antenna (QMAX device) array.

In any embodiment, the QMAX device can contain a plurality of capture agents that each bind to a biomarker selected from Tables B1, B2, B3 and/or B7, wherein the reading step d) includes obtaining a measure of the amount of the plurality of biomarkers in the sample, and wherein the amount of the plurality of biomarkers in the sample is diagnostic of a disease or condition.

In any embodiment, the capture agent can be an antibody epitope and the biomarker can be an antibody that binds to the antibody epitope. In some embodiments, the antibody epitope includes a biomolecule, or a fragment thereof, selected from Tables B4, B5 or B6. In some embodiments, the antibody epitope includes an allergen, or a fragment thereof, selected from Table B5. In some embodiments, the antibody epitope includes an infectious agent-derived biomolecule, or a fragment thereof, selected from Table B6.

In any embodiment, the QMAX device can contain a plurality of antibody epitopes selected from Tables B4, B5 and/or B6, wherein the reading step d) includes obtaining a measure of the amount of a plurality of epitope-binding antibodies in the sample, and wherein the amount of the plurality of epitope-binding antibodies in the sample is diagnostic of a disease or condition.

In any embodiment, the sample can be an environmental sample, and wherein the analyte can be an environmental marker. In some embodiments, the environmental marker is selected from Table B8 in U.S. Provisional Application No. 62/234,538 and/or PCT Application No. PCT/US2016/054025.

In any embodiment, the method can include receiving or providing a report that indicates the safety or harmfulness for a subject to be exposed to the environment from which the sample was obtained.

In any embodiment, the method can include sending data containing the measured amount of the environmental marker to a remote location and receiving a report that indicates the safety or harmfulness for a subject to be exposed to the environment from which the sample was obtained.

In any embodiment, the QMAX device array can include a plurality of capture agents that each binds to an environmental marker selected from Table B8, and wherein the reading step d) can include obtaining a measure of the amount of the plurality of environmental markers in the sample.

In any embodiment, the sample can be a foodstuff sample, wherein the analyte can be a foodstuff marker, and wherein the amount of the foodstuff marker in the sample can correlate with safety of the foodstuff for consumption. In some embodiments, the foodstuff marker is selected from Table B9.

In any embodiment, the method can include receiving or providing a report that indicates the safety or harmfulness for a subject to consume the foodstuff from which the sample is obtained.

In any embodiment, the method can include sending data containing the measured amount of the foodstuff marker to a remote location and receiving a report that indicates the safety or harmfulness for a subject to consume the foodstuff from which the sample is obtained.

In any embodiment, the devices, apparatus, systems, and methods herein disclosed can include a plurality of capture agents that each binds to a foodstuff marker selected from Table B9 from in U.S. Provisional Application No. 62/234,538 and PCT Application No. PCT/US2016/054025, wherein the obtaining can include obtaining a measure of the amount of the plurality of foodstuff markers in the sample, and wherein the amount of the plurality of foodstuff marker in the sample can correlate with safety of the foodstuff for consumption.

Also provided herein are kits that find use in practicing the devices, systems and methods in the present invention.

The amount of sample can be about a drop of a sample. The amount of sample can be the amount collected from a pricked finger or fingerstick. The amount of sample can be the amount collected from a microneedle or a venous draw.

A sample can be used without further processing after obtaining it from the source, or can be processed, e.g., to enrich for an analyte of interest, remove large particulate matter, dissolve or resuspend a solid sample, etc.

Any suitable method of applying a sample to the QMAX device can be employed. Suitable methods can include using a pipet, dropper, syringe, etc. In certain embodiments, when the QMAX device is located on a support in a dipstick format, as described below, the sample can be applied to the QMAX device by dipping a sample-receiving area of the dipstick into the sample.

A sample can be collected at one time, or at a plurality of times. Samples collected over time can be aggregated and/or processed (by applying to a QMAX device and obtaining a measurement of the amount of analyte in the sample, as described herein) individually. In some instances, measurements obtained over time can be aggregated and can be useful for longitudinal analysis over time to facilitate screening, diagnosis, treatment, and/or disease prevention.

Washing the QMAX device to remove unbound sample components can be done in any convenient manner, as described above. In certain embodiments, the surface of the QMAX device is washed using binding buffer to remove unbound sample components.

Detectable labeling of the analyte can be done by any convenient method. The analyte can be labeled directly or indirectly. In direct labeling, the analyte in the sample is labeled before the sample is applied to the QMAX device. In indirect labeling, an unlabeled analyte in a sample is labeled after the sample is applied to the QMAX device to capture the unlabeled analyte, as described below.

### (10) Applications

The devices/apparatus, systems, and methods herein disclosed can be used for various applications (fields and samples). The applications are herein disclosed, listed, described, and/or summarized in PCT Application (designating U.S.) Nos. PCT/US2016/046437 and PCT/US2016/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

In some embodiments, the devices, apparatus, systems, and methods herein disclosed are used in a variety of different application in various field, wherein determination of the presence or absence, quantification, and/or amplification of one or more analytes in a sample are desired. For example, in certain embodiments the subject devices, apparatus, systems, and methods are used in the detection of proteins, peptides, nucleic acids, synthetic compounds, inorganic compounds, organic compounds, bacteria, virus, cells, tissues, nanoparticles, and other molecules, compounds, mixtures and substances thereof. The various fields in which the subject devices, apparatus, systems, and methods can be used include, but are not limited to: diagnostics, management, and/or prevention of human diseases and conditions, diagnostics, management, and/or prevention of veterinary diseases and conditions, diagnostics, management, and/or prevention of plant diseases and conditions, agricultural uses, veterinary uses, food testing, environments testing and decontamination, drug testing and prevention, and others.

The applications of the present invention include, but are not limited to: (a) the detection, purification, quantification, and/or amplification of chemical compounds or biomolecules that correlates with certain diseases, or certain stages of the diseases, e.g., infectious and parasitic disease, injuries, cardiovascular disease, cancer, mental disorders, neuropsychiatric disorders and organic diseases, e.g., pulmonary diseases, renal diseases, (b) the detection, purification, quantification, and/or amplification of cells and/or microorganism, e.g., virus, fungus and bacteria from the environment, e.g., water, soil, or biological samples, e.g., tissues, bodily fluids, (c) the detection, quantification of chemical compounds or biological samples that pose hazard to food safety, human health, or national security, e.g. toxic waste, anthrax, (d) the detection and quantification of vital parameters in medical or physiological monitor, e.g., glucose, blood oxygen level, total blood count, (e) the detection and quantification of specific DNA or RNA from biological samples, e.g., cells, viruses, bodily fluids, (f) the sequencing and comparing of genetic sequences in DNA in the chromosomes and mitochondria for genome analysis or (g) the detection and quantification of reaction products, e.g., during synthesis or purification of pharmaceuticals.

In some embodiments, the subject devices, apparatus, systems, and methods are used in the detection of nucleic acids, proteins, or other molecules or compounds in a sample. In certain embodiments, the devices, apparatus, systems, and methods are used in the rapid, clinical detection and/or quantification of one or more, two or more, or three or more disease biomarkers in a biological sample, e.g., as being employed in the diagnosis, prevention, and/or management of a disease condition in a subject. In certain embodiments, the devices, apparatus, systems, and methods are used in the detection and/or quantification of one or more, two or more, or three or more environmental markers in an environmental sample, e.g. sample obtained from a river, ocean, lake, rain, snow, sewage, sewage processing runoff, agricultural runoff, industrial runoff, tap water or drinking water. In certain embodiments, the devices, apparatus, systems, and methods are used in the detection and/or quantification of one or more, two or more, or three or more foodstuff marks from a food sample obtained from tap water, drinking water, prepared food, processed food or raw food.

In some embodiments, the subject device is part of a microfluidic device. In some embodiments, the subject devices, apparatus, systems, and methods are used to detect a fluorescence or luminescence signal. In some embodiments, the subject devices, apparatus, systems, and methods include, or are used together with, a communication device, such as but not limited to: mobile phones, tablet computers and laptop computers. In some embodiments, the subject devices, apparatus, systems, and methods include, or are used together with, an identifier, such as but not limited to an optical barcode, a radio frequency ID tag, or combinations thereof.

In some embodiments, the sample is a diagnostic sample obtained from a subject, the analyte is a biomarker, and the measured amount of the analyte in the sample is diagnostic of a disease or a condition. In some embodiments, the subject devices, systems and methods further include receiving or providing to the subject a report that indicates the measured amount of the biomarker and a range of measured values for the biomarker in an individual free of or at low risk of having the disease or condition, wherein the measured amount of the biomarker relative to the range of measured values is diagnostic of a disease or condition.

In some embodiments, the sample is an environmental sample, and wherein the analyte is an environmental marker. In some embodiments, the subject devices, systems and methods includes receiving or providing a report that indicates the safety or harmfulness for a subject to be exposed to the environment from which the sample was obtained. In some embodiments, the subject devices, systems and methods include sending data containing the measured amount of the environmental marker to a remote location and receiving a report that indicates the safety or harmfulness for a subject to be exposed to the environment from which the sample was obtained.

In some embodiments, the sample is a foodstuff sample, wherein the analyte is a foodstuff marker, and wherein the amount of the foodstuff marker in the sample correlate with safety of the foodstuff for consumption. In some embodiments, the subject devices, systems and methods include receiving or providing a report that indicates the safety or harmfulness for a subject to consume the foodstuff from which the sample is obtained. In some embodiments, the subject devices, systems and methods include sending data containing the measured amount of the foodstuff marker to a remote location and receiving a report that indicates the safety or harmfulness for a subject to consume the foodstuff from which the sample is obtained.

### (11) Dimensions

The devices, apparatus, systems, and methods herein disclosed can include or use a QMAX device, which can comprise plates and spacers. In some embodiments, the dimension of the individual components of the QMAX device and its adaptor are listed, described and/or summarized in PCT Application (designating U.S.) No. PCT/US2016/046437 filed on Aug. 10, 2016, and U.S. Provisional Application Nos. 62,431,639 filed on Dec. 9, 2016 and 62/456,287 filed on Feb. 8, 2017, which are all hereby incorporated by reference by their entireties.

In some embodiments, the dimensions are listed in the Tables below:

Plates:

| Para-meters | Embodiments | Preferred Embodiments |
|---|---|---|
| Shape | round, ellipse, rectangle, triangle, polygonal, ring- | at least one of the two (or |
| | shaped, or any superposition of these shapes; the | more) plates of the QMAX |
| | two (or more) plates of the QMAX card can have | card has round corners for |
| | the same size and/or shape, or different size and/ | user safety concerns, wherein |
| | or shape; | the round corners have a |
| | | diameter of 100 μm or less, 200 |
| | | μm or less, 500 μm or less, |
| | | 1 mm or less, 2 mm or less, 5 |
| | | mm or less, 10 mm or less, 50 mm |
| | | or less, or in a range between |
| | | any two of the values. |
| Thickness | the average thickness for at least one of the plates | For at least one of the |
| | is 2 nm or less, 10 nm or less, 100 nm or less, 200 | plates is in the range of 0.5 |
| | nm or less, 500 nm or less, 1000 nm or less, 2 μm | to 1.5 mm; around 1 mm; in |
| | (micron) or less, 5 μm or less, 10 μm or less, 20 | the range of 0.15 to 0.2 mm; |
| | μm or less, 50 μm or less, 100 μm or less, 150 μm | or around 0.175 mm |
| | or less, 200 μm or less, 300 μm or less, 500 μm or | |
| | less, 800 μm or less, 1 mm (millimeter) or less, 2 | |
| | mm or less, 3 mm or less, 5 mm or less, 10 mm or | |
| | less, 20 mm or less, 50 mm or less, 100 mm or less, | |
| | 500 mm or less, or in a range between any two of | |
| | these values | |
| Lateral Area | For at least one of the plate is 1 mm2 (square | For at least one plate of the |
| | millimeter) or less, 10 mm2 or less, 25 mm2 or | QMAX card is in the range of |
| | less, 50 mm2 or less, 75 mm2 or less, 1 cm2 | 500 to 1000 mm²; or around |
| | (square centimeter) or less, 2 cm2 or less, 3 cm2 | 750 mm². |
| | or less, 4 cm2 or less, 5 cm2 or less, 10 cm2 or | |
| | less, 100 cm2 or less, 500 cm2 or less, 1000 cm2 | |
| | or less, 5000 cm2 or less, 10,000 cm2 or less, | |
| | 10,000 cm2 or less, or in a range between any two | |
| | of these values | |
| Lateral Linear | For at least one of the plates of the QMAX card is | For at least one plate of the |
| Dimension (width, | 1 mm or less, 5 mm or less, 10 mm or less, 15 mm | QMAX card is in the range of |
| length, or | or less, 20 mm or less, 25 mm or less, 30 mm or | 20 to 30 mm; or around 24 mm |
| diameter, etc.) | less, 35 mm or less, 40 mm or less, 45 mm or less, | |
| | 50 mm or less, 100 mm or less, 200 mm or less, | |
| | 500 mm or less, 1000 mm or less, 5000 mm or less, | |
| | or in a range between any two of these values | |
| Recess width | 1 μm or less, 10 μm or less, 20 μm or less, 30 μm | In the range of 1 mm to 10 |
| | or less, 40 μm or less, 50 μm or less, 100 μm or | mm; Or About 5 mm |
| | less, 200 μm or less, 300 μm or less, 400 μm or | |
| | less, 500 μm or less, 7500 μm or less, 1 mm or | |
| | less, 5 mm or less, 10 mm or less, 100 mm or less, | |
| | or 1000 mm or less, or in a range between any two | |
| | of these values. | |

Hinge:

| Parame- | | Preferred |
|---|---|---|
| ters | Embodiments | Embodiments |
| Number | 1, 2, 3, 4, 5, or more | 1 or 2 |
| Length of | 1 mm or less, 2 mm or less, 3 mm or | In the range of |
| Hinge Joint | less, 4 mm or less, 5 mm or less, | 5 mm to 30 mm. |
| | 10 mm or less, 15 mm or less, 20 mm | |
| | or less, 25 mm or less, 30 mm or | |
| | less, 40 mm or less, 50 mm or less, | |
| | 100 mm or less, 200 mm or less, or | |
| | 500 mm or less, or in a range | |
| | between any two of these values | |
| Ratio (hinge | 1.5 or less, 1 or less, 0.9 or less, | In the range of |
| joint length | 0.8 or less, 0.7 or less, 0.6 or | 0.2 to 1; or |
| vs. aligning | less, 0.5 or less, 0.4 or less, 0.3 | about 1 |
| plate edge | or less, 0.2 or less, 0.1 or less, | |
| length | 0.05 or less or in a range between | |
| | any two of these values. | |
| Area | 1 mm² or less, 5 mm² or less, 10 | In the range of |
| | mm² or less, 20 mm² or less, 30 mm² | 20 to 200 mm²; or |
| | or less, 40 mm² or less, 50 mm² or | about 120 mm² |
| | less, 100 mm² or less, 200 mm² or | |
| | less, 500 mm² or less, or in a | |
| | range between any of the two values | |
| Ratio (hinge | 1 or less, 0.9 or less, 0.8 or less, | In the range of |
| area vs. | 0.7 or less, 0.6 or less, 0.5 or | 0.05 to 0.2, |
| plate area) | less, 0.4 or less, 0.3 or less, 0.2 | around 0.15 |
| | or less, 0.1 or less, 0.05 or less, | |
| | 0.01 or less or in a range between | |
| | any two of these values | |
| Max. Open | 15 or less, 30 or less, 45 or less, | In the range of |
| Degree | 60 or less, 75 or less, 90 or less, | 90 to 180 degrees |
| | 105 or less, 120 or less, 135 or | |
| | less, 150 or less, 165 or less, 180 | |
| | or less, 195 or less, 210 or less, | |
| | 225 or less, 240 or less, 255 or | |
| | less, 270 or less, 285 or less, 300 | |
| | or less, 315 or less, 330 or less, | |
| | 345 or less or 360 or less degrees, | |
| | or in a range between any two of | |
| | these values | |
| No. of | 1, 2, 3, 4, 5, or more | 1 or 2 |
| Layers | | |
| Layer | 0.1 μm or less, 1 μm or less, 2 μm | In the range of |
| thickness | or less, 3 μm or less, 5 μm or less, | 20 μm to 1 mm; |
| | 10 μm or less, 20 μm or less, 30 μm | or Around 50 μm |
| | or less, 50 μm or less, 100 μm or | |
| | less, 200 μm or less, 300 μm or less, | |
| | 500 μm or less, 1 mm or less, 2 mm | |
| | or less, and a range between any | |
| | two of these values | |
| Angle- | Limiting the angle adjustment with | No more than ±2 |
| maintain- | no more than ±90, ±45, ±30, ±25, | |
| ing | ±20, ±15, ±10, ±8, ±6, ±5, ±4, ±3, | |
| | ±2, or ±1, or in a range between | |
| | any two of these values | |

Notch:

| | | Preferred |
|---|---|---|
| Parameters | Embodiments | Embodiments |
| Number | 1, 2, 3, 4, 5, or more | 1 or 2 |
| Shape | round, ellipse, rectangle, triangle, | Part of a |
| | polygon, ring-shaped, or any | circle |
| | superposition or portion of these | |
| | shapes. | |
| Positioning | Any location along any edge except | |
| | the hinge edge, or any corner joint | |
| | by non-hinge edges | |
| Lateral | 1 mm or less, 2.5 mm or less, 5 mm | In the range of 5 |
| Linear | or less, 10 mm or less, 15 mm or | mm to 15 mm; or |
| Dimension | less, 20 mm or less, 25 mm or less, | about 10 mm |
| (Length | 30 mm or less, 40 mm or less, 50 mm | |
| along the | or less, or in a range between any | |
| edge, radius, | two of these values | |
| etc.) | | |
| Area | 1 mm² (square millimeter) or less, | In the range of 10 |
| | 10 mm² or less, 25 mm² or less, | to 150 mm²; or |
| | 50 mm² or less, 75 mm² or less | about 50 mm² |
| | or in a range between any two of these | |
| | values. | |

Trench:

| | | Preferred |
|---|---|---|
| Parameters | Embodiments | Embodiments |
| Number | 1, 2, 3, 4, 5, or more | 1 or 2 |
| Shape | Closed (round, ellipse, rectangle, | |
| | triangle, polygon, ring-shaped, or | |
| | any superposition or portion of | |
| | these shapes) or open-ended | |
| | (straight line, curved line, arc, | |
| | branched tree, or any other shape | |
| | with open endings); | |
| Length | 0.001 mm or less, 0.005 mm or less, | |
| | 0.01 mm or less, 0.05 mm or less, | |
| | 0.1 mm or less, 0.5 mm or less, 1 | |
| | mm or less, 2 mm or less, 5 mm or | |
| | less, 10 mm or less, 20 mm or less, | |
| | 50 mm or less, 100 mm or less, or | |
| | in a range between any two of these | |
| | values | |
| Cross- | 0.001 mm² or less, 0.005 mm² or less, | |
| sectional | 0.01 mm² or less, 0.05 mm² or less, | |
| Area | 0.1 mm² or less, 0.5 mm² or less, 1 | |
| | mm² or less, 2 mm² or less, 5 mm² or | |
| | less, 10 mm² or less, 20 mm² or less, | |
| | or in a range between any two of | |
| | these values. | |
| Volume | 0.1 uL or more, 0.5 uL or more, 1 uL | In the range of 1 |
| | or more, 2 uL or more, 5 uL or more, | uL to 20 uL; or |
| | 10 uL or more, 30 uL or more, 50 uL | About 5 uL |
| | or more, 100 uL or more, 500 uL or | |
| | more, 1 mL or more, or in a range | |
| | between any two of these values | |

Receptacle Slot

| | | Preferred |
|---|---|---|
| Parameters | Embodiments | Embodiments |
| Shape of | round, ellipse, rectangle, triangle, | |
| receiving area | polygon, ring-shaped, or any | |
| | superposition of these shapes; | |
| Difference | 100 nm, 500 nm, 1 μm, 2 μm, 5 μm, 10 | In the range of |
| between sliding | μm, 50 μm, 100 μm, 300 μm, 500 μm, 1 | 50 to 300 μm; or |
| track gap size | mm, 2 mm, 5 mm, 1 cm, or in a range | about 75 μm |
| and card thickness | between any two of the values. | |
| Difference | 1 mm² (square millimeter) or less, 10 | |
| between | mm² or less, 25 mm² or less, 50 mm² or | |
| receiving | less, 75 mm² or less, 1 cm² (square | |
| area and card | centimeter) or less, 2 cm² or less, 3 | |
| area | cm² or less, 4 cm² or less, 5 cm² or | |
| | less, 10 cm² or less, 100 cm² or less, | |
| | or in a range between any of the two | |
| | values. | |

### (12) Cloud

The devices/apparatus, systems, and methods herein disclosed can employ cloud technology for data transfer, storage, and/or analysis. The related cloud technologies are herein disclosed, listed, described, and/or summarized in PCT Application (designating U.S.) Nos. PCT/US2016/046437 and PCT/US2016/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

In some embodiments, the cloud storage and computing technologies can involve a cloud database. Merely by way of example, the cloud platform can include a private cloud, a public cloud, a hybrid cloud, a community cloud, a distributed cloud, an inter-cloud, a multi-cloud, or the like, or any combination thereof. In some embodiments, the mobile device (e.g. smartphone) can be connected to the cloud through any type of network, including a local area network (LAN) or a wide area network (WAN).

In some embodiments, the data (e.g. images of the sample) related to the sample is sent to the cloud without processing by the mobile device and further analysis can be conducted remotely. In some embodiments, the data related to the sample is processed by the mobile device and the results are sent to the cloud. In some embodiments, both the raw data and the results are transmitted to the cloud.

### ADDITIONAL NOTES

Further examples of inventive subject matter according to the present disclosure are described in the following enumerated paragraphs.

It must be noted that as used herein and in the appended claims, the singular forms “a”, “an”, and “the” include plural referents unless the context clearly dictates otherwise, e.g., when the word “single” is used. For example, reference to “an analyte” includes a single analyte and multiple analytes, reference to “a capture agent” includes a single capture agent and multiple capture agents, reference to “a detection agent” includes a single detection agent and multiple detection agents, and reference to “an agent” includes a single agent and multiple agents.

As used herein, the terms “adapted” and “configured” mean that the element, component, or other subject matter is designed and/or intended to perform a given function. Thus, the use of the terms “adapted” and “configured” should not be construed to mean that a given element, component, or other subject matter is simply “capable of” performing a given function. Similarly, subject matter that is recited as being configured to perform a particular function may additionally or alternatively be described as being operative to perform that function.

As used herein, the phrase, “for example,” the phrase, “as an example,” and/or simply the terms “example” and “exemplary” when used with reference to one or more components, features, details, structures, embodiments, and/or methods according to the present disclosure, are intended to convey that the described component, feature, detail, structure, embodiment, and/or method is an illustrative, non-exclusive example of components, features, details, structures, embodiments, and/or methods according to the present disclosure. Thus, the described component, feature, detail, structure, embodiment, and/or method is not intended to be limiting, required, or exclusive/exhaustive; and other components, features, details, structures, embodiments, and/or methods, including structurally and/or functionally similar and/or equivalent components, features, details, structures, embodiments, and/or methods, are also within the scope of the present disclosure.

As used herein, the phrases “at least one of” and “one or more of,” in reference to a list of more than one entity, means any one or more of the entity in the list of entity, and is not limited to at least one of each and every entity specifically listed within the list of entity. For example, “at least one of A and B” (or, equivalently, “at least one of A or B,” or, equivalently, “at least one of A and/or B”) may refer to A alone, B alone, or the combination of A and B.

As used herein, the term “and/or” placed between a first entity and a second entity means one of (1) the first entity, (2) the second entity, and (3) the first entity and the second entity. Multiple entity listed with “and/or” should be construed in the same manner, i.e., “one or more” of the entity so conjoined. Other entity may optionally be present other than the entity specifically identified by the “and/or” clause, whether related or unrelated to those entities specifically identified.

Where numerical ranges are mentioned herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, unless otherwise indicated or otherwise evident from the context and understanding of one of ordinary skill in the art.

In the event that any patents, patent applications, or other references are incorporated by reference herein and (1) define a term in a manner that is inconsistent with and/or (2) are otherwise inconsistent with, either the non-incorporated portion of the present disclosure or any of the other incorporated references, the non-incorporated portion of the present disclosure shall control, and the term or incorporated disclosure therein shall only control with respect to the reference in which the term is defined and/or the incorporated disclosure was present originally.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

One with skill in the art will appreciate that the present invention is not limited in its application to the details of construction, the arrangements of components, category selections, weightings, pre-determined signal limits, or the steps set forth in the description or drawings herein. The invention is capable of other embodiments and of being practiced or being carried out in many different ways.

What is claimed is:

1. A device for illuminating and imaging an object, comprising:
(a) an imager having a lens; and
(b) a passive illuminator; and
(c) an adaptor housing that has an exit aperture for positioning an imager
wherein the passive illuminator is on the adaptor; and
wherein the adaptor housing is configured to reduce ambient light outside the adaptor housing entering inside adaptor housing.

2. The device of claim 1, wherein the adaptor housing further comprises a slot for inserting a sample holder into the adaptor housing and the passive illuminator is position around and outside peripheral of the exit aperture.

3. An apparatus for illuminating and imaging an object, comprising:
(a) a mobile phone that has a camera and a light source; and
(b) the device of claim 1.

4. A method for illuminating and imaging an object, the method comprising the steps of:
(a) providing the device of claim 1;
(b) providing an adaptor housing; and
(c) providing a mobile phone that has a camera and a light source,
wherein the adaptor housing has an exit aperture for positioning the imager,
wherein the adaptor housing is configured to reduce ambient light outside the adaptor housing entering the adaptor housing, and
wherein the adaptor housing is configured to attach to the mobile phone.

5. A method for illuminating and imaging a liquid sample between two parallel plates in an assay device, the method comprising the steps of:
(a) impinging light into one or two light-guides each connecting to one end of the passive illuminator;
(b) causing the impinging light to travel through each light-guide to reach the corresponding end of the passive illuminator;
(c) causing light to be emitted from a side wall of the passive illuminator after the impinging light enters the corresponding end of the passive illuminator;
(d) generating illumination light from the light emitted from the side wall of the passive illuminator;
(e) illuminating the liquid sample through one of the parallel plates with the illumination light; and
(f) imaging the liquid sample with an imaging sensor through a lens.

6. An apparatus for using with a smartphone to read an assay device having two parallel plates, the smartphone having a camera and a light source, the apparatus comprising:
(a) one or two light-guides each having an end thereof aligned with the entrance aperture of an optics chamber to cause light entering such end of the light-guide to travel through the light-guide to reach a corresponding end of the passive illuminator; and
(b) a passive illuminator for illuminating a liquid sample between the two parallel plates in the assay device by generating diffusive light sideways from areas surrounding an optical axis of a lens in the camera of the smartphone,
wherein the passive illuminator has a first end optically coupled to a second end of the light-guide to cause light received at the first end of each light-guide to travel through the light-guide to enter the first end of the passive illuminator.

7. The apparatus of claim 6, further comprising a diffuser for generating diffusive light sideways from areas surrounding the optical axis of the lens in the camera of the smartphone to illuminate the liquid sample between the two parallel plates in the assay device.

8. The device of claim 1, wherein the passive illuminator is in the form of a ring configured to surround an optical axis of a lens in the camera of the smartphone when the apparatus is engaged with the smartphone.

9. The device of claim 1, further comprising an auxiliary lens having an optical axis thereof aligned with the optical axis of the lens in the camera of the smartphone when the apparatus is engaged with the smartphone, wherein the auxiliary lens has a diameter that is at least 2 mm, 3 mm, 4 mm, 5 mm, 10 mm, 15 mm, 20 mm, 25 mm, 30 mm, 40 mm, or 50 mm, or in a range between any of the two values.

10. The device of claim 1, further comprising an optical condenser configured to be placed in front of the light source of the smartphone when the apparatus is engaged with the smartphone, or an optical condenser aligned with the entrance aperture of an optics chamber.

11. The apparatus of claim 7, wherein the diffuser comprises at least one of the following:
(a) polished surfaces on both sides;
(b) a volume diffusive material which can be but not limited to opaque white glass and opaque white plastic, wherein the transmissivity of the volume diffusive material is at least 40%, 60%, 80%, 90% or in a range between any of the two values; and
(c) at least one textured surface,
wherein the volume diffusive material can be but not limited to opaque white glass and opaque white plastic,
wherein the transmissivity of the volume diffusive material is at least 40%, 60%, 80%, 90% or in a range between any of the two values, and
wherein the grit of the textured surface is at least 100, 200, 400, 600, 800, 1,000, 2,000, or in a range between any of the two values.

12. The apparatus of claim 7, further comprising a reflector configured to reflect light emitted from the passive illuminator towards the diffuser, or a reflector configured to reflect light emitted from the passive illuminator towards the exposure aperture of the optics chamber.

13. The device of claim 6, further comprising:
(a) a receptacle slot operative to hold the assay device while exposing at least part of a first one of the two parallel plate in the assay device to a lens in the camera of the smartphone when the assay device is inserted into the receptacle slot and the apparatus is engaged with the smartphone; or
(b) a receptacle slot operative to hold the assay device while exposing at least part of a first one of the two parallel plate in the assay device to the exposure aperture of the optics chamber when the assay device is inserted into the receptacle slot; or
(c) a receptacle slot having two side walls forming a cavity for holding the assay device therein, wherein one of the two side walls has an opening for forming the exposure aperture of the optics chamber,
wherein the light-guide has the first end configured to receive light from the light source of the smartphone when the apparatus is engaged with the smartphone.

14. The device of claim 1, further comprising:
(a) an optics chamber having an entrance aperture;
(b) an exit aperture at a first side of the optics chamber; and
(c) an exposure aperture at a second side of the optics chamber,
wherein the light-guide has the first end aligned with the entrance aperture of the optics chamber,
wherein each of the entrance aperture, the exit aperture, and the exposure aperture is covered with a window.

15. The device of claim 14, wherein the exit aperture at the first side of the optics chamber is aligned with the exposure aperture at the second side of the optics chamber for exposing optically at least part of the first one of the two parallel plate in the assay device to the exit aperture of the optics chamber through the exposure aperture of the optics chamber when the assay device is inserted into the receptacle slot.

16. The device of claim 15, further comprising an auxiliary lens aligned with the exit aperture of the optics chamber, or an auxiliary lens located between the passive illuminator and the receptacle slot operative to hold the assay device, or an auxiliary having an optical axis thereof coaxially aligned with an optical axis of the lens in the camera of the smartphone when the apparatus is engaged with the smartphone.

17. The device of claim 1, further comprising:
(a) a diffuser placed at a predetermined distance from the passive illuminator; and
(b) an opening on the diffuser configured to expose to the camera of the smartphone at least a part of the exposure aperture in the optics chamber when the apparatus is engaged with the smartphone,
wherein the diffuser is configured to intercept all light path directly between the passive illuminator and the exposure aperture of the optics chamber.

18. The device of claim 2, wherein the distance between the passive illuminator and the outside peripheral of the imager is in a range of 2 mm to 50 mm.

19. The device of claim 1, wherein the passive illuminator is formed by a side illumination fiber, wherein the side illumination fiber comprises a core and a cladding layer, and wherein the ratio of transmissivity to reflectivity at the interface between the core and cladding layer is at least 1:100, 1:10, 1:1, or in a range between any of the two values, or wherein the passive illuminator is formed by a side illumination fiber, and wherein the side illumination fiber is made of but not limited to flexible polymers, plastic, glass and rigid dielectric materials.

20. The device of claim 1, wherein the passive illuminator is rotationally symmetric or rotationally non-symmetric, or the passive illuminator is in the form of a circle having a diameter thereof in a range between 5 mm and 100 mm, or the passive illuminator is in the form of a convex polygon, a star polygon, an ellipse, or a circle, or the passive illuminator is formed by a single piece of side illumination fiber or by at least two segments of side illumination fibers, or the passive illuminator has a substantially uniform cross-section.
